(19) United States
(12) Patent Application Publication
(10) Pub. No.: US 2017/0014089 A1
(43) Pub. Date: Jan. 19, 2017

(54) ALERT INFORMATION PRESENTING APPARATUS, ALERT INFORMATION PRESENTING METHOD, AND BLOOD PRESSURE INFORMATION OUTPUTTING METHOD

(71) Applicant: Panasonic Intellectual Property Management Co., Ltd., Osaka (JP)

(72) Inventors: KENTA MURAKAMI, Nara (JP); MOTOTAKA YOSHIOKA, Osaka (JP); JUN OZAWA, Nara (JP)

(21) Appl. No.: 15/190,355

(22) Filed: Jun. 23, 2016

(30) Foreign Application Priority Data
Jul. 17, 2015 (JP) 2015-143388

(51) Int. Cl. A61B 5/00; A61B 5/11; A61B 5/0205

(52) U.S. Cl. CPC A61B 5/746; A61B 5/0205; A61B 5/1123; A61B 5/0077; A61B 5/486; A61B 5/7275; A61B 5/02125

(57) ABSTRACT

An alert information presenting apparatus includes an undressing condition determiner that determines, by using a movement speed of the user, a second time period indicating a time period before undressing, and a third time period indicating a time period after undressing, a heartbeat acquirer that acquires a heartbeat timing in the second time period and a heartbeat timing in the third time period, a pulse wave acquirer that acquires a pulse wave timing in the second time period and a pulse wave timing in the third time period, a blood pressure calculator that calculates a pre-undressing blood pressure by using a pre-undressing heartbeat timing and a pre-undressing pulse wave timing, and calculates a post-undressing blood pressure by using a post-undressing heartbeat timing and a post-undressing pulse wave timing, and an information presenter that presents the user with alert information based on a difference between the pre-undressing and post-undressing blood pressures.

### BACKGROUND

1. Technical Field

The present disclosure relates to an apparatus and method for presenting alert information about bathing or other activities, and a blood pressure information outputting method.

2. Description of the Related Art

Japanese Unexamined Patent Application Publication No. 2012-125281 discloses an apparatus that extracts, while the user is bathing, pulse wave from the fingertip, and heartbeat from a pillow part placed under the back of the head, estimates blood pressure based on the time difference between the extracted pulse wave and the extracted heartbeat, and causes water to be drained in case of abnormal blood pressure variation.

However, the technique disclosed in Japanese Unexamined Patent Application Publication No. 2012-125281 does not make it possible to prevent the user from putting himself or herself at risk for potentially dangerous conditions.

### SUMMARY

One non-limiting and exemplary embodiment provides an alert information presenting apparatus capable of keeping the user away from potentially dangerous conditions.

In one general aspect, the techniques disclosed here feature an alert information presenting apparatus including a signal acquirer that acquires a receive signal including a radio wave reflected by a user, an undressing condition determiner that determines, by using a movement speed of the user determined by using the receive signal and indicating a speed of a movement performed by the user, a first time period indicating a period of time during which the user is undressing, a second time period immediately preceding the first time period and indicating a period of time before the user undresses, and a third time period immediately following the first time period and indicating a period of time after the user undresses, a heartbeat acquirer that acquires, by using the receive signal, a pre-undressing heartbeat timing and a post-undressing heartbeat timing, the pre-undressing heartbeat timing indicating a heartbeat timing in the second time period, the post-undressing heartbeat timing indicating a heartbeat timing in the third time period, a pulse wave acquirer that acquires a pre-undressing pulse wave timing and a post-undressing pulse wave timing, the pre-undressing pulse wave timing indicating a pulse wave timing in the second time period, the post-undressing pulse wave timing indicating a pulse wave timing in the third time period, a blood pressure calculator that calculates a pre-undressing blood pressure of the user and a post-undressing blood pressure of the user, the pre-undressing blood pressure being calculated by using a time difference between the pre-undressing heartbeat timing and the pre-undressing pulse wave timing, the post-undressing blood pressure being calculated by using a time difference between the post-undressing heartbeat timing and the post-undressing pulse wave timing, and an information presenter that presents the user with alert information based on a difference between the pre-undressing blood pressure and the post-undressing blood pressure.

The present disclosure makes it possible to keep the user away from potentially dangerous conditions.

It should be noted that general or specific embodiments may be implemented as a system, a method, an integrated circuit, a computer program, a computer readable storage medium, or any selective combination thereof. Non-limiting examples of a computer readable storage medium include a non-volatile recording medium such as a compact disc-read only memory (CD-ROM).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram illustrating the configuration of an alert information presenting apparatus according to Embodiment 1;

FIG. 2A is an external view of an alert information presenting apparatus according to Embodiment 1;

FIG. 2B schematically illustrates how an alert information presenting apparatus is used by the user according to Embodiment 1;

FIG. 3 illustrates the principle for calculation of heartbeat timing according to Embodiment 1;

FIG. 4 illustrates changes in the movement (distance) of the chest obtained from a receive signal according to Embodiment 1;

FIG. 5 illustrates the relationship between blood pressure and time points at which blood pressure is measured;

FIG. 6 illustrates a method according to Embodiment 1 for detecting, by using a receive signal, the presence of the user and the movement speed of the user;

FIG. 7 illustrates an example of user's undressing conditions (undressing movements) determined by using millimeter waves according to Embodiment 1;

FIG. 8 illustrates a method according to Embodiment 1 for obtaining information about the user's body from an azimuth, an elevation angle, and a distance detected by using a signal acquirer;

FIG. 9 illustrates a method according to Embodiment 1 for determining the presence of a body;

FIG. 10 illustrates a method according to Embodiment 1 for determining an undressing movement performed by the user;

FIG. 11 illustrates patterns of movement speeds in various areas used to determine various undressing movements according to Embodiment 1;

FIG. 12 illustrates movement speed thresholds for various areas set for each individual undressing movement;

FIG. 13 illustrates movement speeds thresholds for various areas in movement B and movements C1 and C2 according to Embodiment 1;

FIG. 14 illustrates an example of a database storing thresholds that change in time series according to Embodiment 1;

FIG. 15 illustrates undressing determination and the timing of blood pressure measurements according to Embodiment 1;

FIG. 16 illustrates patterns of user's undressing order according to Embodiment 1;

FIG. 17 illustrates blood pressure measurement performed before undressing according to Embodiment 1;

FIG. 18 illustrates an example of calculation of heartbeat timing according to Embodiment 1;

FIG. 19 illustrates an example of changes in the luminance of a skin image according to Embodiment 1;

FIG. 20 illustrates an example of calculation of pulse wave timing according to Embodiment 1;

FIG. 21 illustrates calculation of blood pressure based on pulse wave timing and heartbeat timing according to Embodiment 1;

FIG. 22 illustrates an example of alert determination (determination of whether to permit bathing) according to Embodiment 1;

FIG. 23 illustrates another example of alert determination (determination of whether to permit bathing) according to Embodiment 1;

FIG. 24 illustrates a specific example of when it is determined to permit bathing, after it is once determined not to permit bathing based on pattern 1 illustrated in FIG. 22;

FIG. 25 illustrates an example of information presented to the user by an information presenter according to Embodiment 1;

FIG. 26 is a flowchart illustrating a procedure for the processing performed by an alert information presenting apparatus according to Embodiment 1;

FIG. 27 is a flowchart illustrating a more specific procedure for the processing performed by an alert information presenting apparatus according to Embodiment 1;

FIG. 28 is a flowchart illustrating a detailed procedure for determining whether to measure blood pressure according to Embodiment 1;

FIG. 29 is a flowchart illustrating a detailed procedure for measuring blood pressure according to Embodiment 1;

FIG. 30 is a flowchart illustrating a detailed procedure for determining whether undressing is completed according to Embodiment 1;

FIG. 31 illustrates an example of storing of a peculiar pattern of undressing order according to Embodiment 1;

FIG. 32 illustrates determination of undressing in sitting position mode according to Embodiment 1;

FIG. 33 illustrates an example of undressing movements performed by the user while in sitting position according to Embodiment 1;

FIG. 34 is a block diagram illustrating the configuration of an alert information presenting apparatus according to Embodiment 2;

FIG. 35 illustrates how pulse wave timing and heartbeat timing are acquired by an alert information presenting apparatus according to Embodiment 2;

FIG. 36 illustrates an exemplary use and configuration of a sensor mat according to Embodiment 2;

FIG. 37 illustrates an example of pulse wave signals obtained by a plurality of pulse wave sensors according to Embodiment 2;

FIG. 38 is a flowchart illustrating a procedure for selecting a pulse wave sensor according to Embodiment 2;

FIG. 39 is a block diagram illustrating the configuration of an alert information presenting apparatus according to Modification 1 of Embodiment 1;

FIG. 40 illustrates how the user's undressing process is determined by an alert information presenting apparatus according to Modification 1 of Embodiment 1;

FIG. 41 is a flowchart illustrating a procedure for determining undressing condition according to Modification 1 of Embodiment 1;

FIG. 42 is a block diagram illustrating the configuration of an alert information presenting apparatus according to Modification 2 of Embodiment 1;

FIG. 43 illustrates an overview of Modification 2 of Embodiment 1;

FIG. 44 is a flowchart illustrating a procedure for determining undressing condition according to Modification 2 of Embodiment 1;

FIG. 45 is a flowchart illustrating a procedure for presenting information according to Modification 2 of Embodiment 1;

FIG. 46 is a block diagram illustrating the configuration of an alert information presenting apparatus according to Embodiment 3;

FIG. 47 illustrates how user's dressing condition after bathing is determined according to Embodiment 3;

FIG. 48 illustrates how, according to Embodiment 3, a signal indicating entry of the user into the changing room immediately after bathing is acquired by a signal acquirer, and the signal is transmitted to a dressing condition determiner;

FIG. 49 illustrates how, according to Embodiment 3, the user is detected by using, for example, a sensor embedded in a drainage mat;

FIG. 50 illustrates an example of patterns of changes in user's blood pressure following the entry of the user into the changing room after bathing according to Embodiment 3;

FIG. 51 illustrates an example of patterns of changes in user's blood pressure following the entry of the user into the changing room after bathing according to Embodiment 3;

FIG. 52 illustrates an example of a pattern of changes in user's blood pressure following the entry of the user into the changing room after bathing according to Embodiment 3;

FIG. 53 is a flowchart illustrating operation of an alert information presenting apparatus according to Embodiment 3; and

FIG. 54 is a flowchart illustrating operation of an alert information presenting apparatus according to Embodiment 3.

### DETAILED DESCRIPTION

### Underlying Knowledge Forming Basis of the Present Disclosure

The present inventor has found the following problems with regard to the technique according to Japanese Unexamined Patent Application Publication No. 2012-125281 described in the “Description of the Related Art” section.

Specifically, although the technique according to Japanese Unexamined Patent Application Publication No. 2012-125281 allows detection of variations in person's blood pressure that occur during bathing, the technique does not allow detection of variations in person's blood pressure that occur before bathing. A common possible cause of accidents such as myocardial infarction or cerebral infarction in bathrooms is variations in blood pressure that begin before bathing, for example, in changing rooms. Blood pressure variation already takes place before bathing. For example, when a person undresses in winter time, the body is exposed to a low air temperature, and the resulting drop in body temperature causes blood pressure to increase. The subsequent rapid rise in body temperature associated with bathing causes an acute drop in blood pressure, which can also lead to accidents such as dizziness or falling over. This suggests that detecting pre-bathing blood pressure variation is important to prevent these accidents. That is, to prevent accidents caused by an elevation in blood pressure and a drop in blood pressure following the elevation in blood pressure, it is important to detect blood pressure elevation and take appropriate measures, such as by providing an alert to that effect.

Accordingly, the present disclosure determines potentially dangerous conditions based on variations in blood pressure during undressing that takes place before bathing, and presents alert information to keep the user away from potentially dangerous conditions.

That is, to address the above-mentioned problem, an aspect of the present disclosure provides an alert information presenting apparatus including a signal acquirer that acquires a receive signal including a radio wave reflected by a user, an undressing condition determiner that determines, by using a movement speed of the user determined by using the receive signal and indicating a speed of a movement performed by the user, a first time period indicating a period of time during which the user is undressing, a second time period immediately preceding the first time period and indicating a period of time before the user undresses, and a third time period immediately following the first time period and indicating a period of time after the user undresses, a heartbeat acquirer that acquires, by using the receive signal, a pre-undressing heartbeat timing and a post-undressing heartbeat timing, the pre-undressing heartbeat timing indicating a heartbeat timing in the second time period, the post-undressing heartbeat timing indicating a heartbeat timing in the third time period, a pulse wave acquirer that acquires a pre-undressing pulse wave timing and a post-undressing pulse wave timing, the pre-undressing pulse wave timing indicating a pulse wave timing in the second time period, the post-undressing pulse wave timing indicating a pulse wave timing in the third time period, a blood pressure calculator that calculates a pre-undressing blood pressure of the user and a post-undressing blood pressure of the user, the pre-undressing blood pressure being calculated by using a time difference between the pre-undressing heartbeat timing and the pre-undressing pulse wave timing, the post-undressing blood pressure being calculated by using a time difference between the post-undressing heartbeat timing and the post-undressing pulse wave timing, and an information presenter that presents the user with alert information based on a difference between the pre-undressing blood pressure and the post-undressing blood pressure. For example, the heartbeat acquirer acquires, as the pre-undressing heartbeat timing, a time point corresponding to a peak of a time waveform in the second time period of the receive signal, and the heartbeat acquirer acquires, as the post-undressing heartbeat timing, a time point corresponding to a peak of a time waveform in the third time period of the receive signal. For example, the alert information presenting apparatus further includes an image acquirer that acquires an image including a skin of the user, the pulse wave acquirer acquires, as the pre-undressing pulse wave timing, a time point corresponding to a peak of a time waveform in the second time period of a signal that indicates time variation of a luminance of the skin of the user in the image, and the pulse wave acquirer acquires, as the post-undressing pulse wave timing, a time point corresponding to a peak of a time waveform in the third time period of the signal that indicates the time variation of the luminance of the skin of the user in the image.

As a result, alert information is presented to the user based on the difference between the pre-undressing blood pressure and the post-undressing blood pressure, that is, the difference in blood pressure before and after the user performs an undressing movement. This makes it possible to present the user with alert information based on blood pressure variation caused by the undressing movement. As a result, appropriate alert information can be presented to the user before, for example, the user takes a bath, thus keeping the user away from potentially dangerous conditions.

That is, according to the present disclosure, user's undressing condition while the user undresses before bathing is determined, and blood pressures in various states of undressing are measured to enable observation of variations in blood pressure during the undressing process (undressing sequence). Then, whether taking a bath is potentially dangerous for the user can be determined based on the amount of blood pressure variation that occurs during the undressing process.

In some embodiments, the undressing condition determiner determines, as the first time period, a period of time during which the receive signal shows a movement speed equal to or greater than a first threshold, the undressing condition determiner determines, as the second time period, a period of time immediately preceding the first time period and during which the receive signal shows a movement speed less than the first threshold, and the undressing condition determiner determines, as the third time period, a period of time immediately following the first time period and during which the receive signal shows a movement speed less than the first threshold. For example, the first threshold is 10 cm/s to 15 cm/s.

As a result, pre-undressing and post-undressing blood pressures are measured when the speed of movement performed by the user is low. This allows blood pressure variation to be properly identified while minimizing errors attributed to undressing movement.

In some embodiments, the information presenter presents, if the difference between the pre-undressing blood pressure and the post-undressing blood pressure is equal to or greater than a second threshold, alert information that prompts the user to refrain from immersion in water. For example, the second threshold is 30 mmHg.

This makes it possible to properly notify the user of the potential risk of bathing or immersion in water.

In some embodiments, the undressing condition determiner determines, as the first time period, a period of time in the receive signal during which an area corresponding to a right shoulder of the user and an area corresponding to a left shoulder of the user simultaneously show movement speeds equal to or greater than the first threshold.

As a result, a period of time during which the user performs an undressing movement of pulling off clothing such as a sweater over the head is determined as the first time period. Performing this type of movement tends to cause a significant drop in the sensible temperature of the user. Thus, this type of movement is more likely to elevate blood pressure than other types of undressing movements. Accordingly, since alert information is presented based on the difference in blood pressure between the second time period and the third time period that immediately precede and immediately follow the first time period mentioned above, respectively, important information about potential risks can be presented to the user.

In some embodiments, the undressing condition determiner determines, as the first time period, a period of time in the receive signal during which an area corresponding to a central part of an upper body of the user shows a movement speed equal to or higher than the first threshold.

As a result, a period of time during which the user performs movement B, such as taking off of clothing, for example, a shirt by unfastening buttons on the upper central portion of the shirt, is determined as the first time period. Therefore, when this type of movement occurs in the first time period, appropriate alert information can be presented based on the difference in blood pressure between the second time period and the third time period that immediately precede and immediately follow the first time period, respectively.

In some embodiments, the alert information presenting apparatus further includes a pulse wave sensor that detects a pulse wave of the user, and the pulse wave acquirer acquires the pre-undressing pulse wave timing for the second time period and the post-undressing pulse wave timing for the third time period by using a signal indicative of the pulse wave detected by the pulse wave sensor.

This configuration allows pulse wave timing to be acquired without having an image of the user captured, thus protecting the privacy of the user.

In some embodiments, the undressing condition determiner further includes an undressing-progression determiner to recognize that the user has stopped undressing when the area corresponding to the right shoulder of the user and the area corresponding to the left shoulder of the user simultaneously show movement speeds equal to or greater than the first threshold and then the movement speeds become zero before movement is made past areas corresponding to both the right shoulder and the left shoulder of the user.

In some embodiments, the alert information presenting apparatus further includes an eyeglass donning and doffing determiner, and whether the user is wearing eyeglasses is determined by using the image acquirer, and when the user removes the eyeglasses, the signal acquirer and the pulse wave acquirer are activated, and an undressing condition is determined by using the undressing condition determiner.

In some embodiments, the alert information presenting apparatus further includes a dressing condition determiner, a dressing condition of the user after bathing is determined, blood pressures before and after the user dresses are measured by using the blood pressure calculator, entry of the user into a changing room after bathing is recognized by using the signal acquirer, and a blood pressure at a first time representing a time of the entry is measured by using the blood pressure calculator.

Hereinafter, embodiments will be described with reference to the drawings.

Embodiments described below each represent a general or specific example. Specific details set forth in the following description of embodiments, such as numeric values, shapes, materials, components, the arrangements and connections of components, steps, the order of steps, are for illustrative purposes only and not intended to limit the scope of the present disclosure. Those components in the following description of embodiments which are not cited in the independent claim representing the most generic concept of the present disclosure will be described as optional components.

### Embodiment 1

The following describes an alert information presenting apparatus according to Embodiment 1 that measures blood pressure in a contactless manner, and presents alert information based on the measurement results.

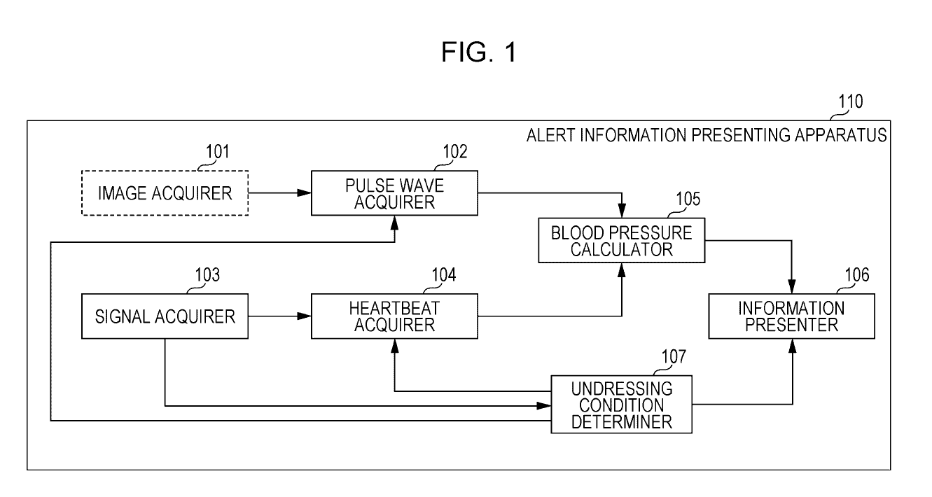

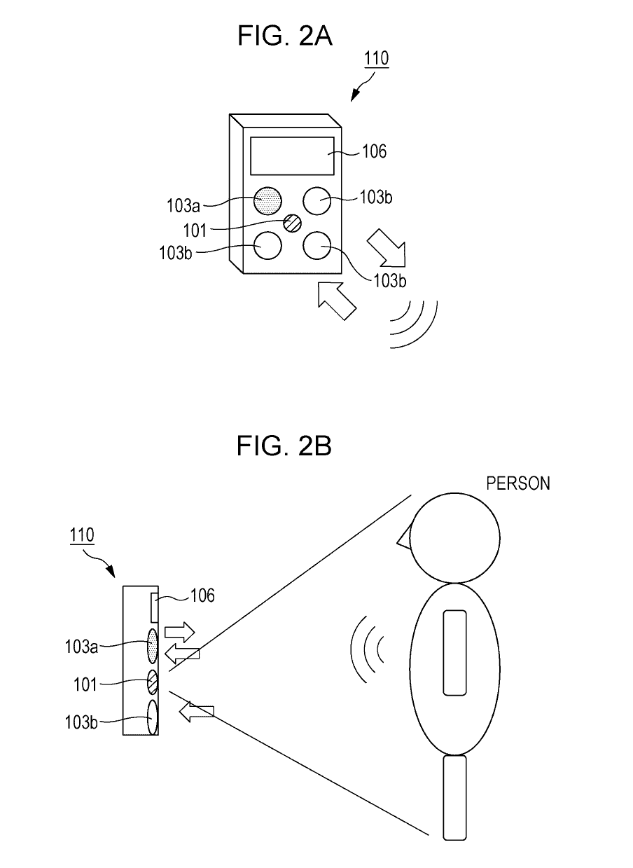

An alert information presenting apparatus 110 according to Embodiment 1 will be described below. FIG. 1 is a block diagram illustrating the configuration of the alert information presenting apparatus according to Embodiment 1. FIG. 2A is an external view of the alert information presenting apparatus according to Embodiment 1. FIG. 2B schematically illustrates how the alert information presenting apparatus is used by the user according to Embodiment 1.

As illustrated in FIG. 1, the alert information presenting apparatus 110 includes an image acquirer 101, a pulse wave acquirer 102, a signal acquirer 103, a heartbeat acquirer 104, a blood pressure calculator 105, an information presenter 106, and an undressing condition determiner 107.

Specifically, the alert information presenting apparatus 110 includes the signal acquirer 103 that acquires a receive signal including a radio wave reflected by the user, the undressing condition determiner 107 that determines, by using the movement speed of the user determined by using the receive signal and indicating the speed of a movement performed by the user, a first time period indicating a period of time during which the user is undressing, a second time period immediately preceding the first time period and indicating a period of time before the user undresses, and a third time period immediately following the first time period and indicating a period of time after the user undresses, the heartbeat acquirer 104 that acquires, by using the receive signal, a pre-undressing heartbeat timing and a post-undressing heartbeat timing, the pre-undressing heartbeat timing indicating a heartbeat timing in the second time period, the post-undressing heartbeat timing indicating a heartbeat timing in the third time period, the pulse wave acquirer 102 that acquires a pre-undressing pulse wave timing and a post-undressing pulse wave timing, the pre-undressing pulse wave timing indicating a pulse wave timing in the second time period, the post-undressing pulse wave timing indicating a pulse wave timing in the third time period, the blood pressure calculator 105 that calculates a pre-undressing blood pressure of the user and a post-undressing blood pressure of the user, the pre-undressing blood pressure being calculated by using a time difference between the pre-undressing heartbeat timing and the pre-undressing pulse wave timing, the post-undressing blood pressure being calculated by using a time difference between the post-undressing heartbeat timing and the post-undressing pulse wave timing, and the information presenter 106 that presents the user with alert information based on the difference between the pre-undressing blood pressure and the post-undressing blood pressure. The alert information presenting apparatus 110 according to Embodiment 1 further includes the image acquirer 101 that acquires an image including the skin of the user. Although the pulse wave acquirer 102 acquires the pulse wave timing by using the image, the pulse wave acquirer 102 may alternatively acquire the pulse wave timing by using information other than the image. That is, the alert information presenting apparatus 110 may not include the image acquirer 101. Further, the image acquirer 101 may either acquire the image through image capture or acquire the image not through image capture but from an apparatus located outside the alert information presenting apparatus 110. The above-mentioned functional blocks illustrated in FIG. 1 will be described later in detail.

FIG. 2A illustrates an example of the outside shape of the alert information presenting apparatus 110. As illustrated in FIG. 2A, the alert information presenting apparatus 110 has a housing in which the components illustrated in FIG. 1 are disposed.

The alert information presenting apparatus 110 has the following components disposed on the front of the housing: a transmitter 103a for transmitting millimeter wave radiation, three receivers 103b for receiving millimeter wave radiation, the image acquirer 101 implemented as, for example, a camera, and the information presenter 106 that presents alert information. The transmitter 103a and the three receivers 103b are included in the signal acquirer 103.

FIG. 2B illustrates how a person or person's movement is detected by using a millimeter wave. A millimeter wave transmitted by the transmitter 103a for transmitting millimeter wave radiation is reflected by a person, and the reflected signal is received on each of the receivers 103b to allow detection of the person or person's movement. According to the present disclosure, the user's movement associated with undressing, and pulsation caused by contraction of the heart are detected based on a signal reflected back from the person's chest. Further, an image of a person's body part such as a face or hand is captured by the image acquirer 101, and pulse is detected based on, for example, changes in luminance. Then, blood pressure is estimated based on the time difference (pulse transit time) between each beat and pulse, and the estimated blood pressure is presented on the information presenter 106. Further, a determination of whether to permit bathing is made based on variations in the blood pressure, and the result of this determination is also presented on the information presenter 106. Hereinafter, each of these components and details of the present disclosure will be described.

### Signal Acquirer 103

Referring back to FIG. 1, the signal acquirer 103 acquires a receive signal including a radio wave reflected by the user. That is, the signal acquirer 103 is a circuit that acquires, as a receive signal, a millimeter-wave signal reflected from a person. The signal acquirer 103 may acquire a millimeter-wave signal by receiving a millimeter wave, or may acquire a millimeter-wave signal by receiving the data of a millimeter-wave signal received by, for example, another apparatus.

If adapted to receive a millimeter wave, the signal acquirer 103 is implemented by a radar transceiver circuit that uses the millimeter band. Specifically, the signal acquirer 103 has the transmitter 103a (transmitting circuit) for transmitting millimeter wave radiation, and the receivers 103b (receiving circuits) for receiving millimeter wave radiation. The signal acquirer 103 determines the distance to a target based on the time difference between the time at which a transmit wave is transmitted, and the time at which the reflection of the transmit wave bounced off from the target is received. Further, the signal acquirer 103 determines the movement or speed of the target based on the phase or frequency difference between the transmit wave and the reflected wave. Further, the signal acquirer 103 determines the distance to an object based on the difference of the direction of arrival using an array antenna. Various known techniques exist for millimeter wave radars, such as those for so-called automobile collision avoidance, and those using code modulation aimed for higher accuracies, and no limitation is placed herein on their specific details.

The phase of a millimeter wave acquired by the signal acquirer 103 varies with a change in the distance between the signal acquirer 103 and a person caused by pulsation of the person's heart. Thus, if the signal acquirer 103 acquires millimeter waves successively, the acquired millimeter waves contain time-series information about changes in the distance between the signal acquirer 103 and the person.

The same process as mentioned above can be also executed by using radio wave bands different from the millimeter band. In that case, instead of the signal acquirer 103, a radio wave acquirer may be used that executes the same process as mentioned above by using radio wave bands different from the millimeter band.

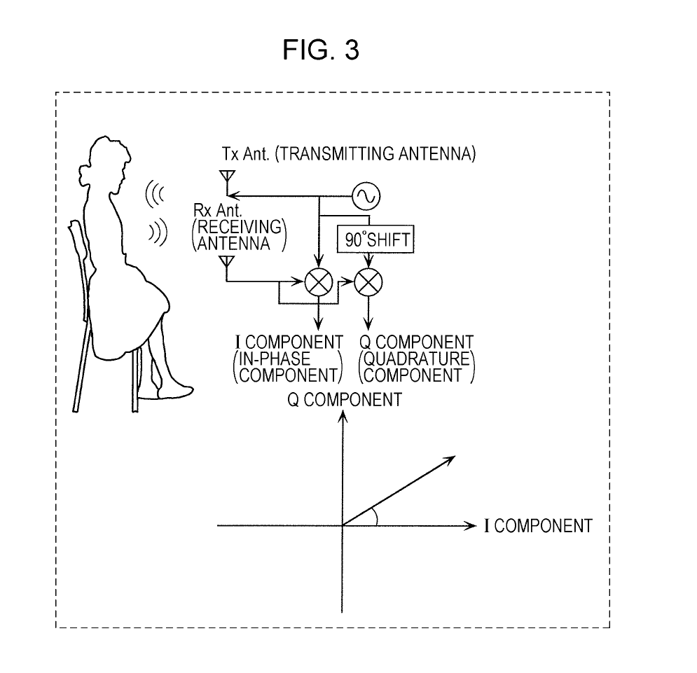

FIG. 3 illustrates the principle for calculation of heartbeat timing according to Embodiment 1.

In the signal acquirer 103, a predetermined frequency signal or pulse wave is transmitted by the transmitter 103a, and a signal bounced off a reflecting object is received by each of the receivers 103b. The signal transmitted at this time is modulated by, for example, frequency, amplitude, or code so that the timing when the signal is transmitted can be identified. Distance is measured by using the time difference of arrival. In Embodiment 1, for example, changes in the movement of the chest caused by contraction or pulsation of the heart are detected by using the reflections of millimeter waves bounced off the chest of a person. Movements such as those caused by heartbeat or respiration are very small. Accordingly, a change in distance is determined by using the phase difference between signals, in addition to the time difference of arrival. Letting the bounced-off signal be represented by r(t), distance is calculated by using Equations (1) to (5) below.

r(t)=A(t)cos(2πf₀(t−2d/c)) Eq. (1)

The in-phase component (I) and quadrature component (Q) of this signal are respectively represented as Equation (2) and Equation (3), and phase is calculated by Equation (4).

I(t)=A(t)cos(4πf₀d/c)) Eq. (2)

Q(t)=A(t)sin(4πf₀d/c)) Eq. (3)

phase=(4πf₀d/c))=tan⁻¹(Q/I) Eq. (4)

Distance is calculated by Equation (5) based on parameters such as the phase calculated by Equation (4), the round-trip travel distance of the signal, 2d, the speed of light, c, and wavelength, 2πf.

d=c/4πfₒ tan⁻¹(Q/I) Eq. (5)

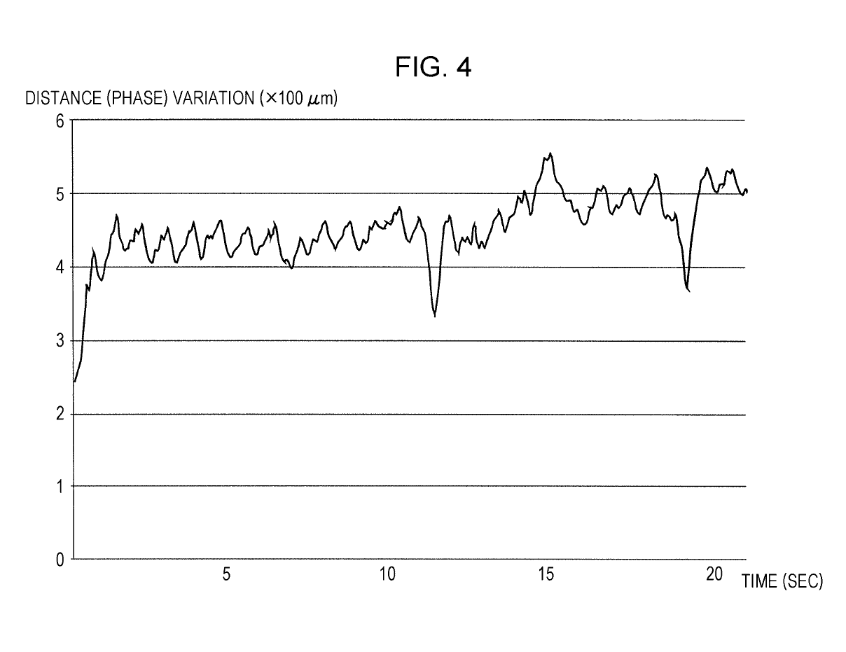

FIG. 4 illustrates changes in the movement (distance) of the chest obtained from a receive signal according to Embodiment 1.

FIG. 4 illustrates changes in the phase of waves reflected back from the chest of a person, which are obtained by using a spread spectrum radar using the 26-GHz band. A spread spectrum radar allows the relationship between distance resolution and maximum detectable range to be set flexibly by adjustment of the chip rate and code period of the pseudo-noise (PN) code. Thus, weak changes of the chest can be detected by adjusting the radar to the range of the chest, for example. In FIG. 4, the horizontal axis represents time, and the vertical axis represents phase shift. It can be appreciated from FIG. 4 that distance varies with each heartbeat representing contraction of the heart.

### Undressing Condition Determiner 107

The undressing condition determiner 107 identifies the location of a target user based on the distance information of a receive signal acquired by the signal acquirer 103. Further, the undressing condition determiner 107 calculates the movement speed of the user based on changes in the identified location of the target. That is, the movement speed of the user is calculated by using a receive signal. The undressing condition determiner 107 uses the movement speed to determine the first time period, the second time period, and the third time period. The first time period indicates a period of time during which the user is undressing. Specifically, the first time period is a period of time during which the movement speed is equal to or greater than a first threshold. The second time period, which immediately precedes the first time period, indicates a period of time before the user undresses. Specifically, the second time period is a period of time immediately preceding the first time period and during which the movement speed is less than the first threshold. The third time period, which immediately follows the first time period, indicates a period of time after the user undresses. Specifically, the third time period is a period of time immediately following the first time period and during which the movement speed is less than the first threshold.

To determine the first time period, the undressing condition determiner 107 identifies, for example, the right shoulder area and left shoulder area of the user. Then, the undressing condition determiner 107 calculates the movement speeds of those areas, and determines, as the first time period, a period of time during which those areas simultaneously show movement speeds equal to or greater than the first threshold. Alternatively, the undressing condition determiner 107 identifies the central area of the user's upper body. Then, the undressing condition determiner 107 calculates the movement speed of the central area, and determines, as the first time period, a period of time during which the central area shows a movement speed equal to or greater than the first threshold.

The undressing condition determiner 107 according to Embodiment 1 mentioned above will be described in more detail below.

The undressing condition determiner 107 determines undressing condition of the user by using a receive signal acquired by the signal acquirer 103.

The alert information presenting apparatus 110 according to Embodiment 1 aims to prevent so-called “heat shock” (adverse effects on the body caused by sudden temperature changes) during bathing due to increases in blood pressure associated with undressing, and thus focuses attention on blood pressures before and after undressing movement.

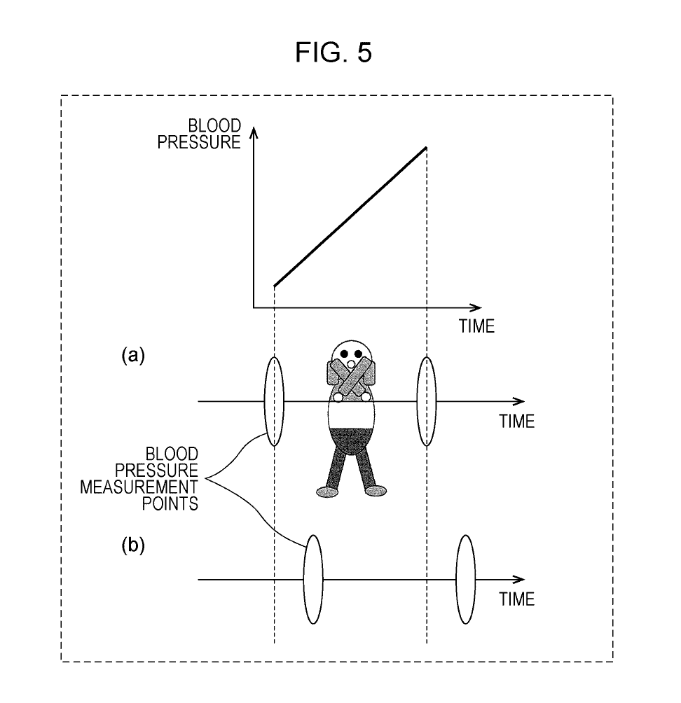

FIG. 5 illustrates the relationship between blood pressure and time points at which blood pressure is measured.

For example, as illustrated in FIG. 5(a), the alert information presenting apparatus 110 determines a user's movement by using the movement speed mentioned above. That is, the undressing condition determiner 107 determines whether an undressing movement has been performed by the user, or what kind of undressing movement has been performed by the user. Such a determination will be hereinafter referred to as determination of undressing condition, determination of undressing movement, or undressing determination. Further, the terms “undressing condition” and “undressing movement” are used synonymously in Embodiment 1.

As illustrated in FIG. 5(a), the alert information presenting apparatus 110 measures blood pressure while the user is stationary before undressing activity takes place. Blood pressure is measured based on pulse transit time, which is the difference between the timing of heartbeats and the timing of pulse waves described later. After it is determined that the user has executed an undressing movement, the alert information presenting apparatus 110 measures blood pressure once again when the user is stationary after the undressing movement. If an elevation in blood pressure is observed at this time as illustrated in FIG. 5, the alert information presenting apparatus 110 determines that the blood pressure elevation is caused by the undressing movement. In this case, for example, the alert information presenting apparatus 110 presents alert information to the user indicating that bathing is dangerous in the current situation.

In this regard, if blood pressure is measured without performing undressing determination, this means that undressing condition has not been determined. Thus, as illustrated in FIG. 5(b), the time point at which the measurement is taken may not necessarily coincide with the timing before undressing or after undressing. Further, even if an elevation in blood pressure is observed between any two given time points, it is not possible to determine whether the elevation in blood pressure is caused by undressing movement, making it impossible to provide an appropriate alert.

Accordingly, in the alert information presenting apparatus 110 according to Embodiment 1, undressing determination is performed by the undressing condition determiner 107 to correctly calculate blood pressure variation caused by undressing movement, and alert information is presented based on the blood pressure variation.

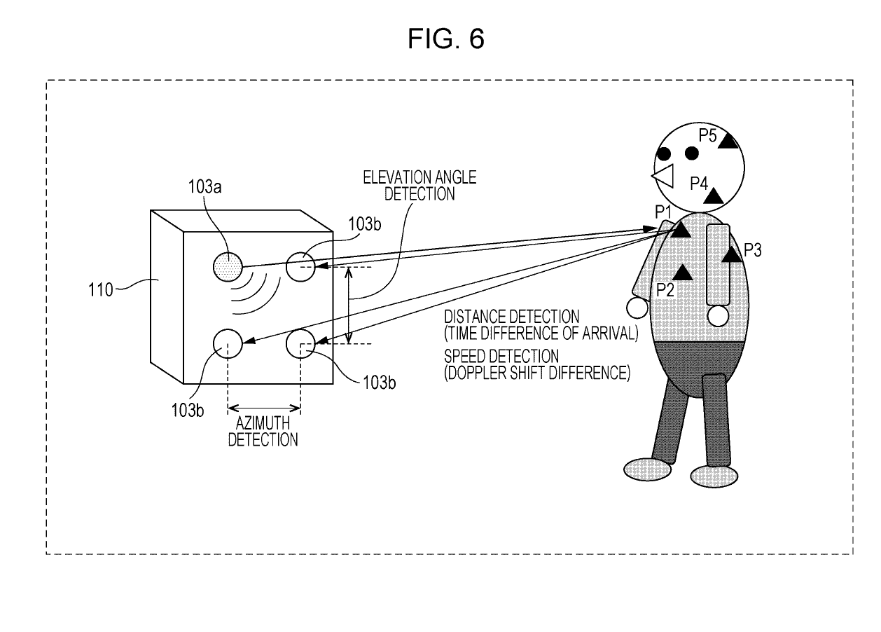

FIG. 6 illustrates how the undressing condition determiner 107 detects, by using a receive signal acquired by the signal acquirer 103, the presence of the user and the movement speed of the user.

The signal acquirer 103 includes three receivers 103b. The undressing condition determiner 107 performs azimuth and elevation angle detection, distance detection, and speed detection based on a receive signal acquired by each of the receivers 103b. Azimuth detection and elevation angle detection are performed by using the same principle. That is, from the distance d between two receivers 103b, and a phase difference φ indicated by the receive signal, the azimuth or elevation angle θ can be determined as Equation (6) below:

$$θ = {sin}^{- 1} ( \frac{φ λ}{2 π d} ) \qquad Eq . ( 6 )$$

where λ is the wavelength of the radio wave.

Further, distance detection and speed detection are performed by using a single receiver 103b. That is, the distance L is calculated as Equation (7) below by using the time difference between the transmit wave and the receive wave to and from the target (user):

$$L = \frac{c τ}{2} \qquad Eq . ( 7 )$$

where c is the speed of light.

Further, the movement speed v is calculated from the difference in frequency between the transmit wave and receive wave as Equation (8):

$$v = \frac{f - {f}_{0}}{{f}_{0}} C \qquad Eq . ( 8 )$$

where f is the frequency of the receive wave, and f₀ is the frequency of the transmit wave.

The undressing condition determiner 107 executes the azimuth and elevation angle detection and the distance detection to identify body parts (for example, body parts P1 to P5 illustrated in FIG. 6) of the user, and detects the speeds of the identified body parts. The undressing condition determiner 107 thus determines user's undressing condition (undressing movement).

As illustrated in FIG. 2A and FIG. 6, the alert information presenting apparatus 110 includes a plurality of receivers 103b for receiving millimeter wave radiation. Specifically, the alert information presenting apparatus 110 includes a set of two receivers 103b in the vertical direction, and a set of two receivers 103b in the horizontal direction orthogonal to the vertical direction. One of the receivers 103b included in each of the two sets is shared between the two sets. Thus, the alert information presenting apparatus 110 has a total of three receivers 103b. This is to calculate, by using the time difference of arrival (phase difference) of reflected waves, the angles of arrival (azimuths and elevation angles) of the reflected waves as mentioned above, and calculates XY-coordinates (coordinates each made up of value X on the horizontal axis and value Y on the vertical axis) in the space of the user representing a reflecting object. In Embodiment 1, one set of two receivers 103b exists in the vertical direction, and one set of two receivers 103b exists in the horizontal direction, with one receiver 103b included in each of the two sets being shared between the two sets, and hence a total of three receivers 103b exist. However, this is not to be construed in a limiting sense. For example, the alert information presenting apparatus 110 may include a plurality of receivers 103b disposed along a given direction, and a plurality of receivers 103b disposed along one direction orthogonal to this direction. Further, a plurality of receivers 103b may be arranged in a given direction, and the receivers 103b may be provided with directivity.

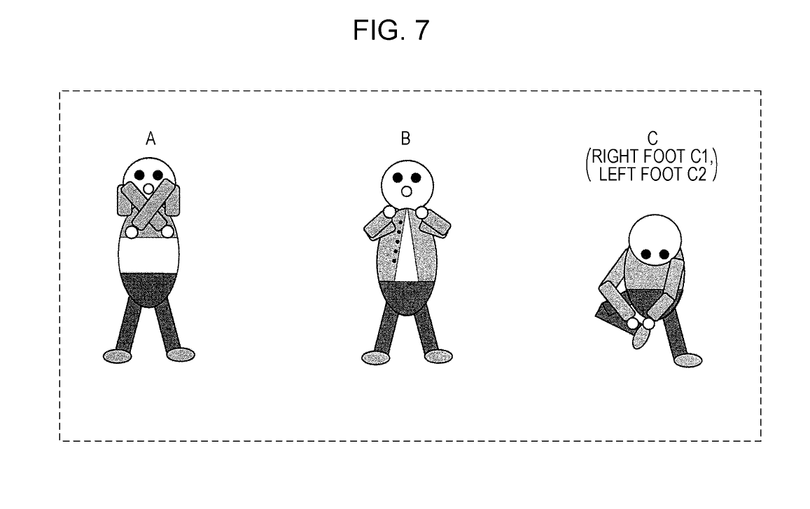

FIG. 7 illustrates an example of user's undressing conditions (undressing movements) determined by using millimeter waves. Movement A represents undressing performed by the user by pulling clothing over the head. A characteristic feature of movement A, which is typically observed when, for example, a person takes off a sweater or shirt, is that both arms or both hands are moved to positions above the shoulders. Movement A involves taking off a heavy clothing item, or taking off underwear that results in the user's skin being directly exposed to the outside air, causing a sharp drop in the sensible temperature of the user. Therefore, a characteristic feature of movement A is that this type of movement is more likely to elevate blood pressure than other types of undressing movements.

Movement B represents undressing from the shoulders down, by moving the hands from the central part of the upper body toward the shoulders. A characteristic feature of movement B, which is typically observed when a person takes off a shirt, a cardigan, a coat, or other items of clothing, is that undressing proceeds in order beginning with the shoulders. Movement C represents undressing performed with one foot raised. A characteristic feature of movement C is that this movement always takes place when a person takes off items of clothing on the lower body, such as pants or socks. Since movement C takes place on both the left and right sides of the body, the undressing movement for the right side is denoted by C1, and the undressing movement for the left side is denoted by C2.

Although various movements are categorized according to the type of associated clothing, this is not to be construed in a limiting sense. For example, taking off of a shirt is represented by movement B in the above example. However, even if a shirt is the item of clothing to be taken off, the user may sometimes undress by unfastening buttons on the upper part, and then pulling the shirt over the head like a sweater. In this case, the undressing movement may be determined as movement A even though the user is taking off a shirt.

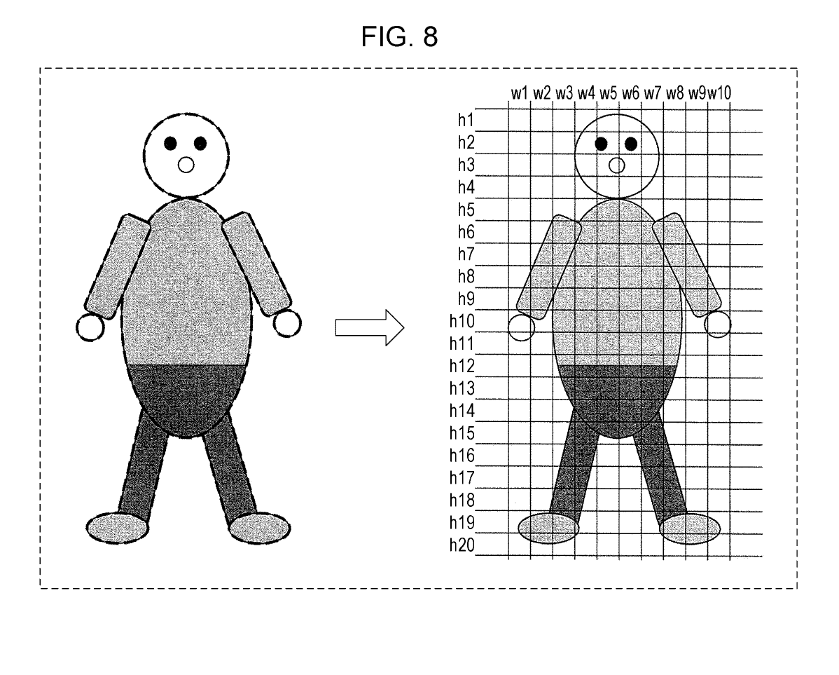

FIG. 8 illustrates a method for obtaining information about the user's body from azimuth, elevation angle, and distance detected by using the signal acquirer 103. First, the undressing condition determiner 107 accumulates data including the azimuth, elevation angle θ, and distance L of each body part, based on receive signals acquired by the signal acquirer 103 and representing waves reflected from the user's body or its nearby areas.

The transmitter 103a of the signal acquirer 103 transmits millimeter waves with uniform frequency toward the entirety of a target. Each of the receivers 103b of the signal acquirer 103 detects the frequency and phase of the corresponding millimeter-wave signal received. The azimuth and elevation angle of the target are calculated by using the phase difference of signals received on the receivers 103b of the signal acquirer 103. A predetermined range is allowed for the azimuth and the elevation angle thus calculated, and based on distance, the frequency associated with movement is obtained for each area of the body. For example, for an elevation angle range between 0 degrees and 10 degrees and an azimuth range between 0 degrees and 10 degrees, the corresponding area is represented as (h10, w6), and for an elevation angle range between 10 degrees and 20 degrees and an azimuth range between 0 degrees and 10 degrees, the corresponding area is represented as (h9, w6). In this way, speed variation can be obtained for each individual area of the body. As for the specific calculation method for each area, the calculation is performed by using Equations (6), (7), and (8). The size of each area is determined by using information on the user's body height and breadth. The specific method for calculating information on the user's body height and breadth will be described later.

In this way, each of the directions of received millimeter waves, and the frequency for that direction can be detected. Therefore, the motion of the target in each direction can be determined, thus allowing the location of the user's body to be determined.

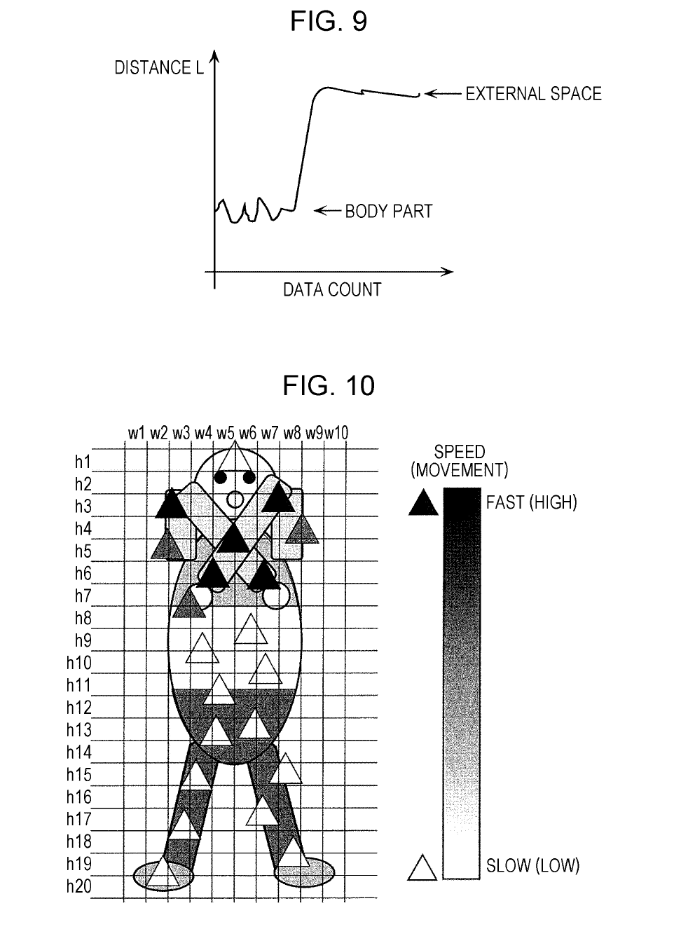

FIG. 9 illustrates a method for determining the presence of a body.

The distance L calculated based on a millimeter wave reflected by an area other than a human body is sufficiently greater than the distance L calculated based on a millimeter wave reflected by the human body. Accordingly, by checking the difference between the two values, the undressing condition determiner 107 is able to calculate the boundary between the human body and the external space, thus determining the presence of the human body. Although a human body still exhibits very weak body movements even under resting conditions, a nearby obstacle is a solid matter, which exhibits no oscillations. Accordingly, the undressing condition determiner 107 may determine a body part and the external space based on changes in the distance L over time.

By using the distance L to each body part, and the elevation angle θ obtained at the same time, the undressing condition determiner 107 can calculate the height of each corresponding point on the user from the ground by the following equation: y=L·sinθ+z, where z is the height of the signal acquirer 103 from the ground. Further, based on the same principle, the undressing condition determiner 107 can calculate the breadth of the user by using the azimuth and the distance L.

The maximum value of the obtained height y represents the body height of the user. As illustrated in FIG. 8, the undressing condition determiner 107 segments the body into parts by dividing the body height into twenty equal parts (h1 to h20) and the breadth into ten equal parts (w1 to w10), thus allowing each body part to be represented by coordinates such as (w4, h8).

For example, suppose that the reflected wave from the body contains the following information: the distance L is in the range between 50 cm and 140 cm, and z is 30 cm. In this case, the maximum value, Lmax, of the distance L used for calculating body height is obtained as Lmax=140 cm. If the elevation angle at this time is +45 degrees, the body height can be determined as 170 cm from the equation y=L·sinθ+z. Likewise, if the breadth is 50 cm, then each of the areas represented in coordinates has a size of 8.5 cm×5 cm. Suppose that, for example, the following pieces of information are obtained in this state from the reflected wave: L=100 cm, elevation angle=+60 degrees, azimuth=+10 degrees, and speed=20 cm/s. In this case, the undressing condition determiner 107 determines that this reflected wave is the reflection from the area (w4, h14), and that the body part located in this area is moving at a speed of 20 cm/s. In the following description of undressing movements, each area of the body necessary for explanation will be designated by the following notation: (number N of area w, number M of area h).

FIG. 10 illustrates a method of determining an undressing movement performed by the user. The undressing condition determiner 107 uses information about various areas of the user's body identified as mentioned above to find how movement speed in each area is changing with time. In FIG. 10, triangles that appear blacker with increasing movement speed in an area of interest are displayed in the corresponding area and, conversely, triangles that appear whiter with increasing movement speed in an area of interest are displayed in the corresponding area.

The undressing movement illustrated in FIG. 10 is movement A. At this time, both hands of the user move from the front of the body to positions above the shoulders. That is, both hands are initially located in the area (w3, h10)-(w8, h10), and subsequently move with time to the area (w4, h5)-(w7, h5). Accordingly, the movement speed is higher in these areas than in other areas. Each of areas represented in the from (wN1, hM1)-(wN2, hM2) such as (w3, h10)-(w8, h10) refers to a rectangular area with the line connecting between (wN1, hM1) and (wN2, hM2) as the diagonal.

Normally, movement A does not accompany simultaneous movement of lower body parts such as the feet and waist. Thus, the speed of movement in the area (w1, h12)-(w10, h20) is zero or lower than a predetermined value. Accordingly, if the speed of movement in a specific area (w3, h5)-(w8, h8) is equal to or greater than a predetermined threshold, and the speed of movement in the area (w1, h12)-(w10, h20) is equal to or lower than a predetermined value, the undressing condition determiner 107 determines that the user is performing movement A as an undressing movement.

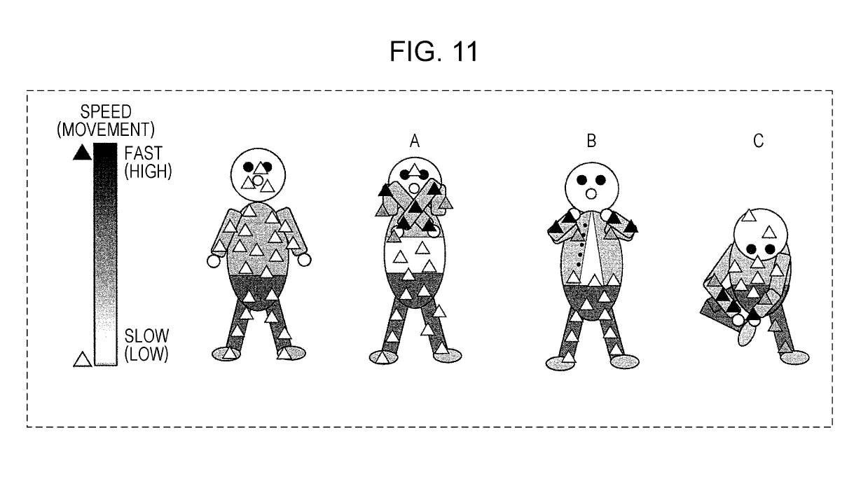

FIG. 11 illustrates patterns of movement speeds in various areas used to determine various undressing movements.

In movement A, the movement speeds of the left and right shoulders of the user are both greater than the movement speeds of other body parts. Accordingly, the undressing condition determiner 107 may determine that the user is performing movement A as an undressing movement if both the user's right shoulder area and the user's left shoulder area simultaneously show movement speeds equal to or greater than the first threshold in the receive signal.

In movement B, the movement speed of the central part of the user's upper body is greater than the movement speeds of body parts other than the central part of the upper body. Accordingly, the undressing condition determiner 107 may determine that the user is performing movement B as an undressing movement if the central area of the user's upper body shows a movement speed equal to or greater than the first threshold in the receive signal.

In movement C, the movement speed of the right side or left side of the user's lower body is greater than the movement speeds of other body parts. Accordingly, the undressing condition determiner 107 may determine that the user is performing movement C as an undressing movement if the right side or left side area of the user's lower body shows a movement speed equal to or greater than the first threshold in the receive signal.

Accordingly, the undressing condition determiner 107 determines the first time period indicating a period of time during which the user is undressing as follows. That is, the undressing condition determiner 107 determines, as the first time period, a period of time during which the receive signal shows a movement speed equal to or greater than the first threshold. That is, the undressing condition determiner 107 determines, as the first time period indicating a time period during which the user is performing movement A, a period of time in the receive signal during which both the user's right shoulder area and the user's left shoulder area simultaneously show movement speeds equal to or greater than the first threshold. Further, the undressing condition determiner 107 may determine, as the first time period indicating a time period during which the user is performing movement B, a period of time in the receive signal during which the central area of the user's upper body shows a movement speed equal to or greater than the first threshold. Further, the undressing condition determiner 107 may determine, as the first time period indicating a time period during which the user is performing movement C, a period of time in the receive signal during which the right side or left side area of the user's lower body show a movement speed equal to or greater than the first threshold. Conversely, the undressing condition determiner 107 determines, as each of the second time period before undressing and the third time period after undressing, a period of time during which the receive signal shows a movement speed less than the first threshold. More specifically, the undressing condition determiner 107 determines, as the second time period before undressing, a period of time immediately preceding the first time period. Further, the undressing condition determiner 107 determines, as the third time period after undressing, a period of time immediately following the first time period.

Although the undressing condition determiner 107 determines, as the first time period, a period of time during which the receive signal shows a movement speed equal to or greater than the first threshold, this is not to be construed in a limiting sense. For example, even if the time during which the signal shows a movement speed equal to or greater than the first threshold is non-continuous, the non-continuous time period may be included in the first time period. Such a non-continuous time period may be set as a time period in which the length of time during which the receive signal shows a movement speed below the first threshold is less than a predetermined value, for example, 10 seconds. For example, the following case is considered. After the user is detected, a receive signal that shows a movement speed less than the first threshold is detected continuously for 30 seconds (=Period A). Immediately after this period, a receive signal that shows a movement speed equal to or greater than the first threshold is detected continuously for 20 seconds (=Period B). Immediately after this period, a receive signal that shows a movement speed less than the first threshold is detected continuously for 5 seconds (=Period C). Immediately after this period, a receive signal that shows a movement speed equal to or greater than the first threshold is detected continuously for 50 seconds (=Period D). Immediately after this period, a receive signal that shows a movement speed less than the first threshold is detected continuously for 45 seconds (=Period E). In this case, the undressing condition determiner 107 may determine the second time period and the third time period as follows with reference to the first time period (=Period B+Period C+Period D) including the time period in which a receive signal that shows a movement speed equal to or greater than the first threshold is first detected after the detection of the user. That is, the time period (=Period A) with a movement speed less than the first threshold that immediately precedes the first time period is determined as the second time period, and the time period (=Period E) with a movement speed less than the first threshold that immediately follows the first time period is determined as the third time period.

Although the undressing condition determiner 107 uses the first threshold as the threshold for determining various undressing movements of the user in the above-mentioned example, this is not to be construed in a limiting sense. For example, to emphasize characteristics specific to each individual dressing movement, movement speed thresholds for various body parts of the user may be set for each individual undressing movement.

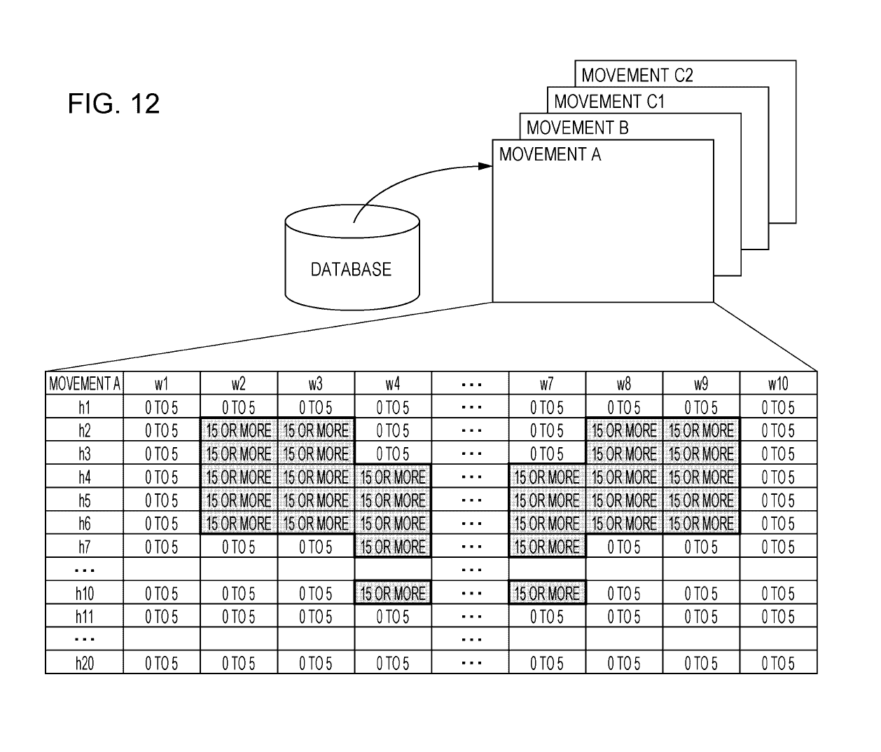

FIG. 12 illustrates an example of movement speed thresholds for various areas set for each individual undressing movement.

The undressing condition determiner 107 matches the speed of movement measured in each area against data (first threshold) in a database illustrated in FIG. 12 to determine undressing condition.

For example, in movement A representing undressing of a sweater or underwear, the user moves hands from the front of the body to positions above the shoulders. The average speed at which a human raises his or her arm is 20 cm/s. Accordingly, as illustrated in FIG. 12, the database for movement A shows 15 cm/s as the first movement speed threshold for a specific area located between the chest and the shoulders. Thus, if the movement speed in the specific area is equal to or greater than 15 cm/s, and the movement speed is changing with time, the undressing condition determiner 107 determines that the user is performing movement A as an undressing movement.

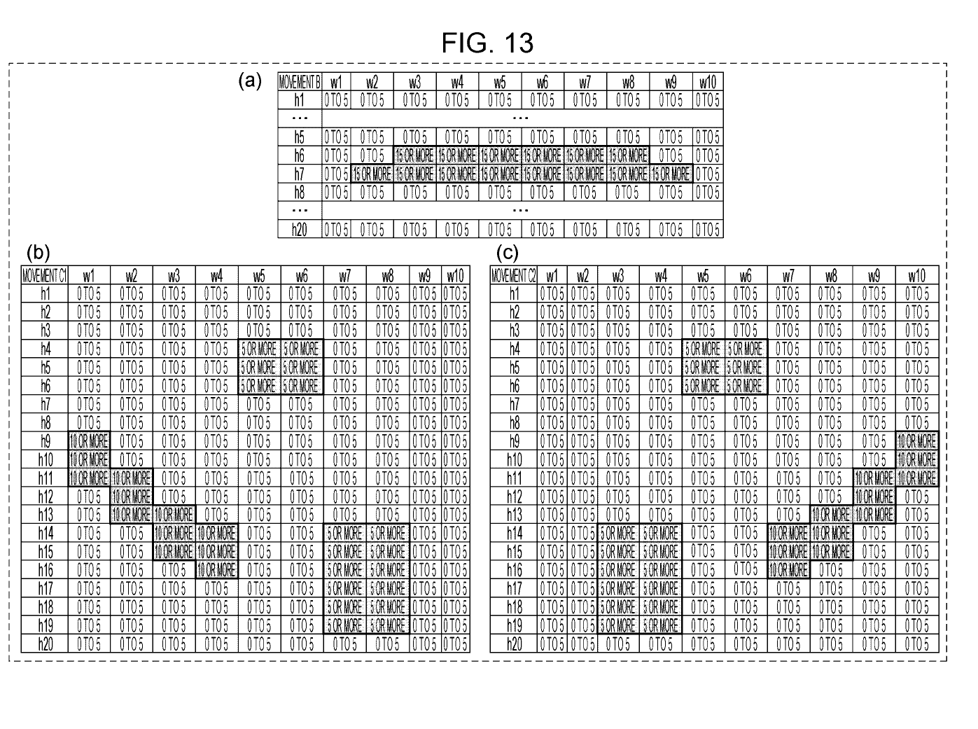

FIG. 13 illustrates movement speed thresholds for various areas in movement B and movements C1 and C2.

In movement B, although there are some temporal variations depending on whether the user undresses while leaving some buttons or the like fastened or after unfastening all of the buttons or the like, the user always starts to undress from the central part of the upper body. At this time, the speed at which the user's hands move from the central part of the upper body toward the shoulders is equal or greater than 15 cm/s. Accordingly, as illustrated in FIG. 13(a), the database shows 15 cm/s as the first movement speed threshold for the area of the central part of the user's upper body. Thus, if the movement speed in the central part is equal to or greater than 15 cm/s, the undressing condition determiner 107 determines that the user is performing movement B as an undressing movement.

In movement C, the movement speed of the area corresponding to the foot being undressed is greater than 10 cm/s, and the movement speed of the area corresponding to the other foot supporting the body, or the movement speed of the area corresponding to the head looking down on the foot is equal to or greater than 5 cm/s. Accordingly, as illustrated in FIG. 13(b) and FIG. 13(c), the database shows 10 cm/s as the first movement speed threshold for the area corresponding to the user's foot being undressed, and 5 cm/s as the first movement speed threshold for the area corresponding to the other foot or head. Thus, if the movement speed in the area corresponding to the foot being undressed is equal to or greater than 10 cm/s, and the movement speed of the area corresponding to the other foot or head is equal to or greater than 5 cm/s, the undressing condition determiner 107 determines that the user is performing movement C1 or C2 as an undressing movement.

It is to be noted when performing a check against the database that the same undressing movement is executed slightly differently by each individual person. Accordingly, the undressing condition determiner 107 may determine whether a match with a measured movement speed is obtained for the data of 80% of all of the areas (w1, h1) to (w10, h20) shown in the database. If such a match is determined to exist, the undressing condition determiner 107 determines that the user has executed an undressing movement (one of movements A to C) corresponding to the database.

Although a movement speed threshold for each individual area is set for each individual movement in the database, the threshold may not necessarily be set to such a value. The speed of undressing varies with each individual user. Hence, rather than setting a specific numeric value, an alternative approach may be to use, as a reference, the maximum speed at which each individual undressing movement is executed, determine a threshold as a ratio relative to this reference value, and store the determined threshold in the database. For example, suppose that, during movement A performed by a given user, the arm-raising movement is executed fastest, at a speed of 30 cm/s. Further, the database for movement C specifies a ratio of 2/3 as the threshold for the foot area. In this case, the undressing condition determiner 107 may determine that the average undressing speed of this user is high, and with the maximum speed of 30 cm/s as a reference, the movement speed threshold for the foot area in movement C may be calculated as 20 cm/s.

In Embodiment 1, undressing determination based on matching of a measured movement speed against data in the database is performed at one point in time during execution of a movement, for example, when the movement speed exceeds the first threshold in the second time period. However, this is not to be construed in a limiting sense. For example, the alert information presenting apparatus 110 may have a database with thresholds that change in time series while the corresponding movements are executed. In this case, the undressing condition determiner 107 may determine user's undressing condition by matching movement speeds measured at various points in time during execution of a movement against the thresholds in the database that change in time series.

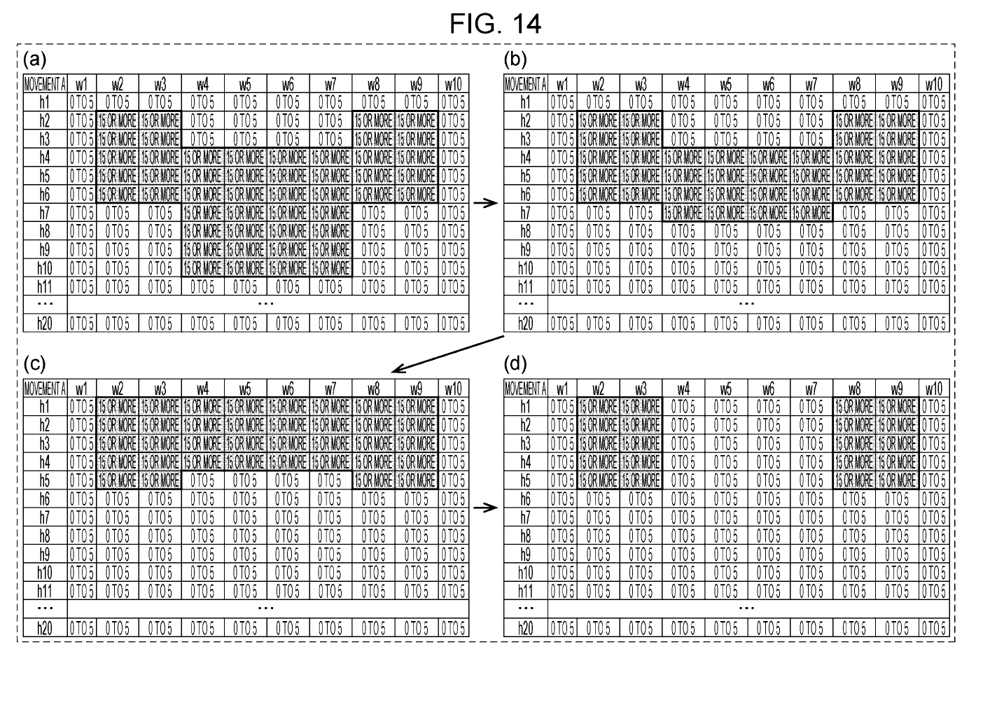

FIG. 14 illustrates an example of a database storing thresholds that change in time series. To represent a plurality of temporal state transitions occurring while a movement is executed, the database contains movement speed thresholds for various areas at each of four timings during the movement.

The database illustrated in FIG. 14 is a database for movement A. In movement A, the upper body moves at the first timing. Thus, as illustrated in FIG. 14(a), a large value is set as the movement speed threshold for areas located relatively high on the body shown in the database. At the next and subsequent timings, the user normally executes a movement of gradually raising both hands to take off the clothing on the upper body. Thus, as illustrated in FIG. 14(a) to FIG. 14(c), the areas with large movement speed threshold shifts gradually higher. At the next timing, the user finishes taking off clothing on the upper body, and thus both hands of the user move. As a result, the movement speed threshold for the body's central part shown in the database becomes smaller, with the movement speed threshold for the areas corresponding to both arms being maintained at a large value as illustrated in FIG. 14(d). As described above, the alert information presenting apparatus 110 has a database representing time-series transition data, and makes reference to this database to determine undressing movement, thus allowing undressing determination to be performed with improved accuracy.

Although the database used in the above-mentioned example has a predetermined threshold for the entire area corresponding to each body part, this is not to be construed in a limiting sense. For example, movement A typically involves the user moving mainly both hands simultaneously and at nearly the same height and speed. The resulting movement is thus often bilaterally symmetrical with respect to the center of the body. In contrast, during execution of a movement such as taking off clothing from one shoulder like movement B, movement first takes place from the center of the body toward one of the areas located on the left and right sides of the body, followed by movement in the other area. Further, in the case of a movement such as taking off clothing one foot at a time like movement C, movement first takes place in one area, followed by movement in the other area. For movement C, however, such areas with movement are located lower than the upper body area. Accordingly, for example, the undressing condition determiner 107 identifies the center of a body part, and determines whether changes in movement speed are being observed simultaneously in both left and right areas with respect to the center, and areas with high movement speed are shifting gradually higher. If such areas are determined to be shifting higher, then the undressing condition determiner 107 determines that the user is performing movement A (for example, taking off an item of outerwear with no buttons). When the undressing condition determiner 107 determines that such areas are not shifting higher, if changes in movement speed are not being observed simultaneously in both the left and right areas of the upper body, then the undressing condition determiner 107 determines that the user is performing movement B (for example, taking off an item of outerwear with buttons). Alternatively, if changes in movement speed are not being observed simultaneously in both the left and right areas of the lower body, then the undressing condition determiner 107 determines that the user is performing a movement of pulling down pants or a skirt. Further, if changes in movement speed are being observed in both the left and right areas of the lower body at different timings, then the undressing condition determiner 107 determines that the user is performing movement C.

As described above, each undressing movement includes a characteristic movement. Accordingly, undressing determination may be performed by matching such a characteristic movement against a movement identified based on a measured movement speed. This allows for robust and accurate determination.

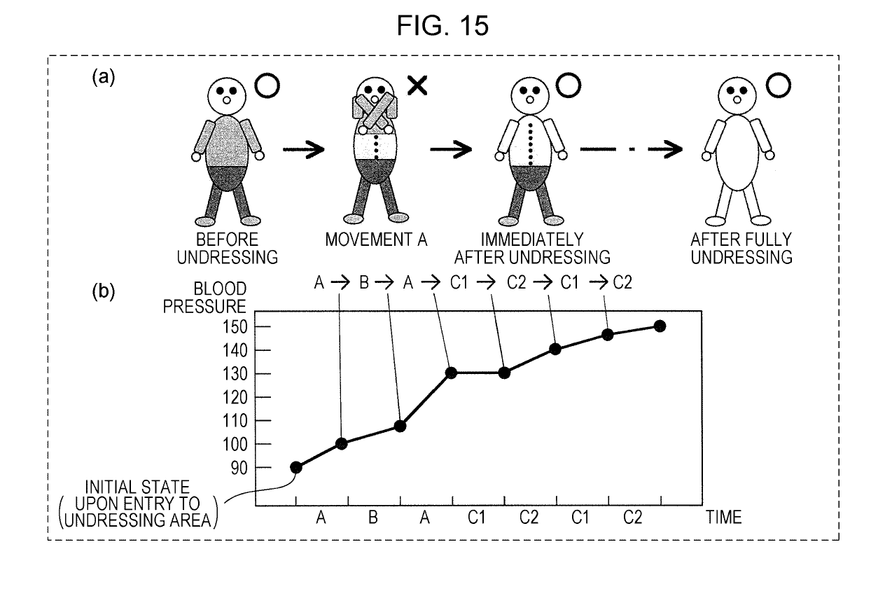

FIG. 15 illustrates undressing determination and the timing of blood pressure measurements.

In Embodiment 1, blood pressure is measured by using millimeter waves and images as will be described later. This means that blood pressure can be measured with greater accuracy if there is relatively little body movement during the measurement. Accordingly, the alert information presenting apparatus 110 according to Embodiment 1 uses the movement speed of the user to determine the first time period indicating a period of time during which the user is undressing, the second time period indicating a period of time before the user undresses, and the third time period indicating a period of time after the user undresses. This allows the alert information presenting apparatus 110 to measure blood pressure after completion of each undressing movement once the user becomes stationary or once there is relatively little body movement.

For example, as illustrated in FIG. 15(a), the alert information presenting apparatus 110 measures user's resting blood pressure both before undressing (second time period) and immediately after undressing (third time period) but does not measure blood pressure while an undressing movement such as movement A is performed (first time period). As a result, as illustrated in FIG. 15(b), the alert information presenting apparatus 110 performs blood pressure measurement at timings before and after undressing when there is relatively little body movement, as indicated by the points of filled circles. Then, the information presenter 106 determines whether to permit bathing based on changes in blood pressure (also referred to as blood pressure variation) before and after each undressing movement. The details of this determination performed by the information presenter 106 will be given later.

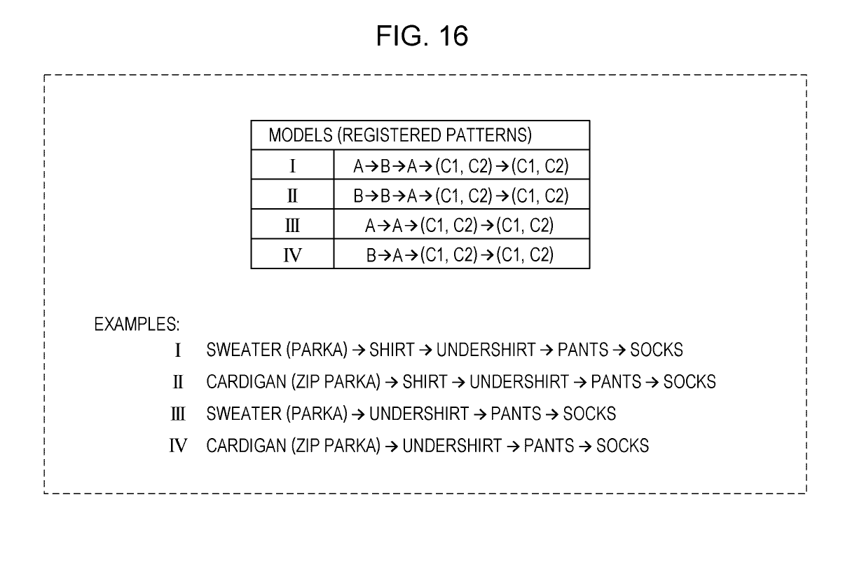

FIG. 16 illustrates patterns of user's undressing order. The undressing order refers to the order in which undressing movements are executed in time series. The alert information presenting apparatus 110 records each undressing movement performed by the user, and matches the pattern of the undressing order including such recorded undressing movements against a pattern registered in the database illustrated in FIG. 16 to determine whether the user has completed undressing.

For example, if the user commonly executes undressing according to registered pattern I in winter time, the undressing condition determiner 107 determines that the user has completed undressing when the last sock is taken off.

Such a pattern of undressing order varies with each individual user. Even for the same user, this pattern varies according to the clothes the user is wearing. Accordingly, day-to-day patterns of undressing order for each individual user may be accumulated in a database in advance, and data (undressing order) acquired for each season or each date may be matched against the database to determine the pattern of user's undressing order. At this time, the completion of undressing is determined as follows. That is, the undressing condition determiner 107 determines that undressing is completed if none of undressing movements A, B, and C is recognized or if no change in movement speed is detected for three minutes or more after the start of undressing is recognized.

Even after the undressing condition determiner 107 determines that undressing is completed, if it is confirmed through detection of movement speed that undressing movement A, B, or C has been executed, the undressing condition determiner 107 cancels the determination that undressing is completed. Then, the undressing condition determiner 107 may store the order of undressing including the undressing movement confirmed to have been executed, into the database as a new pattern of undressing order (registered pattern or model).

In this way, in Embodiment 1, undressing movements are finely classified to identify changes in blood pressure caused by each undressing movement. This makes it possible to determine whether such changes in user's blood pressure can potentially trigger dangerous conditions during or after bathing that takes place after undressing. For example, if blood pressure is elevated significantly by movement A, and then continues to rise as the user performs movement B and movement C, it can be determined that the blood pressure is rising as a result of a drop in the sensible temperature of the user caused by undressing. Therefore, taking a bath in that state is expected to cause a rapid drop in blood pressure that can lead to drowning, or cause rapid cooling of the body after bathing that can cause disorders such as myocardial infarction or cerebral stroke. Conversely, with a time-series observation of changes in blood pressure, it is difficult to identify the cause of the changes in blood pressure. If changes in blood pressure (such as changes in blood pressure from a threshold or blood pressure elevation difference) are used to determine whether to provide an alert about bathing, it is not possible to discern whether the elevated blood pressure is caused by a rise in blood pressure due to coldness, or is simply a result of hypertension. Therefore, it is not appropriate to determine whether to provide an alert about bathing in this way. In contrast, Embodiment 1 identifies changes in blood pressure associated with undressing, thus allowing an alert to be provided accurately, which proves very useful.

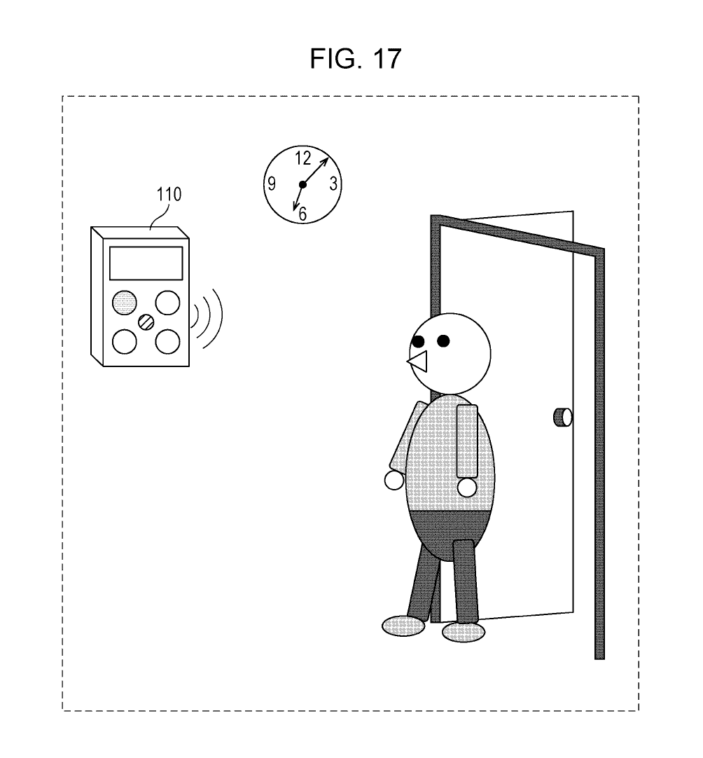

FIG. 17 illustrates blood pressure measurement performed before undressing. The undressing condition determiner 107 constantly determines whether the user is present in the changing room based on a receive signal acquired by the signal acquirer 103, and determines that blood pressure measurement is to be taken whenever the user enters the changing room. Alternatively, the undressing condition determiner 107 always determines whether the user is present in the vicinity of the alert information presenting apparatus 110 based on a receive signal acquired by the signal acquirer 103, and determines that blood pressure measurement is to be taken whenever the user comes in proximity to the alert information presenting apparatus 110. That is, the undressing condition determiner 107 determines the second time period. Based on this determination, the blood pressure calculator 105 calculates blood pressure. Once the start of undressing is detected, the undressing condition determiner 107 continuously determines undressing condition as mentioned above and, at the same time, the undressing condition determiner 107 determines to take a blood pressure measurement after the undressing movement or before the next undressing movement. That is, the undressing condition determiner 107 determines the second or third time period. Based on this determination, the blood pressure calculator 105 calculates blood pressure.

The undressing condition determiner 107 may record the time at which the user takes a bath (or the time when undressing starts) into a database every time such an event occurs. In this case, the undressing condition determiner 107 makes reference to the database to determine whether the current time is the time at which the user takes a bath. If the undressing condition determiner 107 determines that the current time is the time at which the user takes a bath, the undressing condition determiner 107 determines user's undressing condition based on a receive signal acquired by the signal acquirer 103.

### Heartbeat Acquirer 104

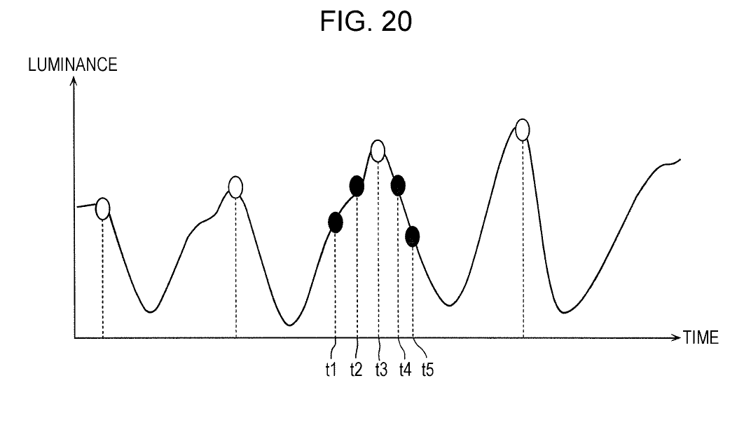

Referring back to FIG. 1, the heartbeat acquirer 104 uses a receive signal acquired by the signal acquirer 103 to acquire the pre-undressing timing of heartbeat for the second time period mentioned above and the post-undressing timing of heartbeat for the third time period mentioned above. Specifically, the heartbeat acquirer 104 calculates the timing of heartbeat (to be also referred to as “heartbeat timing” hereinafter) based on information about phase change. The heartbeat timing can be calculated by, for example, a predetermined peak search process as in the example illustrated in FIG. 20 described later. Specifically, for example, the timing of one heartbeat may be determined by identifying a given peak in the receive signal, and identifying the following peak within a period in the receive signal following the elapse of a predetermined length of time, for example, 333 ms to 1000 ms, from the given identified peak, and this process may be repeated to determine the timing of a plurality of heartbeats.

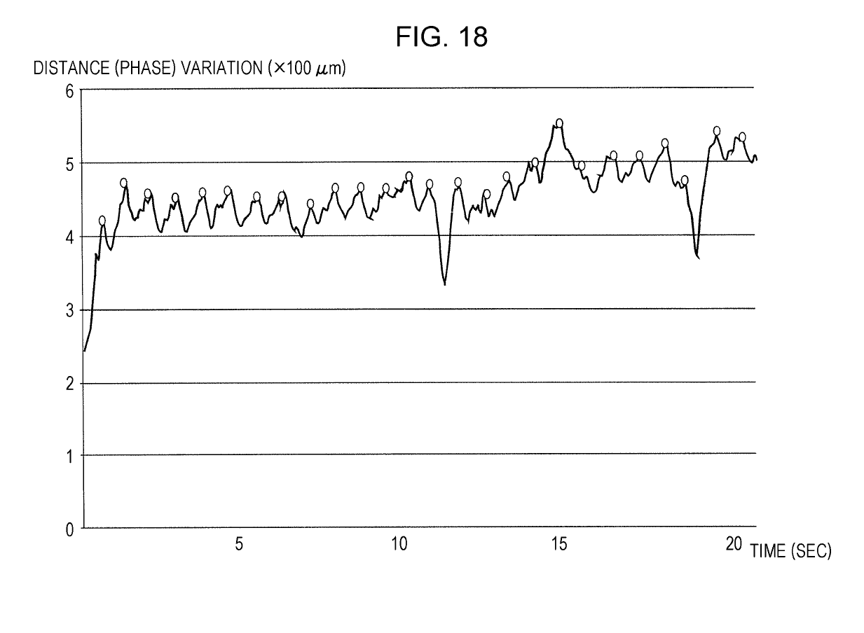

FIG. 18 illustrates an example of calculation of heartbeat timing according to Embodiment 1. Specifically, FIG. 18 illustrates heartbeat timing obtained by peak search. As in FIG. 4, the horizontal axis represents time, and the vertical axis represents phase change, with obtained peaks indicated by unfilled circles.

The heartbeat acquirer 104 acquires the heartbeat timing mentioned above before and after undressing. That is, the heartbeat acquirer 104 acquires, as the pre-undressing heartbeat timing, the time points corresponding to peaks of the time waveform in the second time period of the receive signal. Further, the heartbeat acquirer 104 acquires, as the post-undressing heartbeat timing, the time points corresponding to peaks of the time waveform in the third time period of the receive signal.

### Image Acquirer 101

The image acquirer 101 acquires an image including the user's skin. Specifically, the image acquirer 101 acquires a skin image, which is a captured image of the skin of a person including the face and hand. A skin image refers to an image of the same area of a person captured at successive timings, and made up of, for example, a moving image or a plurality of still images.

The image acquirer 101 may either acquire a skin image by capturing an image, or acquire a skin image by acquiring the data of a skin image captured by, for example, another apparatus. When adapted to capture an image, the image acquirer 101 is implemented by, for example, a camera including an image sensor such as a charge coupled device (CCD) or a complementary metal oxide semiconductor image sensor (CMOS).

### Pulse Wave Acquirer 102

The pulse wave acquirer 102 acquires the pre-undressing timing of pulse wave for the second time period mentioned above and the post-undressing timing of pulse wave for the third time period mentioned above. Specifically, the pulse wave acquirer 102 acquires the timing of pulse wave (to be also referred to as “pulse wave timing” hereinafter) based on time variation of luminance over a plurality of skin images. To obtain this pulse wave timing, the pulse wave acquirer 102 first acquires each of the skin images from the image acquirer 101, in association with the time point at which the corresponding skin image is captured.

For example, the pulse wave acquirer 102 identifies the location where the greatest luminance change takes place in a plurality of skin images, and uses the time waveform of luminance at the identified location to calculate pulse wave timing. Alternatively, the pulse wave acquirer 102 uses a face or hand pattern stored in advance to locate the face or hand in a plurality of skin images, and uses the time waveform of luminance at the identified location to calculate pulse wave timing. Pulse wave timing is acquired through the above calculation. Pulse wave timing refers to the time point at a predetermined location in the time waveform of luminance, that is, the time waveform of pulse wave. An example of such a predetermined location is the location of a peak in the time waveform of luminance. For example, known local search methods, including those using hill-climbing, autocorrelation, and differential function can be used to enable identification of the location of a peak in a time waveform (that is, peak search). An example of a specific hardware implementation of the pulse wave acquirer 102 is a CPU.

Generally, as the heart contracts, blood is pumped out of the heart to reach body parts such as the face or hand. The luminance of the face or hand area in a captured image varies depending on the amount of hemoglobin or other component in blood. That is, information about movement of blood can be acquired by using the time variation of the luminance of the face or hand area in the image. The pulse wave acquirer 102 acquires pulse wave timing as such information.

Pulse wave timing may be acquired by using the luminance in the green wavelength range of the skin image. This is because in an image captured by using light including the wavelength range of visible light, large changes are observed in the information representing a range of frequencies near green. In an image including a plurality of pixels, the luminance in the green wavelength range of pixels corresponding to the face or hand receiving a large flow of blood is lower than the luminance in the green wavelength range of pixels corresponding to the face or hand receiving a small flow of blood.

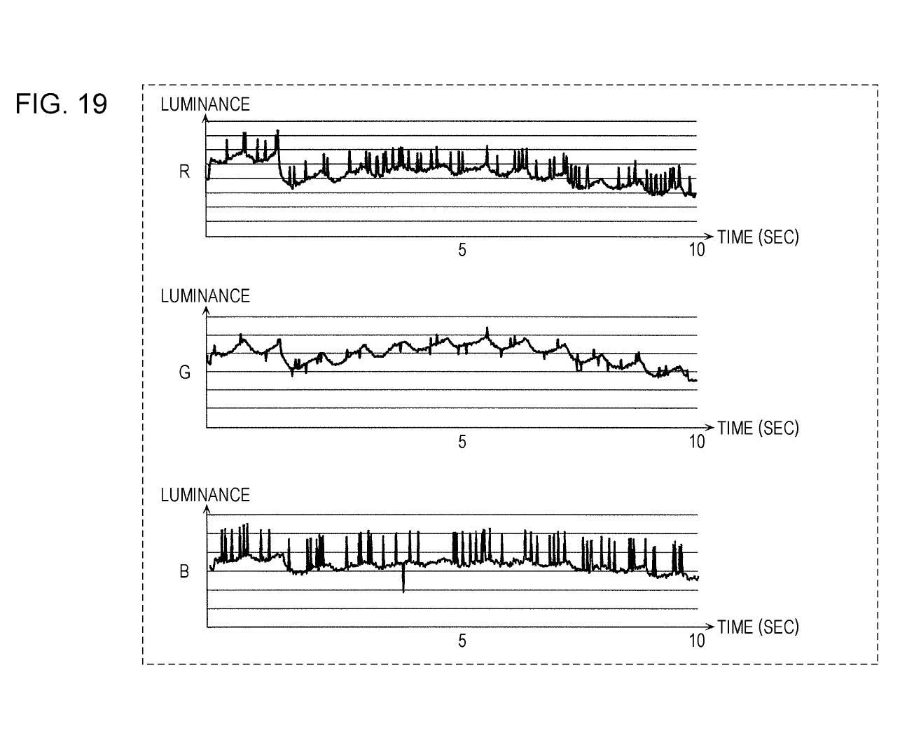

FIG. 19 illustrates an example of changes in the luminance of a skin image according to Embodiment 1. Specifically, FIG. 19 illustrates changes in the luminance of each of red (R), green (G), and blue (B) components of the cheek area in a skin image obtained through image capture by the image acquirer 101. In FIG. 19, the horizontal axis represents time, and the vertical axis represents luminance. The luminance variation illustrated in FIG. 19 indicates that the luminance of each of R, G, and B components change periodically owing to pulse wave.

When an image of the skin is captured under everyday environments, the resulting skin image contains noise owing to light scattered from the lighting or various other factors. Accordingly, signal processing may be applied to the captured image such as by using a filter to obtain a skin image containing signal changes due to pulse waves. An example of such a filter is a low pass filter. Embodiment 1 uses low-pass-filtered luminance variation of the G component.

FIG. 20 illustrates an example of calculation of pulse wave timing according to Embodiment 1. In FIG. 20, the vertical axis represents luminance, and the horizontal axis represents time. In the time waveform illustrated in FIG. 20, each of the points at times t1 to t5 is an inflection point or crest. Points on a time waveform include inflection points and peaks (including crests and troughs). Of various points included in a time waveform, each point of time at which luminance is greater (crest) or less (trough) than the luminances at both the immediately preceding time point and the immediately following time point represents pulse wave timing.

A method of identifying the location of a crest, that is, a peak search method will be described with reference to the time waveform illustrated in FIG. 20. For the time waveform of luminance illustrated in FIG. 20, the point of time t2 is taken as the current reference point. The point of time t2 and the immediately preceding point of time t1 are compared, and the point of time t2 and the immediately following point of time t3 are compared. A positive determination is made if the luminance at the reference point is greater than the luminances at both the immediately preceding point of time and the immediately following point of time. That is, it is determined that the reference point is a peak (crest), and the time point corresponding to the reference point represents pulse wave timing. A negative determination is made if the luminance at the reference point is less than at least one of the luminances at the immediately preceding point of time and the immediately following point of time. That is, it is determined that the reference point is not a peak (crest), and the time point corresponding to the reference point does not represent pulse wave timing.

Referring to FIG. 20, although the luminance at the point of time t2 is greater than the luminance at the point of time t1, the luminance at the point of time t2 is less than the luminance at the point of time t3, and hence a negative determination is made. Next, the reference point is incremented by one, with the point of time t3 now becoming the reference point. The luminance at the point of time t3 is greater than the luminances at both the point of time t2 immediately preceding the point of time t3 and the point of time t4 immediately following the point of time t3, and thus a positive determination is made. The pulse wave acquirer 102 acquires, as pulse wave timing, the point of time for which a positive determination has been made.

The pulse wave acquirer 102 acquires the pulse wave timing mentioned above before and after undressing. That is, the pulse wave acquirer 102 acquires, as the pre-undressing pulse wave timing, each time point corresponding to a peak of the time waveform in the second time period of the signal representing time variation of the luminance of the user's skin in a skin image. Further, the pulse wave acquirer 102 acquires, as the post-undressing pulse wave timing, each time point corresponding to a peak of the time waveform in the third time period of the signal representing time variation of the luminance of the user's skin in a skin image.

Acquisition of pulse wave timing may be performed by taking into the account the fact that the peak-to-peak interval ranges between, for example, 333 ms and 1000 ms, based on common knowledge about pulse wave (ranging from, for example, 60 bpm to 180 bpm). This eliminates the need to perform the above luminance comparisons for all points. Instead, luminance comparisons performed for some points are sufficient to obtain appropriate pulse wave timing. That is, the above luminance comparison may be performed by using, as a reference point, each of points that lie within the range of 333 ms to 1000 ms from the last acquired pulse wave timing. In this case, the next pulse wave timing can be acquired without performing luminance comparisons that use points preceding this range as reference points. This enables acquisition of pulse wave timing that is robust against everyday environments.

### Blood Pressure Calculator 105

The blood pressure calculator 105 uses the time difference between pre-undressing heartbeat timing and pre-undressing pulse wave timing to calculate the pre-undressing blood pressure of the user. Further, the blood pressure calculator 105 uses the time difference between post-undressing heartbeat timing and post-undressing pulse wave timing to calculate the post-undressing blood pressure of the user.

That is, the blood pressure calculator 105 calculates user's blood pressure based on the time difference (also referred to as “pulse transit time”) between the pulse wave timing obtained by the pulse wave acquirer 102 and the heartbeat timing obtained by the heartbeat acquirer 104. More specifically, the blood pressure calculator 105 determines a combination of the timing of a heartbeat and the timing of the first pulse wave that appears after the timing of the heartbeat. Then, the blood pressure calculator 105 uses the pulse transit time, which represents the time difference between the heartbeat timing and the pulse wave timing in the combination, to calculate the blood pressure of the user by multiplying the pulse transit time by a predetermined coefficient or adding a predetermined coefficient to the pulse transit time.

It is commonly said that the interval of time (pulse transit time) after blood starts of flow through the vessels following contraction of the heart until blood reaches body parts such as fingertip is correlated with blood pressure. Specifically, the higher the blood pressure, the shorter the pulse transit time, and the lower the blood pressure, the longer the pulse transit time. Techniques exist in related art for representing such a relationship by a predetermined approximate expression to estimate blood pressure. Examples of such a predetermined approximate expression may include a linear function, a combination of a plurality of linear functions, and a quadratic or more complex function.

In Embodiment 1, the blood pressure calculator 105 calculates blood pressure P by using, for example, P=αt+β as a predetermined approximate expression, where t is the pulse transit time, and α and β are coefficients (parameters). In Embodiment 1, for example, the coefficient α=−0.8, and the coefficient β=250.

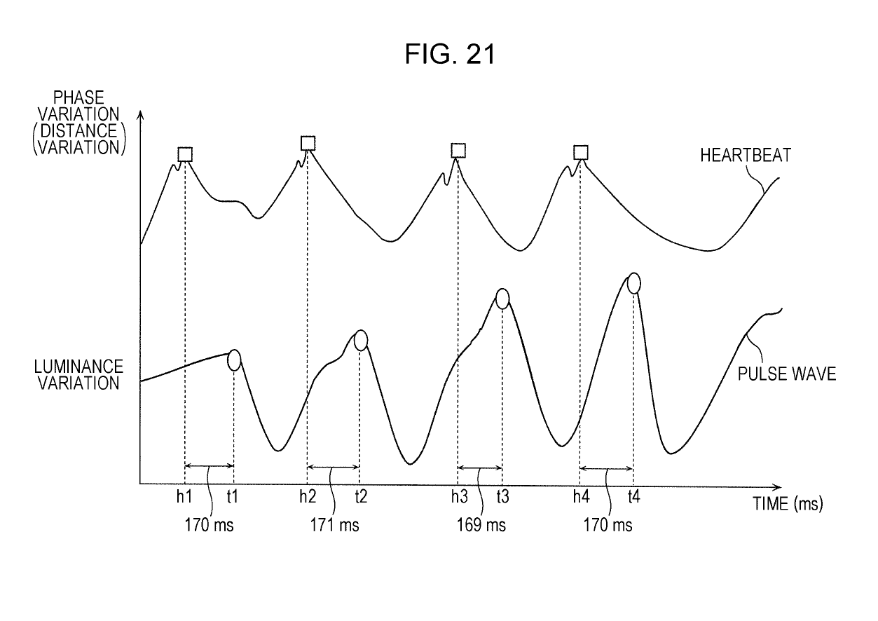

FIG. 21 illustrates calculation of blood pressure based on pulse wave timing and heartbeat timing according to Embodiment 1. In FIG. 21, the vertical axis represents changes in the phase of a millimeter wave (changes in the distance) and changes in the luminance of an image, and the horizontal axis represents time. In FIG. 21, each heartbeat timing is indicated by an unfilled square. The timings of heartbeats are represented by time points h1, h2, h3, and h4. In FIG. 21, each pulse wave timing is indicated by an unfilled circle. The timings of pulse waves are represented by time points t1, t2, t3, and t4.

A time difference exists between heartbeat timing and pulse wave timing.

The blood pressure calculator 105 calculates blood pressure based on this time difference. For example, the time difference between time point h1 and time point t1 is 170 ms. The blood pressure calculator 105 calculates blood pressure as 114 mmHg (=−0.8×170+250) by using the expression P=αt+β. The blood pressure calculator 105 may use the mean of a plurality of measurements of pulse transit time. For example, in the present case, the measurements of pulse transit time taken four times are 170 ms, 171 ms, 169 ms, and 170 ms. Accordingly, the blood pressure calculator 105 calculates the mean of those measurements as 170 ms, and hence calculates the blood pressure as 114 mmHg. Pulse wave timing or heartbeat timing often contain errors. Further, under everyday environments, the presence of noise often makes accurate measurements of these timings difficult. Therefore, using a plurality of measurements of pulse transit time allows for more robust blood pressure measurement. The blood pressure calculator 105 outputs the calculated blood pressure to the information presenter.

### Information Presenter 106

The information presenter 106 presents alert information to the user based on the difference between pre-undressing blood pressure and post-undressing blood pressure. Specifically, if the difference between the pre-undressing blood pressure and the post-undressing blood pressure is equal to or greater than a second threshold, the information presenter 106 presents the user with alert information prompting the user to refrain from immersion in water (for example, bathing).

That is, the information presenter 106 detects blood pressure variation (elevation in blood pressure) caused by undressing movement, based on the undressing condition determined by the undressing condition determiner 107 and the blood pressure calculated by the blood pressure calculator 105, and accordingly determines the information to be presented as an alert about bathing. In determining the information to be presented as an alert, the information presenter 106 determines whether or not to provide an alert (also referred to as “alert determination” or “determination of whether to permit bathing”). When the information presenter 106 determines to provide an alert, the information presenter 106 determines the information to be presented to the user as an alert.

Specifically, the information presenter 106 does not permit the user to take a bath if blood pressure variation corresponds to one of condition (a) and condition (b). Condition (a) is that the difference between the maximum and minimum values of blood pressure measured before and after each undressing movement (in the second and third time periods) during the period from the start of undressing to the completion of undressing is equal to or greater than 30 mmHg. 30 mmHg is the second threshold mentioned above. Condition (b) is that the maximum value of blood pressure measured during the period from the start of undressing to the completion of undressing is equal to or greater than 150 mmHg, and after a blood pressure equal to or greater than 150 mmHg is measured, the blood pressure remains to be equal to or greater than 150 mmHg until the completion of undressing.

Rather than measuring blood pressure at any given point (timing) in a time series, the alert information presenting apparatus 110 according to Embodiment 1 measures blood pressure while performing undressing determination in order to identify the effect of undressing activity on blood pressure that tends to show the greatest variation prior to bathing. Therefore, the information presenter 106 determines whether to provide an alert for blood pressure elevation caused by undressing.

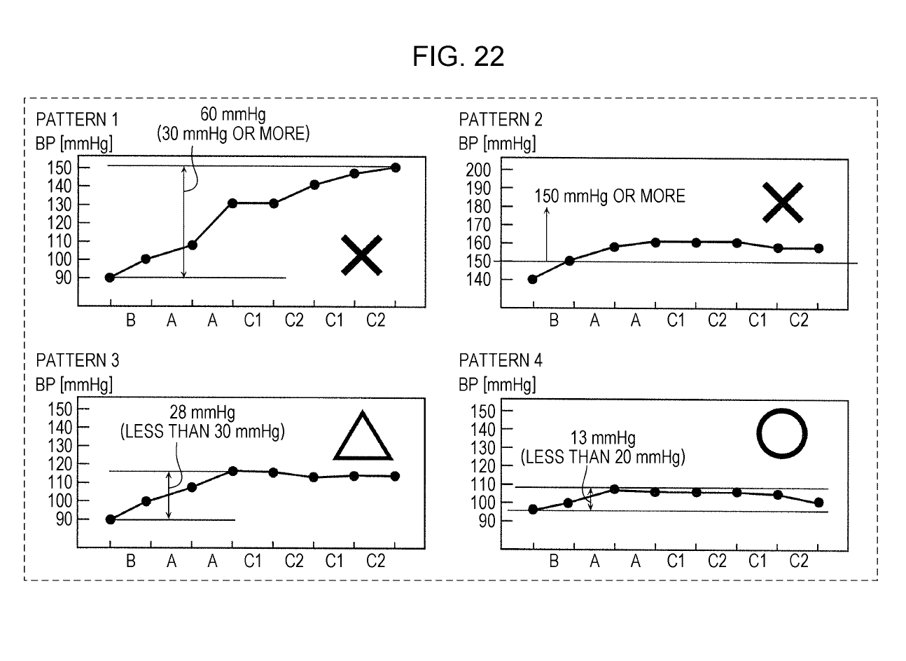

FIG. 22 illustrates an example of alert determination (determination of whether to permit bathing) performed by the information presenter 106.

In pattern 1, undressing begins with undressing movement B (for example, taking off of a shirt), with the blood pressure rising with each undressing movement performed by the user. The data on blood pressure variation during the period from the start of undressing to the completion of undressing (the blood pressures in the second and third time periods) indicates an elevation in blood pressure of 30 mmHg or more. At this time, the change in blood pressure corresponds to condition (a), and thus the information presenter 106 does not permit the user to take a bath.

In pattern 2, although large blood pressure variations are not observed (amount of blood pressure variation <30 mmHg), the blood pressure becomes equal to or greater than 150 mmHg as a result of a given undressing movement A, and that state continues until the completion of undressing. The change in blood pressure in this case corresponds to condition (b), and thus the information presenter 106 does not permit the user to take a bath. However, if the blood pressure is consistently above 150 mmHg from the start of undressing, and the amount of blood pressure variation is less than 20 mmHg, the information presenter 106 determines that the user is a hypertensive person whose blood pressure is originally high. In this case, the information presenter 106 permits bathing while alerting the user such as by advising the user to take a bath after lowering the temperature of the bath.

If the amount of blood pressure variation caused by undressing is, for example, not less than 20 mmHg but not more than 30 mmHg as in pattern 3, this change in blood pressure corresponds to neither condition (a) nor condition (b). Accordingly, the information presenter 106 permits bathing. However, since the amount of blood pressure elevation is close to 30 mmHg, the information presenter 106 may provide an alert such as by advising the user to set the temperature of the bath to a tepid temperature (38° C.). Lastly, if the amount of blood pressure variation is equal to or less than 20 mmHg, and the maximum value of blood pressure is less than 150 mmHg as in pattern 4, the change in blood pressure at that time corresponds to neither condition (a) nor condition (b). Thus, the information presenter 106 permits the user to take a bath.

Although the change in blood pressure or the maximum blood pressure during undressing is used to determine the information to be presented as an alert about bathing in the above-mentioned example, this is not to be construed in a limiting sense. For example, the alert information presenting apparatus 110 measures blood pressure regularly at times other than during bathing, and stores the mean of those blood pressure measurements (mean blood pressure) in advance. If the blood pressure has risen by 30 mmHg or more from the normal mean blood pressure, this indicates that the user's blood pressure is abnormally higher than usual, and thus the information presenter 106 may determine not to permit bathing of the user.

Although the determination of whether to provide an alert is made based on the blood pressure difference or the maximum value of blood pressure observed during the entire span of time from the start of undressing to the end of undressing, this is not to be construed in a limiting sense. As has been described above, the present disclosure makes it possible to recognize each individual undressing movement during the undressing sequence, in addition to the state immediately after the end of undressing. Further, the present disclosure enables detection of changes in blood pressure corresponding to individual undressing movements (see FIG. 15). Accordingly, the information presenter 106 may determine whether to provide an alert based on changes in blood pressure corresponding to each undressing movement.

Specifically, even if blood pressure variation corresponds to condition (a), the information presenter 106 permits bathing of the user if all of conditions (c) to (e) below are satisfied. Condition (c) is that the period during which the amount of blood pressure variation is equal to or greater than 30 mmHg (period during which the blood pressure variation follows a mountain shape) emerges in the first half of the undressing sequence from the start of undressing to its completion. Condition (d) is that after the maximum value of blood pressure is obtained, the slope of blood pressure variation remains negative until the completion of undressing. Condition (e) is that the blood pressure drops to 100 mmHg or less after the completion of undressing. The requirement that the mountain shape emerges in the first half in condition (c) means that the difference between the maximum and minimum values of blood pressure is equal to or greater than 30 mmHg, and the instant of its peak is present in the first half of the undressing sequence. In the case of finding whether the instant of peak is present in the first half or latter half of the time series, for example, movement B that involves taking off of a shirt takes a very long time, and hence the effect of such a movement on time becomes significant.

Although the method for identifying a mountain shape mentioned above involves use of the location of a peak in the undressing sequence, and the difference between the maximum and minimum values, this is not to be construed in a limiting sense. For example, the mountain shape may be statistically approximated by Gaussian fitting or other techniques to measure the instant of peak. At this time, since the exact duration of undressing differs between movements A, B, and C, the parameter σ indicating the horizontal width of the mountain shape may be adjusted for each individual undressing movement.

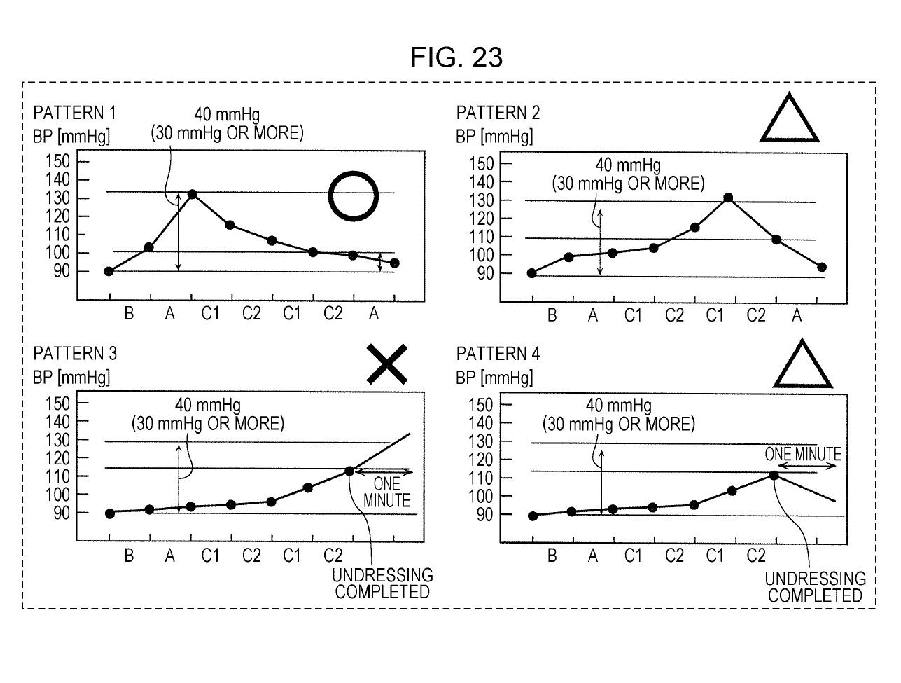

FIG. 23 illustrates another example of alert determination (determination of whether to permit bathing) performed by the information presenter 106.

In pattern 1 in FIG. 23, the blood pressure of the user is 90 mmHg at the start of undressing, which rises to 130 mmHg as the user executes undressing movement B (for example, taking off of a shirt) and undressing movement A (for example, taking off of an undershirt). Thereafter, as the user executes undressing movements C1 and C2 (for example, taking off of pants) and undressing movements C1 and C2 (for example, taking off of socks), the user gets use to the cold. This causes the user's blood pressure to drop, reaching 100 mmHg at the end of undressing. For example, starting to undress in a cold changing room such as in winter time may cause an elevation in blood pressure even in a healthy user. Thereafter, as the user gets used to the cold, the elevated blood pressure decreases in some cases. In pattern 1, although there has been a blood pressure elevation exceeding a threshold (a blood pressure elevation equal to or greater than 30 mmHg), the period of the blood pressure elevation is seen in the first half of the undressing process (undressing sequence). That is, in the case of pattern 1 with seven undressing movements included in the undressing process, a variation in blood pressure of 30 mmHg or more is reached during the first three undressing movements. Thus, condition (c) is satisfied. Further, after the blood pressure becomes maximum during the first three undressing movements, the variation of blood pressure keeps a negative slope until the completion of undressing. Thus, pattern 1 satisfies condition (d). Further, the blood pressure of the user drops to 100 mmHg or less at the completion of undressing. Thus, pattern 1 satisfies condition (e). Since pattern 1 satisfies conditions (c) to (e) as mentioned above, the information presenter 106 determines to permit bathing.

Further, in the case of pattern 1, the information presenter 106 may, for example, alert the user that a temporary elevation in blood pressure has occurred.

Although bathing is permitted if conditions (c) to (e) are satisfied in the above-mentioned example, this is not to be construed in a limiting sense. For example, the determination of whether to provide an alert may be made by factoring in whether blood pressure has actually increased as a result of a movement that is likely to elevate blood pressure. Specifically, the information presenter 106 makes reference to an undressing movement recorded in the database that is most likely to elevate the blood pressure of the user. Next, if the increase in blood pressure due to the undressing movement is less than 10 mmHg, and condition (e) is satisfied at the completion of undressing, the information presenter 106 permits bathing while presenting the user with an alert about bathing.

In pattern 2 illustrated in FIG. 23, the user's blood pressure is 90 mmHg at the start of undressing, and shows a rise of only 5 mmHg or less even after the user performs undressing movement A, which is the movement most likely to elevate the blood pressure of the user. However, the undressing movements (C2 and C1) in the latter half of the undressing process cause the blood pressure to rise to 130 mmHg. The blood pressure then decreases, reaching 95 mmHg at the completion of undressing. In this way, in pattern 2, the blood pressure rises by a total of 30 mmHg or more as a result of a plurality of undressing movements recorded in the database, including movements (for example, undressing movements C1 and C2) other than undressing movement A that is likely to elevate blood pressure. However, the elevation in blood pressure due to movement A, which is the movement most likely to elevate the blood pressure of the user, is less than 10 mmHg. Further, the blood pressure at the completion of undressing is equal to or less than 100 mmHg, and hence condition (e) is satisfied. Accordingly, the information presenter 106 permits bathing while alerting the user to the potential danger of bathing. If undressing movements C1 and C2 (for example, taking off of pants) are recorded in the database as undressing movements that are most likely to elevate blood pressure, the information presenter 106 determines not to permit bathing. This is because the peak of the mountain shape with a blood pressure variation of 30 mmHg or more occurs in the latter half of the undressing process (undressing sequence).

Further, the determination of whether to provide an alert may be made by predicting blood pressure changes that will occur after the completion of undressing. Specifically, a case is considered where, by the time when undressing is complete, the blood pressure has risen by 20 mmHg or more from the value in the second time period that occurs two timings before the completion of undressing. The second time period that occurs two timings before the completion of undressing refers to a period immediately preceding the start of an undressing movement that is performed two timings before the completion of undressing. This period will be hereinafter referred to as reference time point. In the above-mentioned case, the blood pressure is expected to rise even after the completion of undressing. Thus, the information presenter 106 does not permit bathing, and causes the blood pressure calculator 105 to measure blood pressure after the elapse of, for example, one minute. If the measurement taken after the elapse of one minute shows a total elevation in blood pressure of 30 mmHg or more, the information presenter 106 determines not to permit bathing. If the total elevation in blood pressure is less than 30 mmHg, the information presenter 106 determines to permit bathing while presenting the user with an alert about bathing.

In pattern 3, although the amount of blood pressure variation is consistently within 20 mmHg throughout the period from the start of undressing to the reference time point, the blood pressure rises by 20 mmHg or more from the reference time point to the completion of undressing. Then, the blood pressure measurement taken one minute after the completion of undressing shows a total variation in blood pressure exceeding 30 mmHg. Accordingly, the information presenter 106 determines not to permit the user to take a bath.

In the case of pattern 4, likewise, although the amount of blood pressure variation is consistently within 20 mmHg throughout the period from the start of undressing to the reference time point, the blood pressure rises by 20 mmHg or more during the period from the reference time point to the completion of undressing. However, the blood pressure decreases for one minute after the completion of undressing, resulting in a total variation in blood pressure of less than 30 mmHg. Accordingly, the information presenter 106 determines to permit bathing while alerting the user such as by advising the user to set the temperature of the bath to a tepid temperature (38° C.).

If a user previously denied of bathing as a result of bathing permission determination wishes to take a bath, the information presenter 106 may prompt the user to wait until blood pressure fluctuations settle down and the blood pressure becomes stable, and determine to permit bathing once the blood pressure stabilizes.

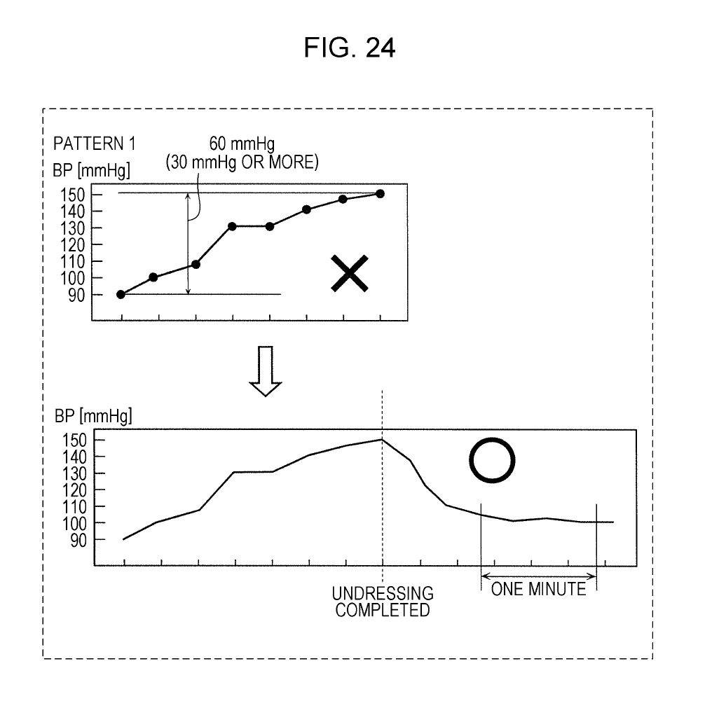

FIG. 24 illustrates a specific example of when it is determined to permit bathing, after it is once determined not to permit bathing based on pattern 1 illustrated in FIG. 22. In pattern 1, an elevation in blood pressure of 30 mmHG or more is observed during the period from the start of undressing to its completion, and thus it is once determined not to permit bathing. However, if the blood pressure decreases thereafter and remains stable for one minute, then the information presenter 106 determines to permit bathing while providing an appropriate alert. The information presenter 106 may also prompt the user to perform light exercise or instruct the user to take a deep breath in order to lower the blood pressure.

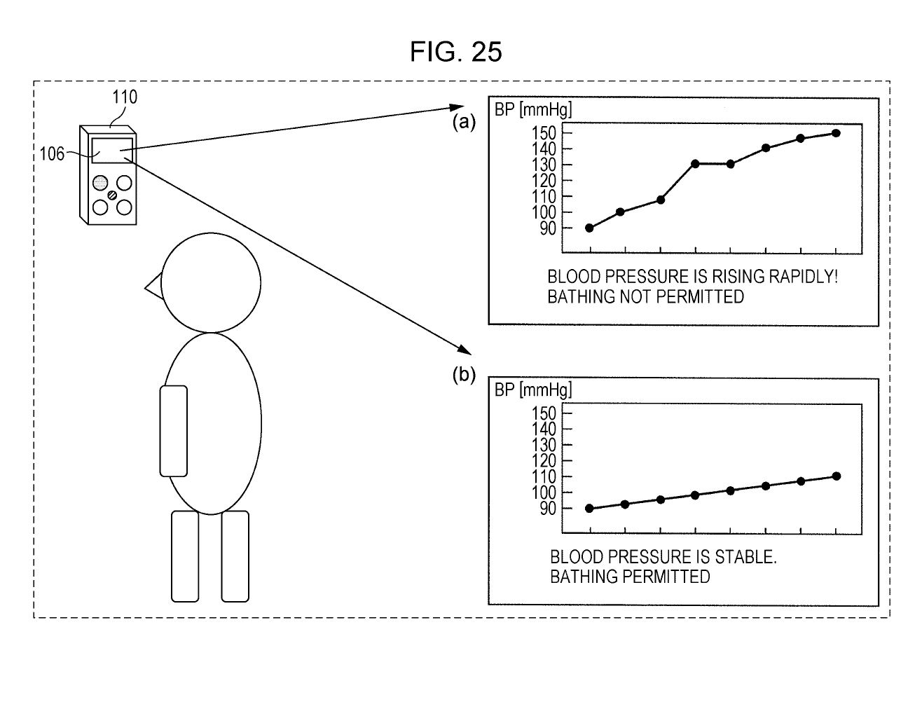

FIG. 25 illustrates an example of information presented to the user by the information presenter 106.

As illustrated in FIG. 25(a), the information presenter 106 displays variation of blood pressure measured during the undressing process. Further, the information presenter 106 also presents information indicating that the blood pressure is rising rapidly, and presents “Bathing Not Permitted” as the result of determination of whether to permit bathing. If it is determined not to permit bathing, a message prompting the user to lower the temperature of the bath may be presented to the user, in addition to information indicating that bathing is not permitted.

In another case, as illustrated in FIG. 25(b), the amount of blood pressure variation is equal to or less than 20 mmHg. Thus, the information presenter 106 presents “Bathing Permitted” as the result of determination of whether to permit bathing. Blood pressure elevation during undressing presents a serious risk factor for bathing that takes place after the undressing. Since blood pressure fluctuates constantly, a measurement taken before or after undressing may not be sufficient to find the true blood pressure elevation process, which makes it difficult to determine whether the current situation is potentially dangerous. The alert information presenting apparatus 110 according to Embodiment 1 is a contactless blood pressure measurement apparatus that measures blood pressure by using images and millimeter waves, with attention focused on the act of undressing that takes place before bathing. Rather than simply measuring blood pressure, the alert information presenting apparatus 110 determines undressing condition by using millimeter waves, and keeps track of the undressing process to enable reliable determination of blood pressure elevation caused by undressing. This makes it possible to prevent serious accidents such as cerebral infarction or myocardial infarction that occur when the user such as an elderly person takes a bath.

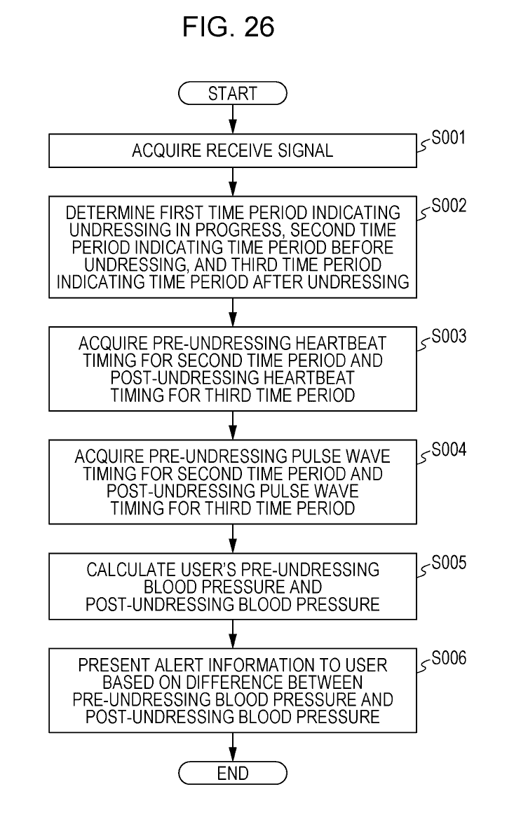

FIG. 26 is a flowchart illustrating a procedure for the processing performed by the alert information presenting apparatus 110 according to Embodiment 1.

### Step S001

The signal acquirer 103 acquires a receive signal including a radio wave reflected by the user. This radio wave is transmitted from the transmitter 103a toward the user, and is, for example, a millimeter wave.

### Step S002

The undressing condition determiner 107 calculates, by using the receive signal, the movement speed of the upper body of the user at every predetermined interval of time. The undressing condition determiner 107 determines, as the first time period, a period of time during which the receive signal shows a movement speed equal to or greater than the first threshold. The undressing condition determiner 107 determines, as the second time period, a period of time immediately preceding the first time period and during which the receive signal shows a movement speed equal to or less than the first threshold. The undressing condition determiner 107 determines, as the third time period, a period of time immediately following the first time period and during which the receive signal shows a movement speed less than the first threshold. The first time period indicates a period of time during which the user is undressing. The second time period indicates a period of time before the user undresses. The third time period indicates a period of time after the user undresses. That is, the undressing condition determiner 107 determines the first to third time periods mentioned above by using the speed of a movement performed by the user calculated by using the receive signal.

### Step S003

The heartbeat acquirer 104 uses the receive signal to acquire the pre-undressing heartbeat timing for the second time period and the post-undressing heartbeat timing for the third time period.

### Step S004

The pulse wave acquirer 102 acquires the pre-undressing pulse wave timing for the second time period and the post-undressing pulse wave timing for the third time period.

### Step S005

The blood pressure calculator 105 calculates the pre-undressing blood pressure by using the time difference between the pre-undressing heartbeat timing and the pre-undressing pulse wave timing. Further, the blood pressure calculator 105 calculates the post-undressing blood pressure by using the time difference between the post-undressing heartbeat timing and the post-undressing pulse wave timing.

### Step S006

The information presenter 106 presents alert information to the user based on the difference between the pre-undressing blood pressure and the post-undressing blood pressure.

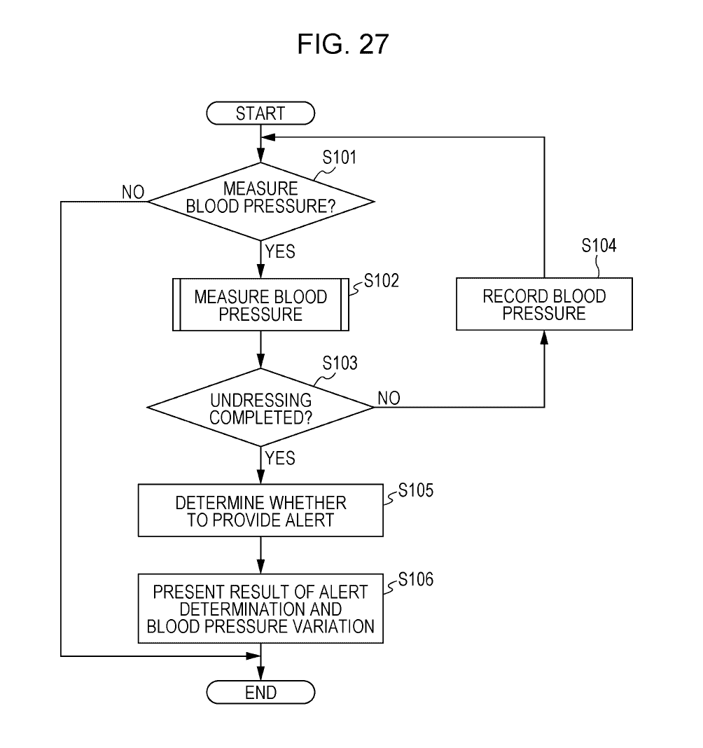

FIG. 27 is a flowchart illustrating a more specific procedure for the processing performed by the alert information presenting apparatus 110 according to Embodiment 1.

### Step S101

The undressing condition determiner 107 determines whether to measure blood pressure. If it is determined by the undressing condition determiner 107 not to measure blood pressure (step S101; NO), the alert information presenting apparatus 110 ends the processing.

### Step S102

If it is determined by the undressing condition determiner 107 to measure blood pressure (step S101; YES), the blood pressure calculator 105 measures the blood pressure of the user.

### Step S103

The undressing condition determiner 107 determines whether the user has completed undressing (undressing sequence).

### Step S104

If it is determined by the undressing condition determiner 107 that the user has not completed undressing (step S103; NO), the information presenter 106 records the blood pressure obtained by the measurement performed in step S102. Then, the undressing condition determiner 107 repeatedly executes the procedure from step S101 onward.

### Step S105

If it is determined by the undressing condition determiner 107 that the user has completed undressing (step S103; YES), the information presenter 106 determines, by using the blood pressure recorded in step S104, whether to provide an alert to the user. That is, the information presenter 106 performs the alert determination (determination of whether to permit bathing) mentioned above.

### Step S106

The information presenter 106 presents the user with the result of the alert determination performed in step S105, and blood pressure variation based on the blood pressure recorded in step S104.

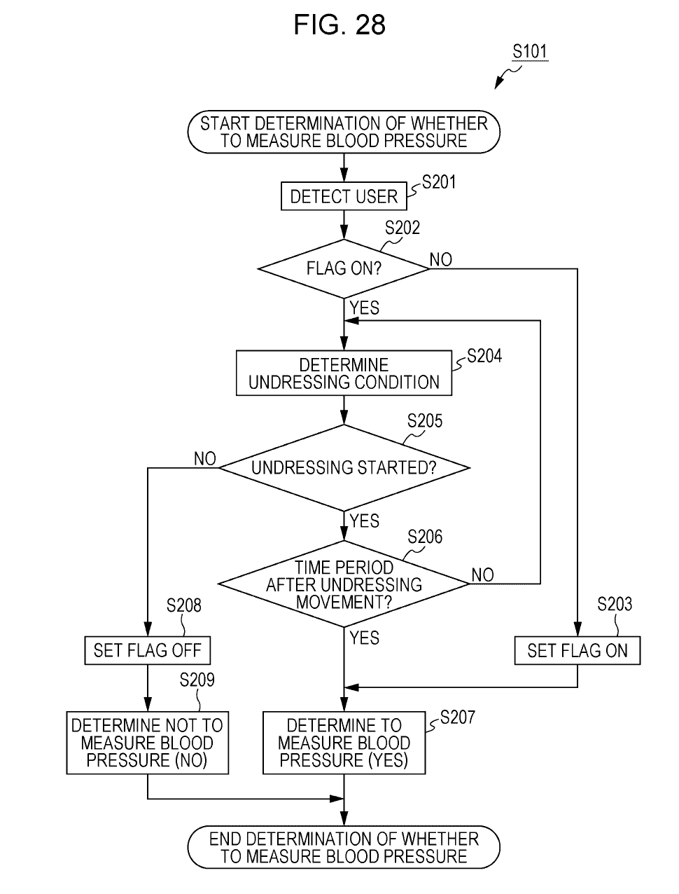

FIG. 28 is a flowchart illustrating a detailed procedure for determining whether to measure blood pressure (blood-pressure measurement determination process) executed in step S101 illustrated in FIG. 27.

### Step S201

First, the undressing condition determiner 107 detects the user by using a millimeter-wave receive signal acquired by the signal acquirer 103.

### Step S202

Next, the undressing condition determiner 107 determines whether a flag used for determining whether to measure blood pressure is ON.

### Step S203

If the flag is OFF (step S202; NO), the undressing condition determiner 107 determines that the user has entered the changing room just now, and sets the flag ON.

### Step S207

Next, after setting the flag ON in step S203, the undressing condition determiner 107 determines to measure blood pressure (step S101 in FIG. 27; YES). This means that at this time, the undressing condition determiner 107 determines the second time period indicating a period of time before the user undresses.

### Step S204

If the flag is ON (step S202; YES), the undressing condition determiner 107 determines that the user has not entered the changing room just now, and then determines the user's undressing condition.

### Step S205

At this time, the undressing condition determiner 107 determines whether the user has started undressing, based on the result of the undressing condition determination performed in step S204. That is, the undressing condition determiner 107 determines that the user has started undressing, if the user has already executed an undressing movement corresponding to movement A, B, C1, or C2 mentioned above after the flag is set ON.

### Step S206

Once the undressing condition determiner 107 determines in step S205 that the user has started undressing (step S205; YES), the undressing condition determiner 107 determines, based on the result of the determination of undressing condition in step S204, whether the current time is in a time period after the undressing movement. That is, the undressing condition determiner 107 determines whether the current time is in the third time period. If the undressing condition determiner 107 determines that the current time is not in a time period after undressing movement (step S206; NO), the undressing condition determiner 107 repeatedly executes the procedure from step S204 onward. That is, at this time, the undressing condition determiner 107 determines that the user is currently executing an undressing movement corresponding to movement A, B, C1, or C2 mentioned above. In other words, the undressing condition determiner 107 determines that the current time is in the first time period indicating that the user is undressing.

### Step S207

If the undressing condition determiner 107 determines that the current time is in a time period after undressing movement (step S206; YES), the undressing condition determiner 107 determines to measure blood pressure (step S101 in FIG. 27; YES).

### Step S208

If the undressing condition determiner 107 determines in step S205 that the user has not started undressing (step S205; NO), this means that the user has merely entered the changing room and done nothing else, and thus the undressing condition determiner 107 sets the flag OFF.

### Step S209

Next, after setting the flag OFF in step S208, the undressing condition determiner 107 determines not to measure blood pressure (step S101 in FIG. 27; NO).

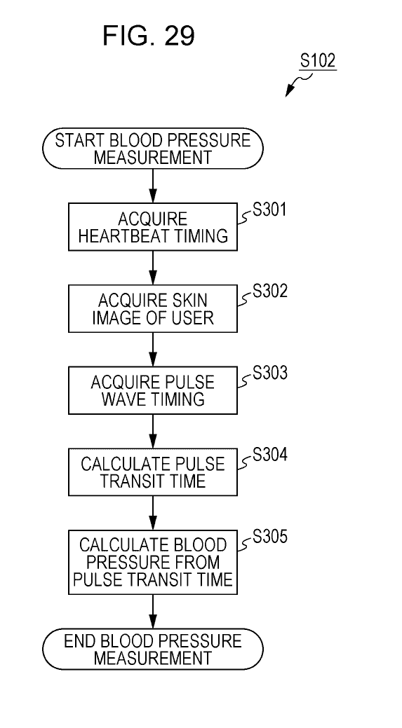

FIG. 29 is a flowchart illustrating a detailed procedure for measuring blood pressure performed in step S102 illustrated in FIG. 27.

### Step S301

The heartbeat acquirer 104 acquires the heartbeat timing of the user based on a receive signal acquired by the signal acquirer 103.

### Step S302

The image acquirer 101 acquires the skin image of the user by, for example, capturing an image of the user.

### Step S303

The pulse wave acquirer 102 acquires the pulse wave timing of the user based on the acquired skin image of the user.

### Step S304

The blood pressure calculator 105 calculates pulse transit time, which is the time difference between the acquired heartbeat timing and the acquired pulse wave timing.

### Step S305

The blood pressure calculator 105 calculates the blood pressure of the user by using the calculated pulse transit time.

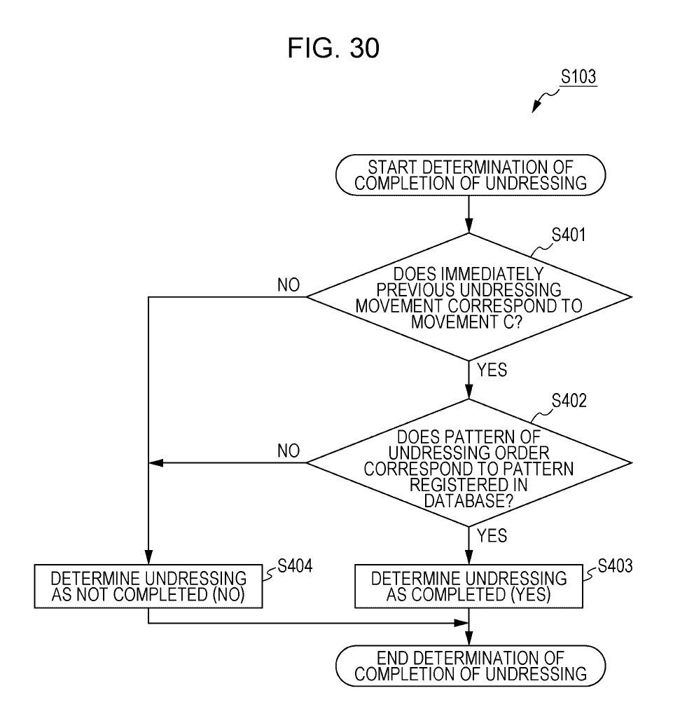

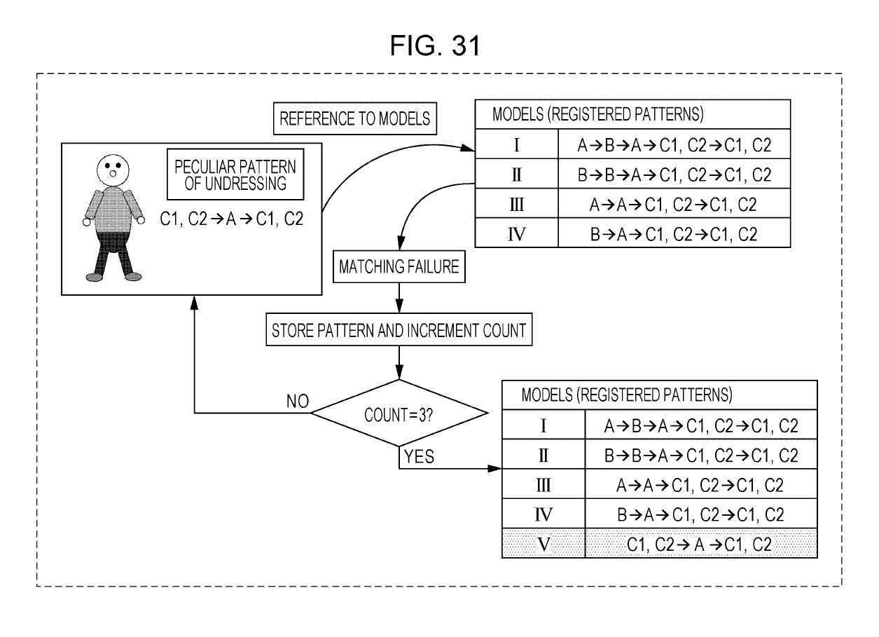

FIG. 30 is a flowchart illustrating a detailed procedure for determining whether undressing is completed (undressing-completion determination process) executed in step S103 illustrated in FIG. 27.

### Step S401

The undressing condition determiner 107 determines whether the immediately previous undressing movement determined is movement C (bringing one foot up for lower body undressing).

### Step S404

If the undressing condition determiner 107 determines in step S401 that the immediately previous movement is not movement C (step S401; NO), the undressing condition determiner 107 determines that the user has not completed undressing (step S103 in FIG. 27; NO).

### Step S402

If the undressing condition determiner 107 determines in step S401 that the immediately previous movement is movement C (step S401; YES), the undressing condition determiner 107 determines whether the pattern of the undressing order corresponds to a pattern registered in the database.

### Step S403

If the undressing condition determiner 107 determines in step S402 that the pattern corresponds to a registered pattern (step S402; YES), the undressing condition determiner 107 determines that the user has completed undressing (step S103 in FIG. 27; YES).

### Step S404

If the undressing condition determiner 107 determines in step S402 that the pattern does not correspond to a registered pattern (step S402; NO), the undressing condition determiner 107 determines that the user has not completed undressing (step S103 in FIG. 27; NO).

As described above, the alert information presenting apparatus 110 according to Embodiment 1 includes the signal acquirer 103, the undressing condition determiner 107, the heartbeat acquirer 104, the pulse wave acquirer 102, the blood pressure calculator 105, and the information presenter 106. The signal acquirer 103 acquires a receive signal including a radio wave reflected by the user. The undressing condition determiner 107 determines the first to third time periods by using the speed of a movement performed by the user calculated by using the receive signal. The first time period indicates a period of time during which the user is undressing. The second time period, which immediately precedes the first time period, indicates a period of time before the user undresses. The third time period, which immediately follows the first time period, indicates a period of time after the user undresses. The heartbeat acquirer 104 uses the receive signal to acquire the pre-undressing heartbeat timing for the second time period and the post-undressing heartbeat timing for the third time period. The pulse wave acquirer 102 acquires the pre-undressing pulse wave timing for the second time period and the post-undressing pulse wave timing for the third time period. The blood pressure calculator 105 calculates the pre-undressing blood pressure of the user by using the time difference between the pre-undressing heartbeat timing and the pre-undressing pulse wave timing. The blood pressure calculator 105 calculates the post-undressing blood pressure of the user by using the time difference between the post-undressing heartbeat timing and the post-undressing pulse wave timing. The information presenter 106 presents alert information to the user based on the difference between the pre-undressing blood pressure and the post-undressing blood pressure.

As a result, alert information is presented to the user based on the difference between the pre-undressing blood pressure and the post-undressing blood pressure, that is, the difference in blood pressure before and after undressing movement. This makes it possible to present the user with alert information based on blood pressure variation caused by the undressing movement. As a result, appropriate alert information can be presented to the user before, for example, the user takes a bath, thus keeping the user away from potentially dangerous conditions.

Further, the undressing condition determiner 107 according to Embodiment 1 determines, as the first time period, a period of time in the receive signal during which both the user's right shoulder area and the user's left shoulder area simultaneously show movement speeds equal to or higher than the first threshold.

Thus, a period of time during which the user performs movement A involving pulling clothing such as a sweater over the head, is determined as the first time period. Upon performing movement A mentioned above, the user tends to experience a significant drop in sensible temperature. Thus, movement A is more likely to elevate blood pressure than other types of undressing movements. Because alert information is presented based on the difference in blood pressure between the second time period and the third time period that immediately precede and immediately follow the first time period mentioned above, respectively, important information about potential risks can be presented to the user.

Further, the undressing condition determiner 107 according to Embodiment 1 determines, as the first time period, a period of time during which the central part of the user's upper body shows a movement speed equal to or higher than the first threshold in the receive signal.

As a result, a period of time during which the user performs movement B, such as taking off clothing, for example, a shirt by unfastening buttons on the upper central portion of the shirt, is determined as the first time period. Therefore, also when movement B mentioned above is performed in the first time period, appropriate alert information can be presented based on the difference in blood pressure between the second time period and the third time period that immediately precede and immediately follow the first time period, respectively.

Some users may undress themselves in normally inconceivable ways. Accordingly, if such a user repeatedly performs undressing in a pattern peculiar to that user, the alert information presenting apparatus 110 according to Embodiment 1 may store the pattern.

FIG. 31 illustrates an example of storing of a peculiar pattern of undressing order. According to this peculiar pattern of undressing order, the user executes the following sequence of movements: taking off of pants (movements C1 and C2) first, followed by taking off of a plurality of clothing items worn on the upper body together at once (movement A), and lastly taking off of socks (movements C1 and C2). This peculiar pattern corresponds to none of the four models (registered patterns) registered in the database. Accordingly, if undressing is performed in that peculiar pattern, the undressing condition determiner 107 temporarily stores the peculiar pattern, and counts the number of times undressing is performed in that pattern. The undressing condition determiner 107 repeats this process, and if the same pattern is repeated three times, the undressing condition determiner 107 newly adds the pattern to the existing models stored in the database.

Although Embodiment 1 assumes that the user undresses while in standing position, this is not to be construed in a limiting sense. For example, when the user undresses while sitting on a chair, the undressing condition determiner 107 determines undressing condition in sitting position mode. That is, the undressing condition determiner 107 measures the height of the user from the ground when in sitting position to calculate the difference of elevation from the user's height (body height) when in standing position. Then, the undressing condition determiner 107 deletes a block (area) corresponding to the calculated difference of elevation, and determines undressing condition.

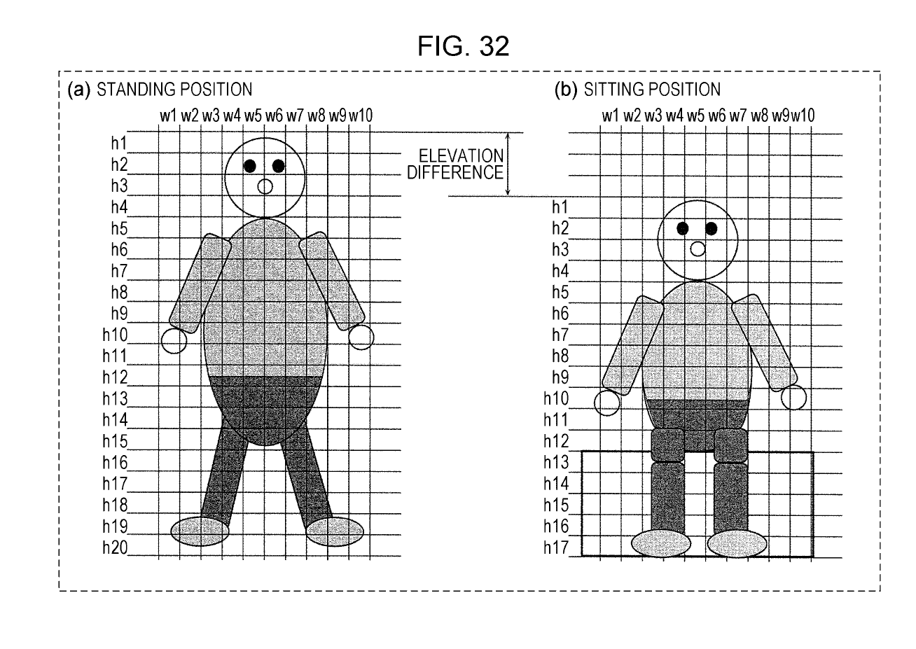

FIG. 32 illustrates determination of undressing in sitting position mode.

As the user assumes a sitting position, the undressing condition determiner 107 deletes the area (h1, w1)-(h3, w10). As a result, the area used for undressing determination is now (h4, w1)-(h20, w10). Then, the processing program of the undressing condition determiner 107 performs undressing determination with each value of h-coordinate moved down by three, that is, with the area of interest changed from (h4, w1)-(h20, w10) to (h1, w1)-(h17, w10).

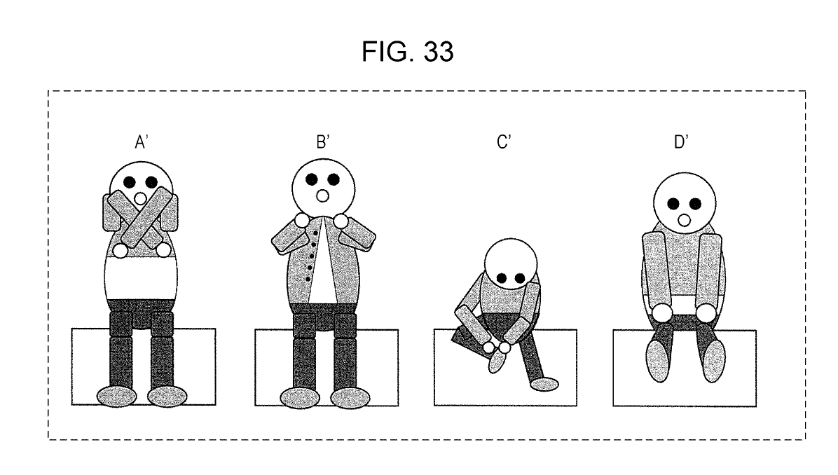

FIG. 33 illustrates an example of undressing movements performed by the user while in sitting position.

When in sitting position, the user may in some cases take off pants or a pair of socks form both feet at the same time, rather than one foot at a time. Accordingly, when a flag indicating sitting position mode is input, the undressing condition determiner 107 handles movements A′ to C′ executed in sitting position as elements used for movement determination, instead of movements A to C executed in standing position, as illustrated in FIG. 33. The undressing condition determiner 107 further adds, as an element used for undressing determination, movement D′ (both feet leaving the ground at the same time), which is not possible for the user to perform when in standing position. Thus, when performing undressing determination in sitting position mode, the undressing condition determiner 107 determines which one of movements A′ to D′ the user is performing.

Although Embodiment 1 is mainly directed to alerting the user when taking a bath, this is not to be construed in a limiting sense. For example, the alert information presenting apparatus 110 according to Embodiment 1 may be used when the user undresses before getting in a pool or before swimming. Blood pressure tends to rise during swimming, which makes it important to observe blood pressure elevations or changes while the user undresses before immersion in water. Embodiment 1 makes it possible to keep track of important changes in blood pressure associated with undressing activity also before immersion in water that is not warm but at a temperature lower than the body temperature. As a result, an appropriate alert can be provided.

### Embodiment 2

Next, Embodiment 2 of the present disclosure will be described.

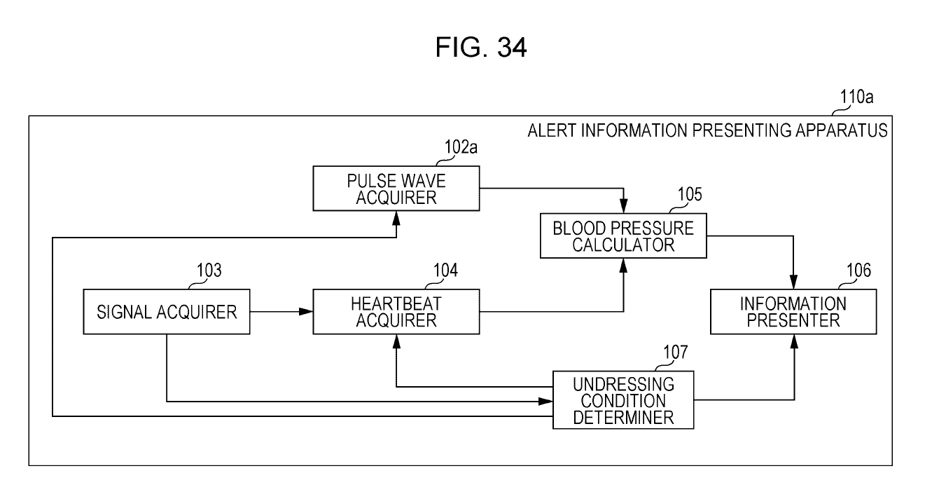

FIG. 34 is a block diagram illustrating the configuration of an alert information presenting apparatus according to Embodiment 2 of the present disclosure. An alert information presenting apparatus 110a according to Embodiment 2 includes a pulse wave acquirer 102a, the signal acquirer 103, the heartbeat acquirer 104, the blood pressure calculator 105, the undressing condition determiner 107, and the information presenter 106.

The alert information presenting apparatus 110a has basically the same configuration as that of the alert information presenting apparatus 110 according to Embodiment 1. However, in Embodiment 2, the pulse wave acquirer 102a uses a method for acquiring pulse waves different from that used by the pulse wave acquirer 102 according to Embodiment 1. Specifically, the pulse wave acquirer 102a according to Embodiment 2 includes a sole-pulse-wave sensor, and acquires the user's pulse wave timing based on an output from the sole-pulse-wave sensor.

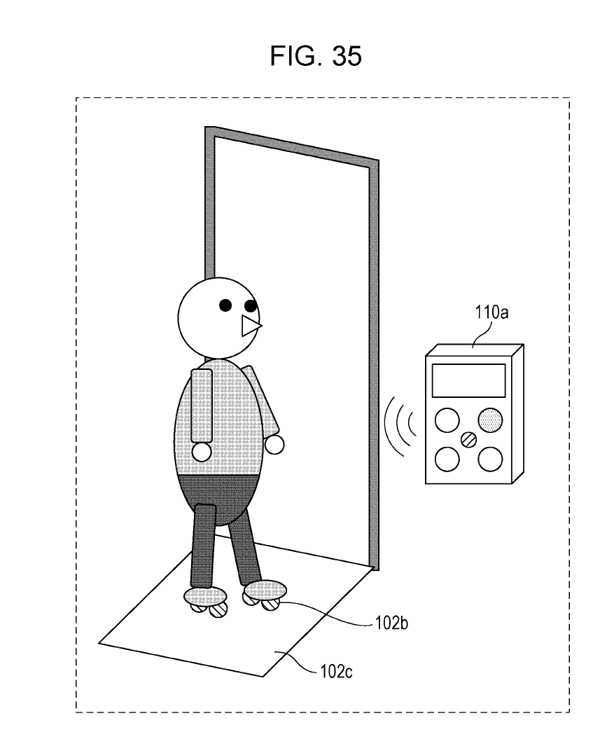

FIG. 35 illustrates how pulse wave timing and heartbeat timing are acquired by the alert information presenting apparatus 110a according to Embodiment 2.

The user steps on a sensor mat 102c attached with a sole-pulse-wave sensor 102b. The sensor mat 102c is placed in, for example, the changing room. The user usually performs undressing while on the sensor mat 102c. Thus, the user undresses while stepping on the sensor mat 102c. The alert information presenting apparatus 110a (main body) is disposed in the vicinity of the sensor mat 102c so as to acquire a receive signal representing a millimeter wave reflected by the user.

Thus, the pulse wave acquirer 102a acquires pulse wave timing based on an output from the sole-pulse-wave sensor 102b. Further, as in Embodiment 1, the heartbeat acquirer 104 acquires heartbeat timing based on a receive signal acquired by the signal acquirer 103. Then, the undressing condition determiner 107 performs undressing determination. The blood pressure calculator 105 calculates pulse transit time by using the time difference between the pulse wave timing and the heartbeat timing at a time before each undressing movement (second time period) or after each undressing movement (third time period). Then, the blood pressure calculator 105 calculates blood pressure based on the calculated pulse transit time. The information presenter 106 determines whether to permit bathing based on variation of the blood pressure.

### Pulse Wave Acquirer 102a

The pulse wave acquirer 102a is a component used to measure pulse wave timing by using the sole-pulse-wave sensor 102b. That is, the pulse wave acquirer 102a includes a pulse wave sensor that detects the user's pulse wave. The pulse wave acquirer 102a uses a signal indicative of the pulse wave detected by the pulse wave sensor to acquire pre-undressing pulse wave timing for the second time period and post-undressing pulse wave timing for the third time period.

Although the sole-pulse-wave sensor 102b is attached to the mat (sensor mat 102c) that is used during undressing in Embodiment 2, the sole-pulse-wave sensor 102b may be embedded into the floor in the changing room. The pulse wave acquirer 102a is able to obtain a pulse wave signal indicative of the pulse wave of the sole as the user's feet come into contact with the sole-pulse-wave sensor 102b.

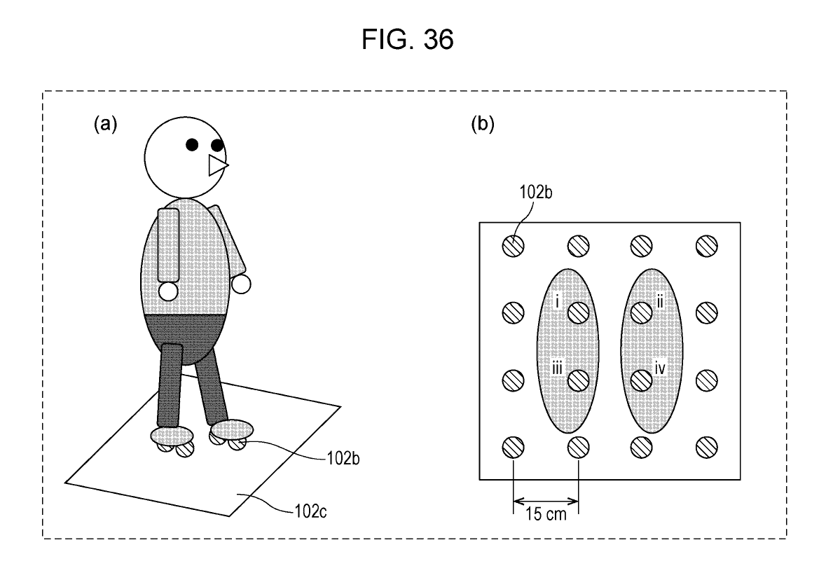

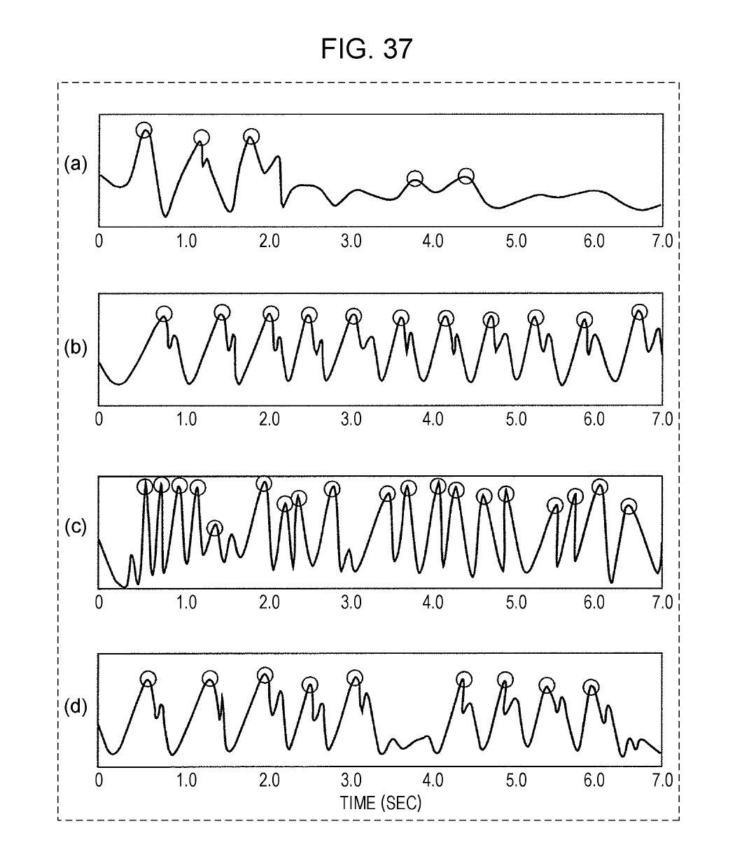

FIG. 36 illustrates an exemplary use and configuration of the sensor mat 102c. As illustrated in FIG. 36(a), as the user gets on the sensor mat 102c when undressing, the user's feet come into contact with the sole-pulse-wave sensor 102b. This allows the pulse wave acquirer 102a to acquire a pulse wave signal of the sole from the sole-pulse-wave sensor 102b, and acquire pulse wave timing based on the pulse wave signal.

When undressing, the user may not necessarily put a foot back in the same place again every time the user lifts the foot off the sensor mat 102c. Accordingly, as illustrated in FIG. 36(b), the sensor mat 102c is attached with a plurality of sole-pulse-wave sensors 102b. Each of the sole-pulse-wave sensors 102b is a combination of a photoplethysmographic sensor and a contact sensor. The contact sensor activates the photoplethysmographic sensor upon detecting contact with the body (for example, the sole) of the user. This photoplethysmographic sensor irradiates the user's body with light, and detects the user's pulse wave based on, for example, changes in the absorbance of light reflected by blood vessels or other areas inside the body. Then, the photoplethysmographic sensor outputs a pulse wave signal indicating the detected pulse wave.

The above detection can be performed with no problem as long as at least one sole-pulse-wave sensor 102b comes into contact with the user's foot when the user puts his or her foot on the sensor mat 102c. Accordingly, each of the sole-pulse-wave sensors 102b is disposed such that the distance between the sole-pulse-wave sensors 102b is, for example, equal to or less than 15 cm. This ensures that when the user puts a foot on the sensor mat 102c, one of the sole-pulse-wave sensors 102b comes into contact with the foot. That is, the contact sensor of one of the sole-pulse-wave sensors 102b comes into contact with the foot. Thus, among the plurality of sole-pulse-wave sensors 102b attached to the sensor mat 102c, the photoplethysmographic sensor of at least one sole-pulse-wave sensor 102b that is in contact with the foot activates. Accordingly, as the user puts a foot on the sensor mat 102c, at least one of the sole-pulse-wave sensor 102b comes into contact with the foot, thus allowing acquisition of the pulse wave of the user.

For example, in the case illustrated in FIG. 36(b), of 16 sole-pulse-wave sensors 102b, photoplethysmographic sensors (to be simply referred to as pulse wave sensors hereinafter) i to iv each included in the corresponding one of four sole-pulse-wave sensors 102b in contact with the foot activate. When the pulse wave acquirer 102a acquires pulse wave signals from a plurality of pulse wave sensors, the pulse wave acquirer 102a selects any pulse wave signal that satisfies the condition that the period of the pulse wave is in the range between 0.5 seconds and 1.2 seconds.

FIG. 37 illustrates an example of pulse wave signals obtained by a plurality of pulse wave sensors. The pulse wave signal illustrated in FIG. 37(a) does not satisfy the above-mentioned condition because of the disappearance of identifiable pulses at some point in the pulse wave signal. The pulse wave signal illustrated in FIG. 37(c) does not satisfy the above-mentioned condition because the (mean) pulse wave period is less than 0.5 seconds. Each of the pulse wave signals illustrated in FIG. 37(b) and FIG. 37(d) satisfies the above-mentioned condition. Accordingly, the pulse wave acquirer 102a selects the pulse wave signal with the greater number of peaks, that is, the pulse wave signal illustrated in FIG. 37(b). The pulse wave acquirer 102a acquires the pulse wave timing of the user based on the pulse wave signal selected in this way.

If the pulse wave acquirer 102a can acquire only the pulse wave signals illustrated in FIG. 37(a) and FIG. 37(c), then the pulse wave acquirer 102a selects the pulse wave signal that satisfies the above-mentioned condition for as much time as possible, like the signal illustrated in FIG. 37(a).

Although each of the sole-pulse-wave sensors 102b is placed in a fixed location in Embodiment 2, this is not to be construed in a limiting sense. For example, the sole-pulse-wave sensor 102b may, through detection of the location of a foot with a temperature sensor, automatically changes its location according to the detected location of the foot to automatically follow the foot.

Embodiment 2 allows contact of a foot with the ground to be detected by the contact sensor, simultaneously with acquisition of the pulse wave signal of the sole. Accordingly, in Embodiment 2, movement C during undressing mentioned above (undressing movement performed with one foot not touching the ground) can be detected by the pulse wave acquirer 102a in addition to the undressing condition determiner 107. For example, in some cases, the user may take off socks without hardly any movement, which makes the undressing condition determiner 107 unable to detect movement C. Even in such cases, the pulse wave acquirer 102a is able to detect whether one of the feet is in contact with the ground, that is, the pulse wave acquirer 102 is able to detect movement C. As a result, the undressing condition determiner 107 can determine whether the user has completed undressing by receiving the result of detection performed by the pulse wave acquirer 102a.

Although Embodiment 2 uses a contact sensor as a component to determine the state of contact with the ground, this is not to be construed in a limiting sense. For example, a force plate may be embedded in the sensor mat 102c or the floor in parallel with the photoplethysmographic sensor to enable determination of the balance or distribution of force exerted on the feet, thus enabling detection of contact with the ground.

Further, acquisition of a pulse wave signal from the sole requires stability for a predetermined duration of time. That is, when the sole-pulse-wave sensor 102b is successfully acquiring a pulse wave signal in a stable manner, the user's body is considered to be substantially stationary. Exploiting this characteristic allows for more accurate detection of a millimeter-wave receive signal by the signal acquirer 103. For example, the user gets on the sensor mat 102c, and starts to undress. Regardless of the timing within this undressing process, be it before an undressing movement, after an undressing movement, or during transitions between different undressing movements, once the sole comes into contact with the sole-pulse-wave sensor 102b and a stable pulse wave signal begins to be acquired, this triggers activation of the signal acquirer 103. This allows the signal acquirer 103 to acquire a receive signal with high accuracy while minimizing the influence of user's body movements.

The procedure for operation performed by the alert information presenting apparatus 110a according to Embodiment 2 is substantially the same as the procedure for operation according to Embodiment 1 illustrated in FIGS. 26 to 30, and its difference from Embodiment 1 resides in step S302 illustrated in FIG. 29. That is, the alert information presenting apparatus 110a according to Embodiment 2 performs the process of selecting a photoplethysmographic sensor (pulse wave sensor), instead of acquiring a skin image of the user.

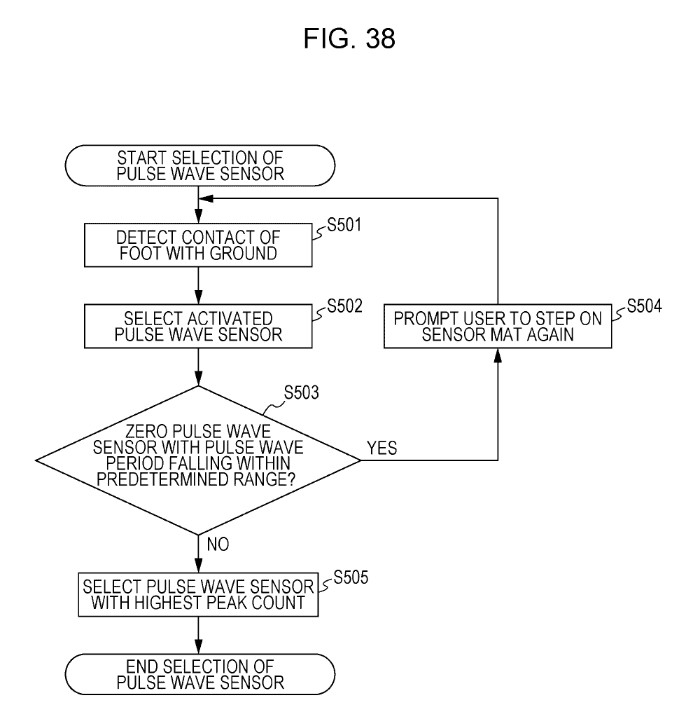

FIG. 38 is a flowchart illustrating a procedure for selecting a pulse wave sensor.

### Step S501

First, the pulse wave acquirer 102a detects that the user's foot is in contact with the ground, that is, the user's foot is in contact with the contact sensor.

### Step S502

Next, the pulse wave acquirer 102a selects, from among a plurality of pulse wave sensors attached to the sensor mat 102c (or the floor), at least one pulse wave sensor that has been activated by the contact of the user's foot with the pulse wave sensor.

### Step S503

Then, the pulse wave acquirer 102a determines whether the number of pulse wave sensors outputting pulse wave signals whose pulse wave period falls within a predetermined range (0.5 seconds to 1.2 seconds) is zero.

### Step S504

If it is determined in step S503 that the number of corresponding pulse wave sensors is zero (step S503; YES), the information presenter 106 provides an alert that prompts the user to step on the sensor mat (or the floor) again. Then, the pulse wave acquirer 102a repeatedly executes the procedure from step S501 onward.

### Step S505

If it is determined in step S503 that the number of corresponding pulse wave sensors is one or more (step S503; NO), the pulse wave acquirer 102a selects, from among the one or more pulse wave sensors, a pulse wave sensor that is outputting the pulse wave signal with the greatest number of peaks.

The pulse wave acquirer 102a acquires the pulse wave timing of the user based on the pulse wave signal output from the pulse wave sensor selected in this way.

As described above, in Embodiment 2, the pulse wave signal is detected at the sole and, at the same time, the stability of the body's condition can be observed. Since this characteristic can be exploited to enable more accurate reception of millimeter waves, heartbeat timing can be acquired with improved accuracy. Further, the pulse wave signal is acquired at the sole in Embodiment 2, thus providing improved protection of user's privacy in comparison to acquiring the pulse wave signal by capturing an image of the user's face with a camera.

Although the present disclosure uses blood pressure information in providing an alert, this is not to be construed in a limiting sense. For example, the determination of whether to provide an alert may be made by using pulse transit time calculated by using the pulse wave timing acquired by the pulse wave acquirer 102 and the heartbeat timing acquired by the heartbeat acquirer 104. If blood pressure is used for the determination, blood pressure is often measured with a cuff-type device. However, the present disclosure involves measurement of blood pressure during undressing as well as before and after undressing. Thus, use of a cuff-type device may interfere with the user's undressing action. Further, use of blood pressure estimated from pulse transit time involves use of a model formula for estimating blood pressure. This approach leads to increased processing cost in comparison to direct assessment using pulse transit time. Therefore, using pulse transit time for direct assessment of the user makes it possible to quickly determine the condition of the user and provide a notification of an alert.

### Modification 1 of Embodiment 1

Next, Modification 1 of Embodiment 1 of the present disclosure will be described.

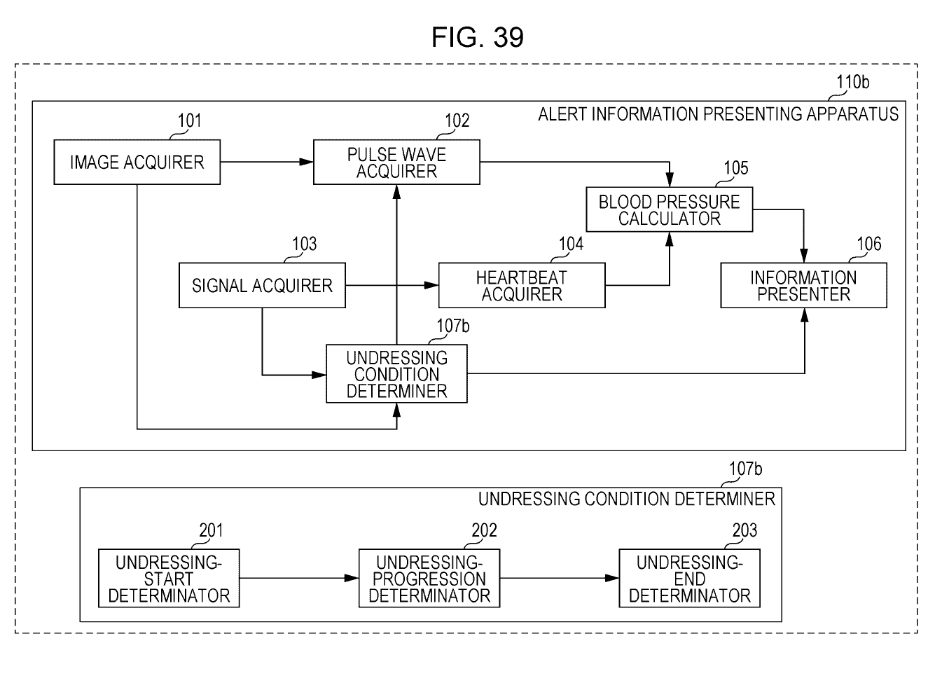

FIG. 39 is a block diagram illustrating the configuration of an alert information presenting apparatus according to Modification 1 of Embodiment 1 of the present disclosure. An alert information presenting apparatus 110b according to Modification 1 of Embodiment 1 includes the image acquirer 101, the pulse wave acquirer 102, the signal acquirer 103, the heartbeat acquirer 104, an undressing condition determiner 107b, the blood pressure calculator 105, and the information presenter 106. Further, the undressing condition determiner 107b includes an undressing-start determiner 201, an undressing-progression determiner 202, and an undressing-end determiner 203.

The undressing condition determiner 107 according to Embodiment 1 of the present disclosure includes the undressing-start determiner 201 and the undressing-end determiner 203. In Modification 1 of Embodiment 1 of the present disclosure, the undressing-progression determiner is further provided as an additional block.

The alert information presenting apparatus 110b according to Modification 1 of Embodiment 1 has basically the same configuration as that of the alert information presenting apparatus according to Embodiment 1. Further, in Modification of Embodiment 1, the undressing condition determiner 107b performs a process different from that performed by the undressing condition determiner 107 according to Embodiment 1. In particular, in Embodiment 1 of the present disclosure, the undressing-start determiner 201 and the undressing-end determiner 203 respectively determine the timing when undressing starts and the timing when undressing ends, and blood pressures before and after the undressing are measured. In Modification 1 of Embodiment 1, the state during the progression of undressing is further measured, and if the elapsed time during the course of undressing exceeds a predetermined value, the blood pressure during the progression of undressing is also measured, and depending on the measured condition, a signal is transmitted to the pulse wave acquirer to change the imaging area from a face to a hand.

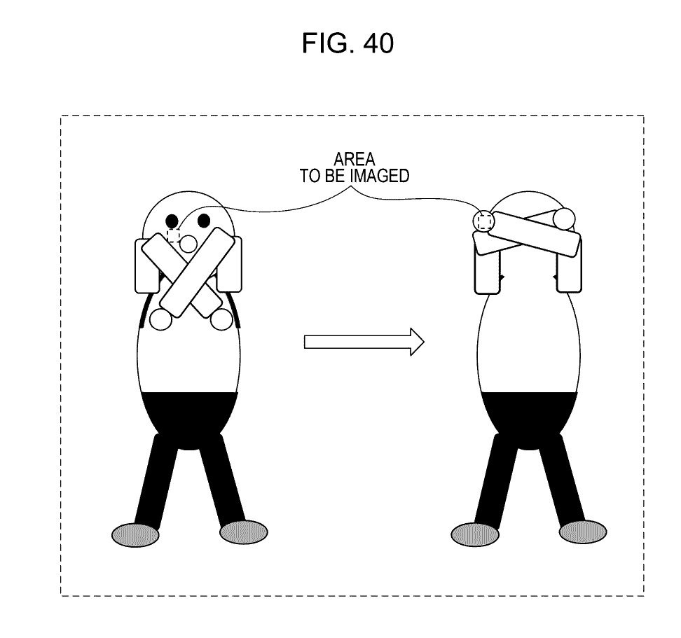

FIG. 40 illustrates how the user's undressing process is determined by the alert information presenting apparatus 110b according to Modification 1 of Embodiment 1. In Embodiment 1 of the present disclosure, user's undressing movement is determined by the undressing-start determiner 201 and the undressing-end determiner 203, blood pressure measurements are taken before and after the undressing, and an alert is provided to the user based on the difference between the measurements. However, if the user stops his or her movement in the middle of undressing, this may suggest a possibility that the user has already felt a change in blood pressure and thus the user's blood pressure has already changed. If the blood pressure has already been elevated at that point, the user may be alerted accordingly to prevent a potential danger.

For example, suppose that the user executes movement A that is characterized by taking off of clothing such as a sweater or underwear on the upper body. In this case, situations can arise where the user's undressing movement is almost complete, but the clothing gets caught on the user's head, making the user unable to fully complete the undressing. For example, this condition corresponds to when the user's movement is stopped in FIGS. 14(b), 14(c), and 14(d). The state of the user at this time is substantially the same as when undressing is complete. Thus, there is a possibility that the user may feel a change in temperature, causing an increase in blood pressure. Accordingly, even when undressing is not complete, if the user does not complete undressing after the elapse of a predetermine time, for example, one minute after the start of undressing, blood pressure may be measured in the middle of undressing. At this time, the user's face is hidden behind the clothing, and thus it is not possible to acquire the pulse wave timing of the face.

Accordingly, pulse wave timing may be acquired from a hand of the user recognized by the image acquirer 101. Further, when the face is hidden behind the clothing, the hand is located at substantially the same height as the face, and thus substantially the same estimate of blood pressure can be obtained. Therefore, it is appropriate to acquire pulse wave timing from the pulse wave of the hand, instead of the pulse wave obtained from the face, and use the acquired pulse wave timing to calculate pulse transit time and blood pressure. However, the pulse transit time calculated by using pulse wave timing and heartbeat timing, and the pulse transit time calculated by using the face's pulse wave and heartbeat timing differ. Accordingly, for example, the pulse transit time threshold for determining whether to provide an alert, and parameters used in a model for estimating blood pressure from pulse transit time are adjusted to values appropriate to individual body parts.

Suppose that a user's movement is stopped in the middle of undressing with the user's hand positioned higher than the face. In this case, the blood pressure in the user's hand is lower than the blood pressure in the heart. Accordingly, a correction may be made for the pulse transit time calculated. For example, if the hand is positioned 50 cm higher than the face, 20 ms may be added to the calculated pulse transit time in comparison to when the hand and the face are positioned at the same height.

### Undressing Condition Determiner 107b

The undressing condition determiner 107b is a component used to determine the start, progression, and end of user's undressing as well as the type of undressing movement, and change the area subject to image recognition from the face to the hand depending on the condition. The undressing condition determiner 107b includes the undressing-start determiner, and the undressing-end determiner as in Embodiment 1. Further, in Modification 1 of Embodiment 1, the undressing condition determiner 107b includes the undressing-progression determiner.

The procedure for operation performed by the alert information presenting apparatus 110b according to Modification 1 of Embodiment 1 is substantially the same as the procedure for operation according to Embodiment 1 illustrated in FIGS. 26 to 30, and its difference from Embodiment 1 resides in step S206 illustrated in FIG. 28. Specifically, the alert information presenting apparatus 110b according to Modification 1 of Embodiment 1 performs a process of observing user's condition during the course of the undressing process, and further performs an additional process depending on the observed condition.

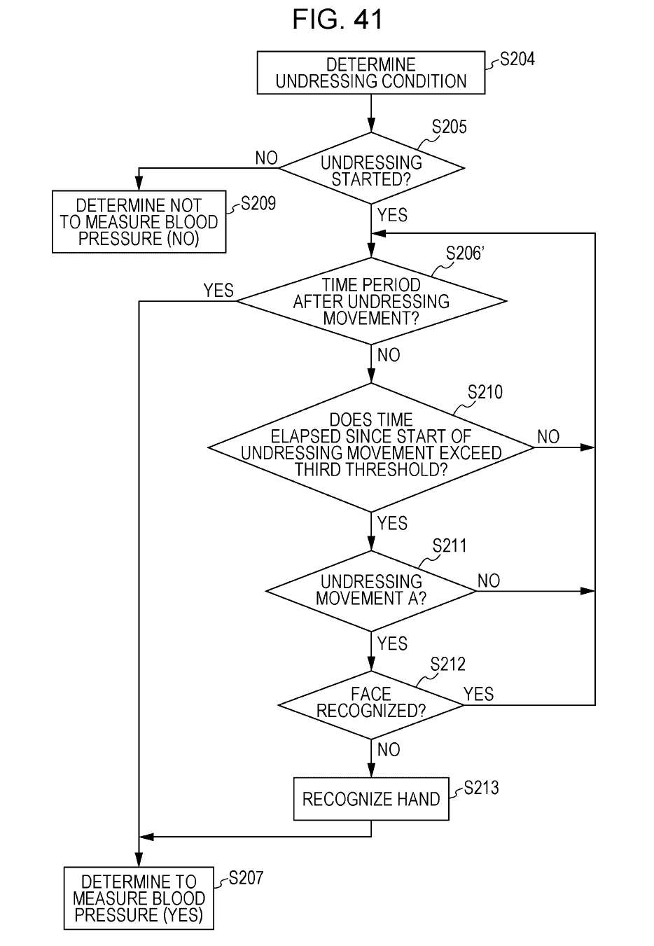

FIG. 41 is a flowchart illustrating a procedure for determining undressing condition according to Modification 1 of Embodiment 1.

First, the undressing condition is checked by the signal acquirer 103 (step S204), and it is determined whether the user has started undressing (step S205). After undressing is started, the signal acquirer 103 likewise determines whether an undressing movement has been finished, and whether it is currently in a time period between undressing movements (step S206′). If undressing is completed at this point, pulse transit time is measured by using the pulse wave acquirer 102 and the heartbeat acquirer 104, and blood pressure is calculated by the blood pressure calculator 105. If it is determined that the user has not completed undressing yet, the time elapsed after the start of undressing is measured, and it is determined whether the elapsed time exceeds a third threshold (step S210). If the elapsed time does not exceed the third threshold, the procedure returns to the step of determining whether undressing is completed, and if the elapsed time exceeds the third threshold, it is determined that the undressing movement performed by the user is movement A (step S211). If the undressing movement is not movement A, this may suggest that the user is just undressing slowly, and thus the procedure returns to step S206′. If the undressing movement performed by the user is movement A, as mentioned above, this may suggest that the user's clothing is getting caught on the head while undressing. Accordingly, whether a face can be recognized is determined next (step S212). If a face is successfully recognized, this may suggest that the user has stopped movement when movement A is still in its early stage, and thus the procedure returns to step S206′. If a face is not successfully recognized, this means that the user's movement has stopped in the middle of undressing movement A, and that the user has already felt a change in temperature (step S212). Thus, a signal is then transmitted by the undressing condition determiner 107b to the pulse wave acquirer 102 so that the user' hand is recognized (step S213), and pulse wave is acquired from the hand to estimate blood pressure from the pulse transit time between the hand and the heart (step S207).

### Modification 2 of Embodiment 1

Next, Modification 2 of Embodiment 1 will be described.

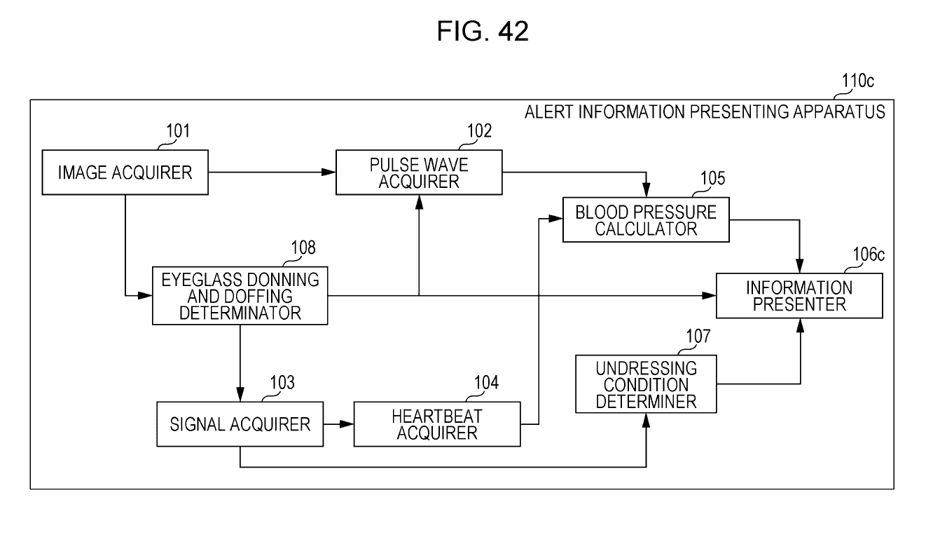

FIG. 42 is a block diagram illustrating the configuration of an alert information presenting apparatus according to Modification 2 of Embodiment 1 of the present disclosure. An alert information presenting apparatus 110c according to Modification 2 of Embodiment 1 includes the image acquirer 101, the pulse wave acquirer 102, an eyeglass donning and doffing determiner 108, the signal acquirer 103, the heartbeat acquirer 104, the undressing condition determiner 107, the blood pressure calculator 105, and an information presenter 106c. In Modification 2 of Embodiment 1, after it is recognized by the eyeglass donning and doffing determiner 108 that the user has removed eyeglasses, pulse wave timing is acquired by the pulse wave acquirer 102, and the signal acquirer 103 is activated. Then, heartbeat timing is acquired by the heartbeat acquirer 104 simultaneously with when the determination of undressing condition is started by using the undressing condition determiner 107. Then, as in Embodiment 1 of the present disclosure, the condition of undressing is measured, and changes in blood pressure before and after the undressing are calculated.

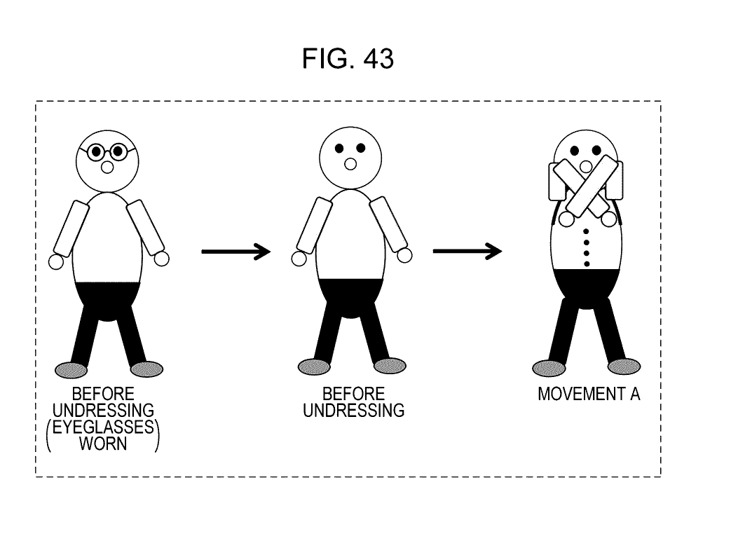

FIG. 43 illustrates an overview of Modification 2 of Embodiment 1. In Modification 2 of Embodiment 1, a characteristic behavior of a user wearing eyeglasses, namely that such a user removes eyeglasses before undressing, is exploited to adjust the timing of undressing condition measurement.

In Embodiment 1 of the present disclosure, once the user starts undressing, the condition of undressing is continuously determined throughout the period from the beginning to the end of undressing by using the signal acquirer 103. Accordingly, the alert information presenting apparatus needs to be active at all times.

In this regard, a characteristic behavior of a user wearing eyeglasses is that the user always removes the eyeglasses before undressing. Accordingly, if a user wearing eyeglasses uses the alert information presenting apparatus, the determination of undressing may be performed after removal of the eyeglasses is detected. In Modification 2 of Embodiment 1, the determination of whether the user has removed eyeglasses is made as follows. That is, a face image is acquired by the image acquirer 101, and through image recognition of the user's face, features of the eyeglasses are extracted by the eyeglass donning and doffing determiner 108 to determine the presence of eyeglasses.

In Modification 2 of Embodiment 1, the determination of whether the user is wearing eyeglasses is made through image recognition by using a face image obtained from the image acquirer 101. However, this is not to be construed in a limiting sense. For example, based on a millimeter-wave signal obtained from the signal acquirer 103, the movement of removing eyeglasses is stored as movement pattern E into the alert information presenting apparatus by the undressing condition determiner 107, and pattern matching is performed to determine whether the user is wearing eyeglasses based on the matching ratio. Image-based recognition of eyeglasses involves the user having his or her face image captured, and thus presents privacy concerns. In contrast, if data obtained by the signal acquirer is used to recognize a movement and then determine whether the user is wearing eyeglasses, the movement can be determined without capturing an image of the face.

Once the face image of the user is acquired by the image acquirer 101, and removal of eyeglasses is recognized by the eyeglass donning and doffing determiner 108, the signal acquirer 103 activates, and a determination of undressing condition begins. In this case, although the user may have already performed undressing several times prior to removing the eyeglasses, the user always takes off the eyeglasses when executing movement A as the eyeglasses get in the way during undressing activity. Meanwhile, the number of movements such as movement B and movement C remaining in the undressing sequence at that point may have decreased and, in particular, movement C representing lower body undressing may have already been finished. At this time, in Embodiment 1 of the present disclosure, the user is asked to confirm whether all undressing movements have been completed every time the user performs movement C. In this regard, in Modification 2 of Embodiment 1, if all of movements C have been completed prior to removal of eyeglasses, it is not possible to determine whether the user has completed all of undressing movements. Accordingly, in Modification 2 of Embodiment 1, the user may be asked, whenever the user's undressing condition is determined, whether the user has completed all of undressing movements. If all of movements C have been completed, movement A is more likely to occur immediately before the completion of all of undressing movements than movement B. Accordingly, in Modification 2 of Embodiment 1, the user may be asked each time movement A is finished whether the user has completed all of undressing movements.

If the user has not removed eyeglasses at the start of the first undressing movement executed, the user is likely to remove the eyeglasses prior to starting upper body undressing, particularly movement A. As described above, movement A involves taking off of, for example, a thick sweater, or taking off of underwear on the upper body which results in exposure of large areas of the skin. Thus, movement A is a movement that is most likely to cause a change in user's blood pressure.

Accordingly, in an alternative configuration, after it is confirmed that the user has removed eyeglasses, only undressing movement A is recognized, and the blood pressures before and after undressing movement A are measured to determine whether to provide an alert.

Suppose a case in which, after recognizing that the user has removed eyeglasses, the eyeglass donning and doffing determiner 108 does not recognize donning of the eyeglasses until an alert about bathing is provided based on changes in blood pressure before and after undressing. In this case, objects such as letters appear blurred to the user. Accordingly, the manner in which information is presented by the information presenter 106c may be changed. For example, for those users who have difficulty in recognizing small letters once their eyeglasses are removed, the size of letters displayed on the information presenter 106c may be increased. Further, for those users who are unable to recognize even large letters once their eyeglasses are removed, an alert may be given by sound. In particular, when at home, users are familiar with the locations of things in the place, and thus once eyeglasses are removed during undressing, many users do not put on the eyeglasses until the users finish bathing. Further, accidents such as “heat shock” tend to occur when an elderly person takes a bath alone at home. Therefore, it is extremely important to ensure that an alert be reliably presented to a user who has removed eyeglasses as in Modification 2 of Embodiment 1.

The present disclosure provides an apparatus aimed to predict and prevent heat shock accidents that may occur during bathing as well as before and after bathing. Heat shock accidents are common among the elderly. At the same time, about 80% of the elderly wear eyeglasses. Therefore, it proves useful if the alert information presenting apparatus according to the present disclosure recognizes user's eyeglasses, observes undressing condition based on whether the user has removed the eyeglasses, and alerts the user based on changes in blood pressure before and after undressing.

Although Modification 2 of Embodiment 1 is directed to users who wear eyeglasses, this is not to be construed in a limiting sense. For example, in the case of a user who wears a watch, the watch may get in the way during undressing activity, particularly when the watch becomes caught up in the clothing during movement A that involves upper body undressing. Thus, the user starts undressing after taking off the watch. Accordingly, like eyeglasses, a watch may be used as a trigger for the entire undressing action or for undressing movement A. The same applies when the user is wearing a necklace. A necklace can also become caught up in the clothing while the user executes movement A, and thus the user is likely to take off the necklace before executing movement A. Accordingly, like eyeglasses and a watch, a necklace may be used as a trigger for the entire undressing action or for undressing movement A.

### Eyeglass Donning and Doffing Determiner 108

The eyeglass donning and doffing determiner 108 is used to recognize eyeglasses from a face image of the user, and determines whether the user is wearing the eyeglasses by using data on features of the eyeglasses. After recognizing that the user has removed eyeglasses, the eyeglass donning and doffing determiner 108 instructs the signal acquirer 103 to perform undressing determination and to acquire heartbeat timing, and instructs the pulse wave acquirer 102 to acquire pulse wave timing. Further, the eyeglass donning and doffing determiner 108 transmits, to the information presenter, the donning/doffing state of the eyeglasses at the time when all of undressing movements are completed.

### Information Presenter 106c

The information presenter 106c is a component used to determine the information to be presented as an alert based on changes in user's blood pressure before and after undressing, and present the alert information to the user. The information presenter 106c performs the same process as that in Embodiment 1 of the present disclosure, and includes presentation equipment such as a display. In Modification 2 of Embodiment 1, further, the information presenter 106c changes the information to be presented, based on an instruction given from the eyeglass donning and doffing determiner 108, and includes a speaker for outputting sound.

The procedure for operation performed by the alert information presenting apparatus 110c according to Modification 2 of Embodiment 1 is substantially the same as the procedure for operation according to Embodiment 1 illustrated in FIGS. 26 to 30, and its difference from Embodiment 1 resides in steps S201 to S204 illustrated in FIG. 28. That is, the alert information presenting apparatus 110c according to Modification 2 of Embodiment 1 additionally includes a process of determining, prior to determining user's undressing condition, whether the user has removed eyeglasses, and starts to perform undressing determination after recognizing that the user has removed the eyeglasses.

Further, Modification 2 of Embodiment 1 also differs from Embodiment 1 in steps S103 to S106 in FIG. 27. That is, the alert information presenting apparatus 110c according to Modification 2 of Embodiment 1 additionally includes a process of determining whether the user is wearing eyeglasses at the time when the user's undressing activity is completed, and changes the manner in which information is presented to the user depending on whether the user is wearing the eyeglasses.

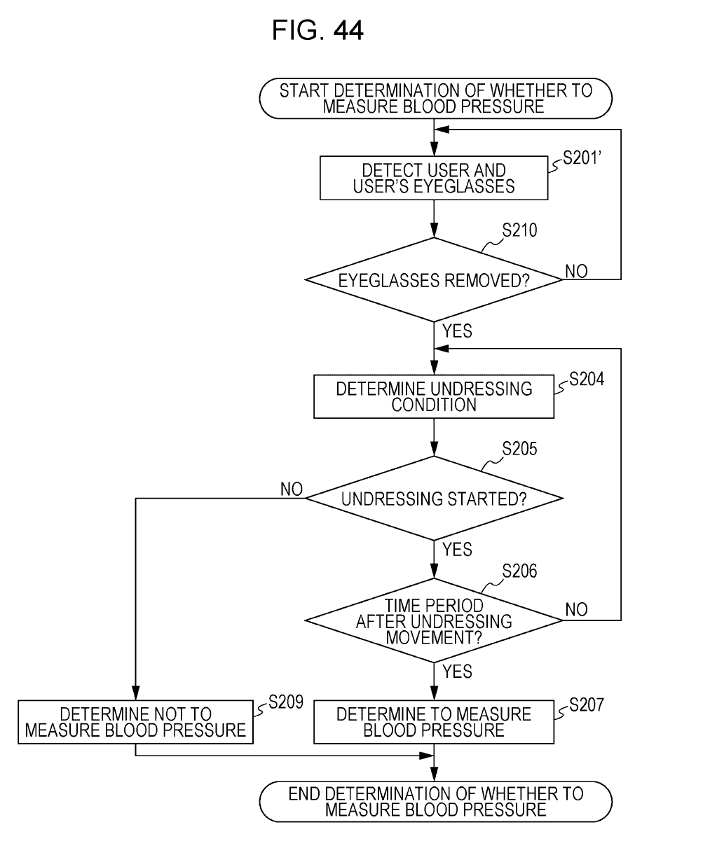

FIG. 44 is a flowchart illustrating a procedure for determining undressing condition according to Modification 2 of Embodiment 1.

First, the image acquirer 101 recognizes that the user has entered the changing room. At the same time, if the user is wearing eyeglasses, the image acquirer 101 recognizes the eyeglasses from a face image of the user (step S201′). Next, the eyeglass donning and doffing determiner 108 determines whether the user has removed the eyeglasses (step S210). If the user has not removed the eyeglasses, the eyeglass donning and doffing determiner 108 again continues to recognize the presence of the user and wearing of the eyeglasses by the user, and when the user ceases to be detected, it is determined that the user has not performed undressing in the changing room, and the procedure is ended without measuring blood pressure. If the user has removed the eyeglasses, as in Embodiment 1 of the present disclosure, undressing condition is measured, and every time undressing movement is finished, it is determined to measure blood pressure. Then, this procedure is ended. Further, if the user has removed the eyeglasses but has not started undressing movement yet, it is determined that the user is currently in a state in which the user does not perform undressing even though the eyeglasses are removed, such as when the user is standing in front of the mirror of the wash stand. Thus, it is determined not to measure blood pressure, and this procedure is ended.

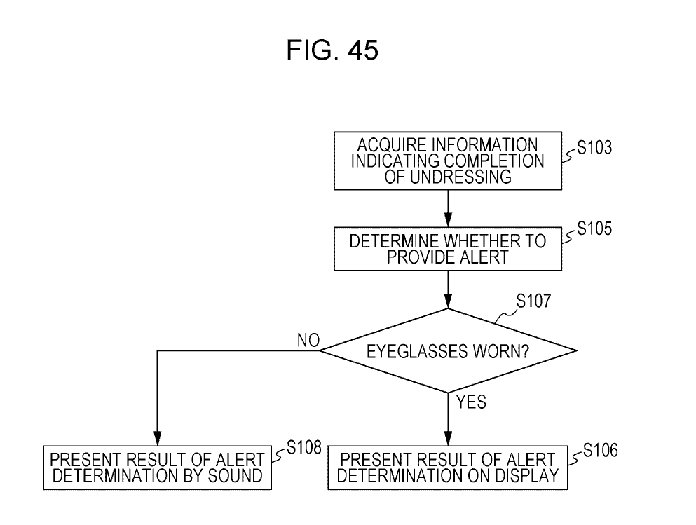

FIG. 45 is a flowchart illustrating a procedure for presenting information according to Modification 2 of Embodiment 1.

First, the undressing condition determiner 107 recognizes that the user has completed undressing (step S103). Then, the information presenter 108c determines whether to provide an alert, based on changes in blood pressure before and after undressing (step S105). Then, it is determined whether the user is wearing eyeglasses at the time when all of the user' undressing movements are completed (step S107). If the user is wearing eyeglasses at that time, the user is considered to be able to see information displayed on the display as usual, and thus the result of alert determination is presented on the display (step S106). If the user is not wearing eyeglasses even after the completion of all of undressing movements, it is likely that the user's vision is blurry and poor. Accordingly, the result of alert determination is presented to the user by, for example, sound.

Although Modification 2 of Embodiment 1 has been described as if limited to users wearing eyeglasses, this is not to be construed in a limiting sense. For example, in an alternative configuration, whether a user wears eyeglasses is recorded in the database, and at the time when the user enters the changing room, personal authentication is performed by image recognition or other methods to select a suitable process. This database may be added by the user. Alternatively, when a new user enters the changing room, the user may be asked to input data.

### Embodiment 3

Embodiment 3 of the present disclosure will be described below.

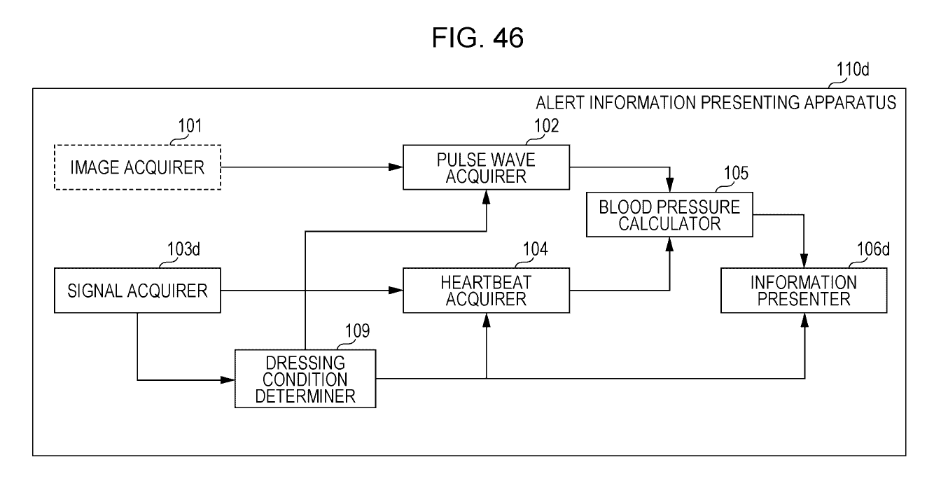

FIG. 46 a block diagram illustrating the configuration of an alert information presenting apparatus according to Embodiment 3 of the present disclosure. An alert information presenting apparatus 110d according to Embodiment 3 includes the image acquirer 101, the pulse wave acquirer 102, a signal acquirer 103d, the heartbeat acquirer 104, a dressing condition determiner 109, the blood pressure calculator 105, and an information presenter 106d. In Embodiment 3, the signal acquirer 103d detects that the user has moved from the bathing room to the changing room. Further, the dressing condition determiner 109 determines dressing condition continuously from immediately after bathing and measures changes in blood pressure before and after dressing, and further provides an instruction to periodically measure blood pressure over a period up to a first dressing event to measure changes in blood pressure up to the first dressing event following bathing as well as changes in blood pressure caused by the subsequent dressing movements. Then, the information presenter 106d alerts the user based on blood pressure information obtained from the blood pressure calculator 105 and dressing condition information obtained from the dressing condition determiner 109.

The alert information presenting apparatus 110d according to Embodiment 3 has basically the same configuration as that of the alert information presenting apparatus according to Embodiment 1. The alert information presenting apparatus 110d according to Embodiment 3 includes the dressing condition determiner 109 instead of the undressing condition determiner 107. Although the undressing condition determiner 107 according to Embodiment 1 determines undressing of the user, the dressing condition determiner 109 according to Embodiment 3 performs a process of determining dressing of the user continuously from immediately after bathing. Further, the signal acquirer 103d recognizes the instant when the user finishes bathing and leaves the changing room, and indicates to the dressing condition determiner 109 that the user has finished bathing. Then, the dressing condition determiner 109 instructs the pulse wave acquirer 102 and the heartbeat acquirer 104 to respectively measure the pulse wave timing immediately after bathing and the heartbeat timing immediately after bathing.

Next, a process for determining dressing condition will be described. As illustrated in FIG. 10, like the process for determining undressing according to Embodiment 1, the process for determining dressing condition involves measuring speed changes by using a millimeter-wave signal to determine dressing movement.

As described above, undressing involves three basic movements, movement A, movement B, and movement C as illustrated in FIG. 11, and each movement is classified by body-part-specific characteristic speed changes associated with each individual movement. The same movement determination process is used also for determination of dressing. Dressing movements corresponding to movement A, movement B, and movement C, which are undressing movements, are respectively represented by movement A′, movement B′, and movement C′. As for the process of determining undressing movement based on the time-series pattern as illustrated in FIG. 14, the order of the time-series data used for that process is reversed, and the resulting data is used for determination of dressing movement.

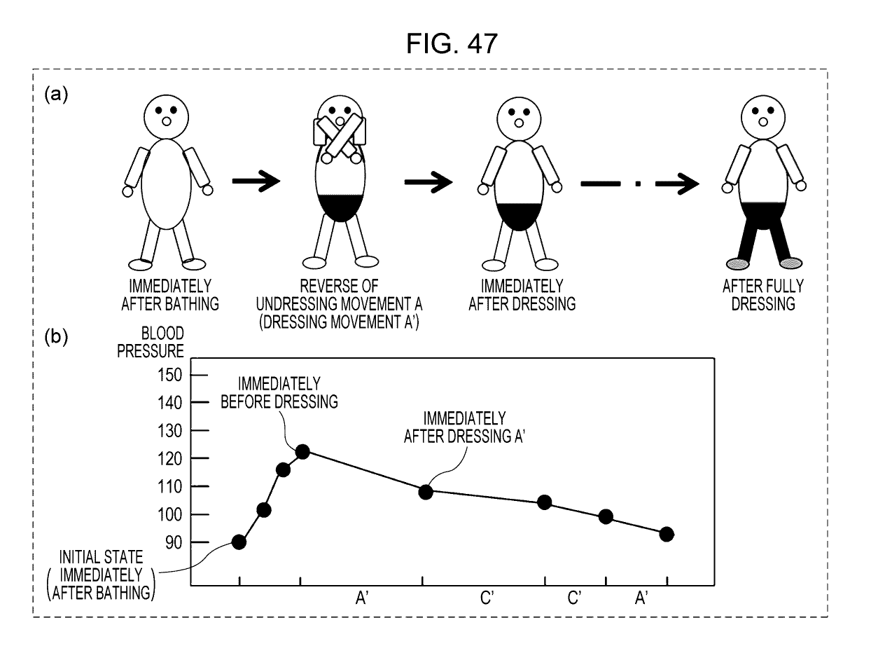

FIG. 47 illustrates how user's dressing condition after bathing is determined according to Embodiment 3. FIG. 47(a) illustrates an example of a dressing process. Immediately after bathing, the user starts to dress after drying off the body first. Since the user is naked immediately after bathing, the user's body is sensitive to temperature changes, and as the body starts to feel the cold, blood pressure increases. Accordingly, when the user dresses, unlike during undressing, it is important to measure blood pressure by using the pulse wave timing and the heartbeat timing that are obtained at the beginning of the dressing process immediately after bathing when the user is still naked. Further, if a long time elapses between the time immediately after bathing and the time when the user starts dressing, the blood pressure rises even more with the elapse of time. Accordingly, another important issue is how to reliably measure blood pressure over the period from immediately after bathing to the start of dressing. Then, as in the determination of undressing, blood pressure is measured in accordance with dressing movement, thus allowing more fine-tuned alerts to be presented to the user.

FIG. 47(b) illustrates an example of changes in user's blood pressure immediately after bathing. If the changing room is cold, upon entry into the changing room, the user feels the cold on the skin of the entire body as the user is naked, causing a rise in blood pressure. In the case illustrated in FIG. 47(b), the blood pressure rises as the user feels the cold immediately after bathing, and then the blood pressure drops with the subsequent dressing steps.

As illustrated in FIG. 47(b), immediately after bathing, the user's blood pressure tends to progressively increase if the user remains undressed. Accordingly, after entry of the user into the changing room is recognized by the signal acquirer 103d immediately after bathing, the dressing condition determiner 109 needs to periodically measure blood pressure, not only immediately after bathing and before and after dressing but also during the time up to the first dressing event, and alert the user based on changes in the measured blood pressure. In Embodiment 3, the first dressing event refers to when the user performs movement A′ for the first time. After taking a bath, the user first puts on underwear in the sequence of dressing. This still leaves large areas of the skin exposed, and thus this state of the user can be hardly regarded as dressed. Then, to prevent the body from getting cold, the user executes movement A′ to perform upper body dressing. That is, movement A′ related to upper body dressing that has a significant effect on changes in blood pressure is regarded as the first dressing event.

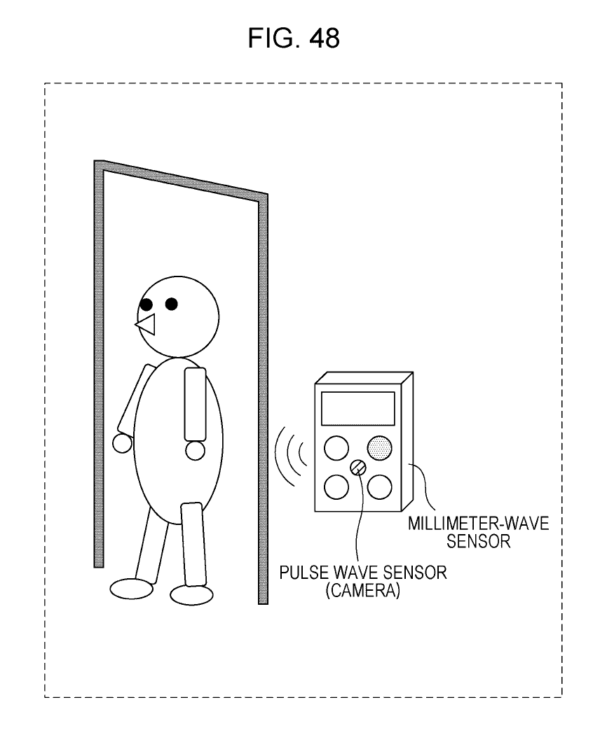

Specifically, as illustrated in FIG. 48, the signal acquirer 103d acquires a signal indicating entry of the user into the changing room immediately after bathing, and transmits the signal to the dressing condition determiner 109. Further, information about the time of entry into the changing room is simultaneously transmitted as a first time. Then, the instant the dressing condition determiner 109 obtains the signal from the signal acquirer 103d, the dressing condition determiner 109 sends instructions to the pulse wave acquirer 102 and the heartbeat acquirer 104 to measure pulse wave timing and heartbeat timing, respectively, to calculate blood pressure. After obtaining the first signal from the signal acquirer 103d, the dressing condition determiner 109 determines dressing condition by using the signal indicative of user's movement acquired from the signal acquirer 103d. Simultaneously with this determination, the dressing condition determiner 109 also measures the time elapsed. Then, every time a fourth time period, for example, 30 seconds elapses after the first time, the dressing condition determiner 109 determines that blood pressure is to be measured.

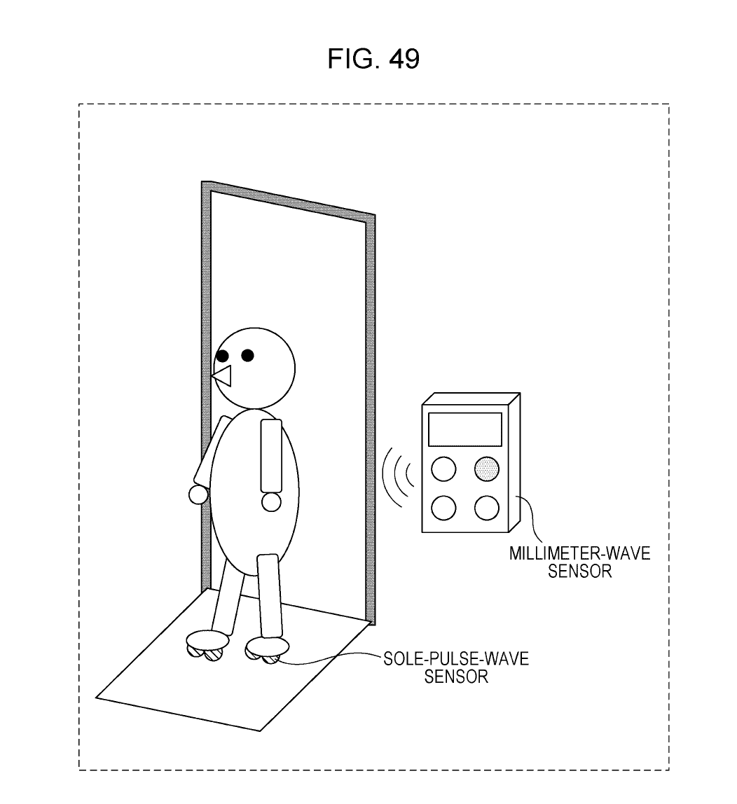

In Embodiment 3, a millimeter-wave signal acquired by the signal acquirer 103d is used to recognize the user's entry into the changing room immediately after bathing. However, this is not to be construed in a limiting sense. For example, millimeter-wave signals are absorbed by moisture. This means that in the state when the user's body has not been dried off immediately after bathing, often only a weak signal can be acquired. Accordingly, in Embodiment 3, only an image of the user's feet may be captured with the camera of the image acquirer 101 to determine whether the user has entered the changing room while protecting the user's privacy. Alternatively, as illustrated in FIG. 49, the user may be detected with, for example, a sensor embedded in a drainage mat. After taking a bath, to drain off the water from the feet, the user often steps on a mat when entering the changing room and drains off the water from the sole. Accordingly, for example, a human sensor or pulse wave sensor inside the mat is used in conjunction with this action to enable detection of the user while protecting the user's privacy.

Next, an alerting process will be described.

In Embodiment 1, undressing condition is measured, and an alert is given based on changes in blood pressure caused by undressing. In Embodiment 1, until the user starts undressing, the user is unlikely to feel temperature changes and hence blood pressure is unlikely to change, and thus there is no particular need to measure blood pressure. In contrast, in Embodiment 3, after entering the changing room again after bathing, the user at this time is more sensitive to the variation in temperature between the bathing room and the changing room until the user starts dressing. This makes it necessary to continuously measure blood pressure during the period from when the user enters the changing room to when the user performs dressing. Examples of an alert to be given about changes in blood pressure at this time include an instruction to prompt the user to dress quickly.

Further, although alert information is presented mainly to the user himself or herself in Embodiment 1, this may not necessarily be the case in Embodiment 3. In Embodiment 1, based on changes in blood pressure occurring while the user undresses before taking a bath, an alert is given to the user himself or herself to prevent potential accidents due to a sudden drop in blood pressure that is unlikely to occur during the subsequent bathing. However, one major aim of Embodiment 3 is to minimize rapid increases in blood pressure that occur in the changing room in the user who has already finished bathing. Accordingly, although an alert is given also to the user himself or herself, cases may also arise where the user experiences an acute rise in blood pressure the instant the user enters the changing room, resulting in fainting of the user. At this time, to treat or rescue the user as quickly as possible, it is important to present information not to the user himself or herself but to, for example, a third person such a family member.

Accordingly, a characteristic feature of the information presenter according to Embodiment 3 resides in that the information presenter presents information not only to the user himself or herself but also to a third person who is present near the user.

Further, a characteristic feature of Embodiment 3 resides in that the information to be presented as an alert is determined by using not only changes in blood pressure after bathing but by also using changes in blood pressure before bathing. The extent of blood pressure drop during bathing can be roughly determined by using the extent of blood pressure elevation due to changes in blood pressure during undressing, and the blood pressure at the time when the user enters the changing room after bathing. By observing subsequent increases in blood pressure, more fine-tuned alerts can be provided.

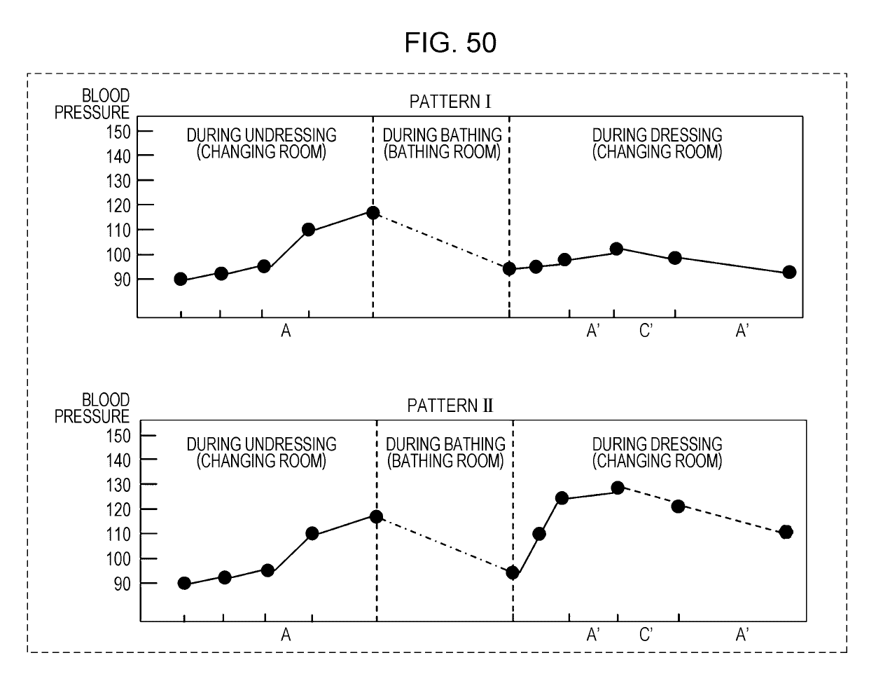

FIGS. 50, 51, and 52 illustrate examples of patterns of changes in user's blood pressure after the user enters the changing room after bathing.

First, pattern I illustrated in FIG. 50 shows a rise in blood pressure of 25 mmHg prior to bathing, from 90 mmHg to 115 mmHg. However, the blood pressure at the time when the user enters the changing room again is 20 mmHg lower than the blood pressure immediately before entry into the bathing room. The subsequent rise in blood pressure is not so significant, with the blood pressure changing by not more than 30 mmHg up to the beginning of the first dressing event. Thereafter, the blood pressure gradually drops as the user puts on more items of clothing.

That is, if the blood pressure immediately after entry into the changing room is below the blood pressure immediately before entry into the bathing room, and the increase in blood pressure over a period from the re-entry into the changing room after bathing to the first dressing event is less than a fourth threshold of, for example, 30 mmHg, the information presenter 106d determines this pattern of user's blood pressure variation as pattern I.

At this time, the user has been sufficiently warmed up by bathing, and is able to maintain the body temperature even after bathing. Thus, the information presenter 106d notifies the user that the current situation is safe by constantly providing an indication to that effect on the display. Although the information presenter 106d provides a notification that the current situation is safe on a display in the case of pattern I, this is not to be construed in a limiting sense. If the current situation is safe, there is no need to make the user aware of the alert information presenting apparatus, and thus no particular instruction may need to be given.

Next, in pattern II illustrated in FIG. 50, the changes in blood pressure follow the same pattern as pattern I mentioned above until the user enters the changing room after taking a bath. However, an elevation in blood pressure of 30 mmHg or more occurs during the period from immediately after the entry into the changing room to the first dressing event.

That is, if the blood pressure immediately after entry into the changing room is below the blood pressure immediately before entry into the bathing room, and the increase in blood pressure from when the user enters the changing room again after bathing to when the first dressing event takes place is equal to or greater than the fourth threshold of, for example, 30 mmHg, the information presenter 106d determines this pattern of user's blood pressure variation as pattern II.

At this time, measurement of blood pressure taken during the time up to the first dressing event shows a significant rapid rise in blood pressure. Such a rapid change of blood pressure puts the user at risk for a potentially very dangerous condition. Accordingly, the information presenter 106d presents information to that effect to the user and a third person. At this time, a message “Please dress quickly.” is presented on the display. For the third person, for example, information indicating that the user is in a potentially dangerous condition may be presented to the mobile terminal of the third person.

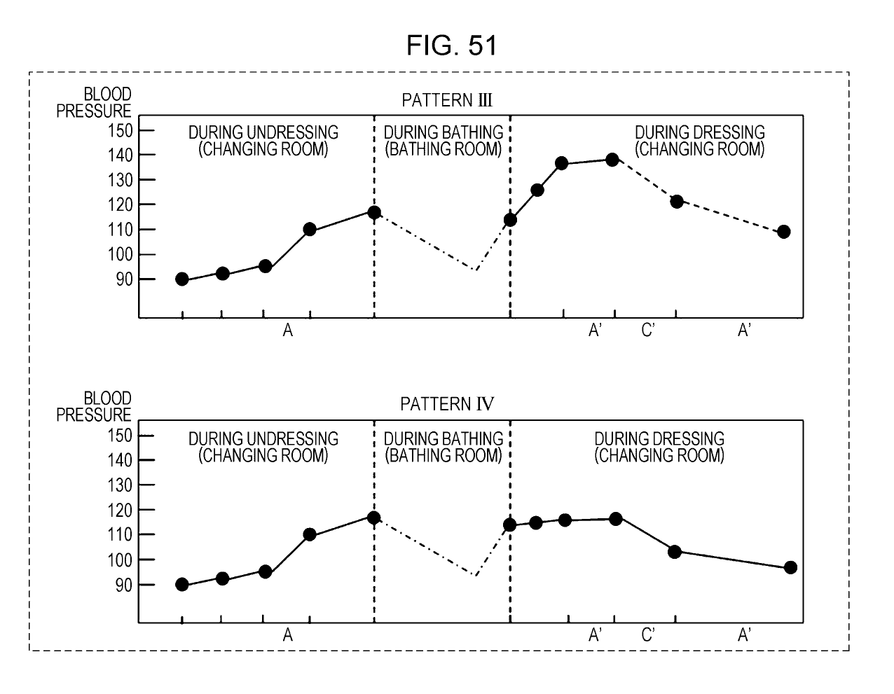

In pattern III illustrated in FIG. 51, the blood pressure increases while the user undresses, and the user with a blood pressure immediately before bathing of, for example, 115 mmHg has the same blood pressure of 115 mmHg upon entry into the changing room after bathing. Thereafter, the blood pressure increases by 20 mmHg or more up to the first dressing event.

That is, if the blood pressure immediately after entry into the changing room is within a fifth threshold of, for example, ±5 mmHG relative to the blood pressure immediately before entry into the bathing room, and if the increase in blood pressure over a period from immediately after entry into the changing room to the first dressing event is equal or greater than a sixth threshold of, for example, 20 mmHg, then the information presenter 106d determines this pattern of user's blood pressure variation as pattern III.

At this time, considering that blood pressure drops during bathing as the body is warmed, it is assumed that during the period from when the user gets out of the bathtub to when the user enters the changing room, the user already feels the cold and experiences an increase in blood pressure. That is, even though a blood pressure increase of 20 mmHg or more is measured over a period from entry into the changing room to the first dressing event, it is possible that the total increase in blood pressure is even greater, and the blood pressure may have increased by as much as 30 mmHg or more.

Accordingly, as with pattern II, the information presenter 106d presents a message “Please dress quickly.” on the display seen by the user, as the information to be presented as an alert for pattern III. For the third person, information indicating that the user is in a potentially dangerous condition may be presented to the mobile terminal of the third person.

In pattern IV illustrated in FIG. 51, as with pattern III, the blood pressure immediately before entry into the bathing room and the blood pressure upon entry into the changing room after bathing are substantially the same, with the blood pressure then remaining substantially unchanged up to the first dressing event.

That is, if the blood pressure immediately after entry into the changing room is within the fifth threshold of, for example, ±5 mmHG relative to the blood pressure immediately before entry into the bathing room, and if the increase in blood pressure over a period from immediately after bathing to the first dressing event is equal or greater than the sixth threshold of 20 mmHg, then the information presenter 106d determines this pattern of user's blood pressure variation as pattern IV.

At this time, as with pattern III, it is assumed that during the period from when the user gets out of the bathtub to when the user enters the changing room, the user already feels the cold and experiences an increase in blood pressure. However, since the blood pressure remains substantially constant after the re-entry into the changing room, it is determined that there is no abnormal blood pressure variation.

Accordingly, as the information to be presented as an alert for pattern IV, as with pattern I, the information presenter 106d notifies the user that the current situation is safe by constantly providing an indication to that effect on the display.

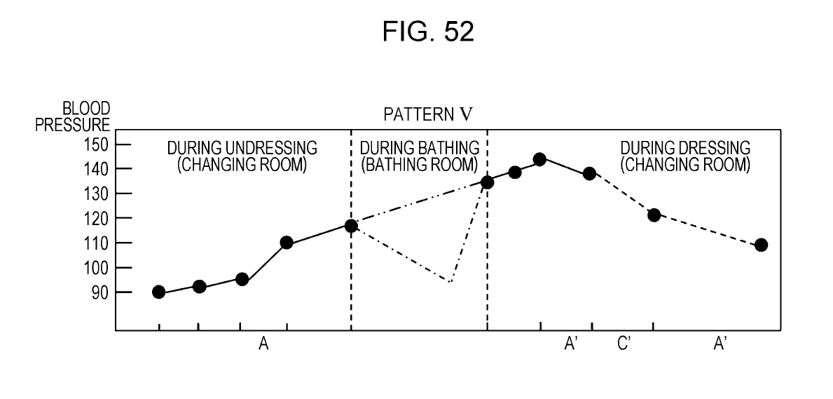

In pattern V illustrated in FIG. 52, the blood pressure immediately after entry into the changing room is higher than the blood pressure immediately before entry into the bathing room.

That is, if the blood pressure immediately after entry into the changing room is higher than the blood pressure immediately before entry into the bathing room, and the difference is greater than the fifth threshold, the information presenter 106d determines this pattern of user's blood pressure variation as pattern V.

At this time, there are two possible situations that may have occurred during bathing. The first is that the user's blood pressure continues to rise even during bathing, which indicates a potentially dangerous condition considering the increase in blood pressure during undressing. The second is that, as with pattern III or IV, the blood pressure drops once during bathing, but increases rapidly from when the user finishes bathing to when the user enters the changing room, which also indicates a condition that is potentially very dangerous. Accordingly, as with patterns II and III, a notification indicating that the current condition is dangerous is provided to the user or a third person as the information to be presented as an alert for pattern V. For pattern V, the user's condition can be determined to be dangerous immediately after the user enters the changing room. Accordingly, the alert information may be presented to the user immediately after entry into the changing room.

In the above-mentioned example, for patterns I and IV, the information presenter 106d provides a notification indicating that the current situation is safe on the display. However, this is not to be construed in a limiting sense. If the current situation is safe, there is no need to make the user aware of the alert information presenting apparatus, and thus no particular instruction may need to be given.

In the above-mentioned example, for patterns II, III, and V, the information presenter 106d presents information to the user by presenting details of an alert on the display. However, this is not to be construed in a limiting sense. For example, a heater may be activated to raise the temperature of the changing room. Further, the user may be alerted by sound.

Although the fourth threshold, the fifth threshold, and the sixth threshold used by the information presenter 106d are respectively 30 mmHg, ±5 mmHg, and 20 mmHg in the above-mentioned example, this is not to be construed in a limiting sense. For example, such thresholds may be set for each individual user, since the specific thresholds that allow determination of potentially dangerous conditions differ for each user.

In Embodiment 3, the blood pressure at the completion of undressing prior to bathing, and the blood pressures observed over a period from immediately after the user enters the changing room after bathing to the first dressing event are compared to determine the information to be presented as an alert, and the user is alerted accordingly by the information presenter 106d. However, this is not to be construed in a limiting sense. For example, since the user may possibly feel the cold even after the first dressing event, in an alternative configuration, blood pressure is measured up to a second dressing event following the first dressing event, and for pattern I and pattern II, the information to be presented as an alert may be determined based on whether the change in blood pressure exceeds the fourth threshold, and for pattern III and pattern IV, the determination may be made based on whether the change in blood pressure exceeds the sixth threshold.

In Embodiment 3, the information to be presented as an alert is determined by observing changes in blood pressure over a period from the entry into the changing room following bathing to the completion of the first dressing event. However, this is not to be construed in a limiting sense. If a change in blood pressure equal to or greater than the fourth threshold occurs before completion of the first dressing event, for example, at some point during the course of pattern II, an indication that the current situation is potentially dangerous may be presented to the user or a third person even before the completion of the first dressing event.

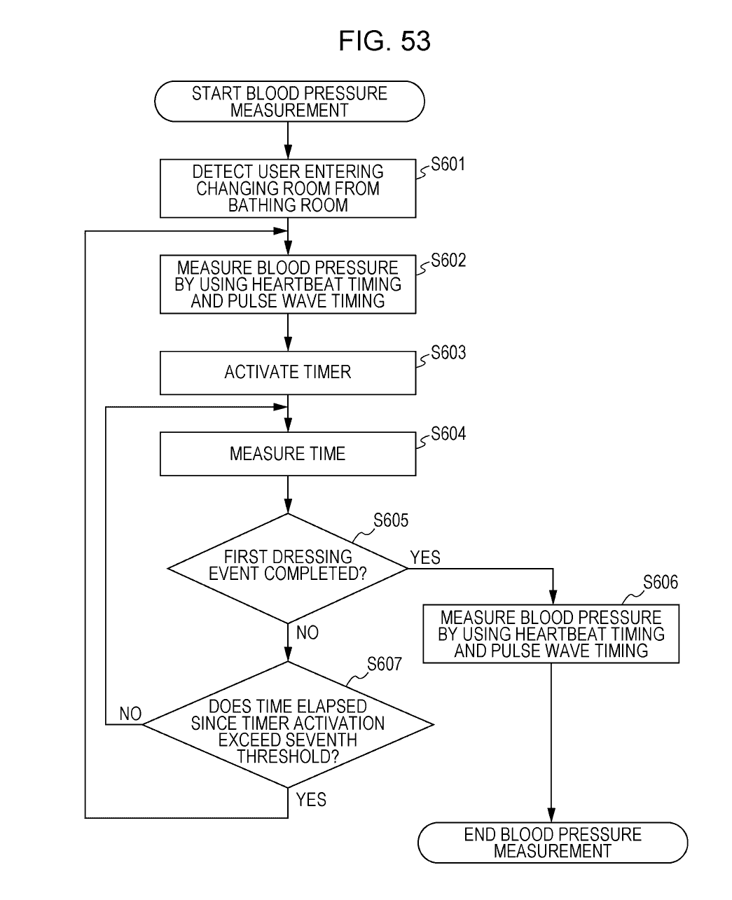

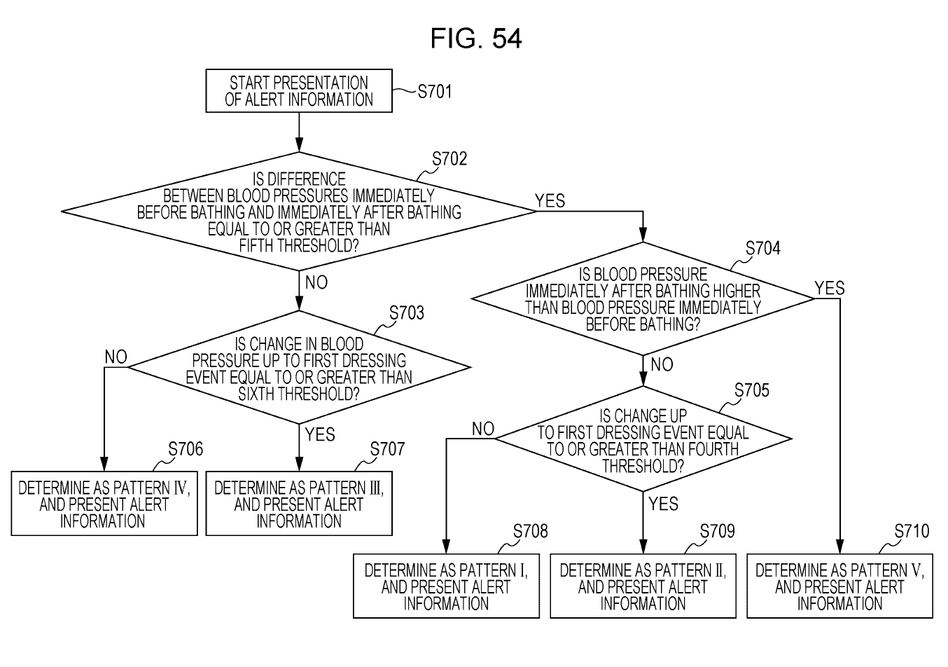

FIGS. 53 to 54 are flowcharts each illustrating operation of the alert information presenting apparatus 110d according to Embodiment 3. The alert information presenting apparatus 110d according to Embodiment 3 differs from Embodiment 1 in that alert information is presented to the user or a third person based on changes in blood pressure observed up to when the user dresses.

FIG. 53 is a flowchart illustrating determination of whether to measure blood pressure according to Embodiment 3.

First, the instant when the user enters the changing room from the bathing room is detected by using the signal acquirer 103d (step S601), and the pulse wave timing and the heartbeat timing at that time are respectively acquired by the pulse wave acquirer 102 and the heartbeat acquirer 104. Then, the blood pressure at the instant of entry into the changing room is calculated by the blood pressure calculator 105 (step S602). Next, the timer is activated (step S603), and the time elapsed since the entry into the changing room is measured (step S604). Then, the dressing condition determiner 109 determines the user has completed the first dressing event (step S605), and if the user has completed the first dressing event, blood pressure is calculated again by using pulse wave timing and heartbeat timing (step S606), and the blood pressure measurement is finished. If the user has not completed the first dressing event yet, it is checked whether the measured time since the activation of the timer exceeds a seventh threshold of, for example, one minute (step S607). If the seventh threshold has not been exceeded, the time measurement is continued, and it is determined again whether the user has completed the first dressing event. If the seventh threshold has been already exceeded, blood pressure is measured from the pulse wave timing and the heartbeat timing at that instant, and the same process is repeated until the first dressing event is completed.

FIG. 54 is a flowchart illustrating how the information to be provided as an alert is determined according to Embodiment 3.

After completion of the first dressing event, first, the blood pressure immediately before bathing and the blood pressure immediately after bathing are compared. Then, it is determined whether the comparison result indicates a difference equal to or greater than the fifth threshold (step S701). If the difference is equal to or greater than the fifth threshold, it is determined that the blood pressure immediately after bathing is higher than the blood pressure immediately before bathing (step S704). If the difference is less than the fifth threshold, it is determined whether the amount of change in blood pressure during the period from entry into the changing room to the first dressing event is equal to or greater than the sixth threshold (step S703). If it is determined in S704 that the blood pressure immediately after bathing is lower than the blood pressure immediately before bathing, it is determined whether the change in blood pressure during the period from entry into the changing room to the first dressing event is equal to or greater than the fourth threshold (step S705). If the change in blood pressure is less than the fourth threshold, the pattern of user's blood pressure variation is determined as pattern I (step S708). If the change in blood pressure is equal to or greater than the fourth threshold, the pattern of blood pressure variation is determined as pattern II, and alert information appropriate to the pattern is presented (step S709).

If it is determined in S703 that the change in blood pressure up to the first dressing event is less than the sixth threshold, the pattern of blood pressure variation is determined as pattern IV (step S706), and if the change in blood pressure is equal to or greater than the sixth threshold, the pattern of blood pressure variation is determined as pattern III, and alert information appropriate to the pattern is presented.

In the foregoing description of the present disclosure, when it is not possible to acquire the face image of the user for reasons such as the user's posture, an image of the user's hand is captured to acquire pulse wave timing. However, this is not to be construed in a limiting sense. The present disclosure particularly focuses on changes in blood pressure before and after movement A. The number of times movement A is performed differs depending on the season. For example, in winter time, movement A is performed at least twice to take off clothing items such as a sweater and an undershirt. Accordingly, if the user's posture at the time when movement A is performed for the first time makes it difficult to acquire pulse wave, a feedback to that effect may be given to the user to prompt the user to assume a posture that allows for easy acquisition of pulse wave.

Although one or more implementations of an alert information presenting apparatus and an alert information presenting method have been described above by way of various embodiments, the present disclosure is not limited to these embodiments. Implementations with various modifications as may occur to those skilled in the art made to the above embodiments, and implementations that combine constituent elements from different embodiments may also fall within the scope of the present disclosure without departing from the spirit of the present disclosure.

In the present disclosure, some or all of units and devices, or some or all of the functional blocks in the block diagrams illustrated in FIGS. 1 and 34 may be implemented by using one or more electronic circuits including a semiconductor device, a semiconductor integrated circuit (IC), or a large scale integration (LSI). An LSI or an IC may be integrated in one chip or may be made up of a combination of a plurality of chips. For example, functional blocks other than storage elements may be integrated in one chip. Although referred to as an LSI or IC here, the integrated circuit may be referred to as a system LSI, a very large scale integration (VLSI), or an ultra large scale integration (ULSI) depending on the degree of integration. A field programmable gate array (FPGA) that is programmed after manufacture of an LSI, or a reconfigurable logic device that allows reconfiguration of connections inside an LSI or allows set-up of circuit segments inside an LSI may be used for the same purpose.

Further, it is possible for some or all of the functions or operations of units, devices, or part of devices to be executed by software processing. In this case, software is recorded in one or more non-transitory recording media such as ROMs, optical discs, or hard disk drives, and when the software is executed by a processor, the software causes specific functions within the software to be executed by the processor and its peripheral devices. A system or device may include one or more non-transitory recording media on which the software is recorded, the processor, and a required hardware device, such as an interface. The software for implementing, for example, the alert information presenting apparatus according to each of the embodiments mentioned above causes a computer to execute the steps included in the flowcharts illustrated in FIGS. 26 to 30 and FIG. 38.

The present disclosure can be used for, for example, an alert information presenting apparatus that presents alert information based on blood pressure variation caused by undressing movement that takes place before bathing or before immersion in water.

What is claimed is:

1. An alert information presenting apparatus comprising:
a signal acquirer that acquires a receive signal including a radio wave reflected by a user;
an undressing condition determiner that determines, by using a movement speed of the user determined by using the receive signal and indicating a speed of a movement performed by the user,
a first time period indicating a period of time during which the user is undressing,
a second time period immediately preceding the first time period and indicating a period of time before the user undresses, and
a third time period immediately following the first time period and indicating a period of time after the user undresses;
a heartbeat acquirer that acquires, by using the receive signal, a pre-undressing heartbeat timing and a post-undressing heartbeat timing, the pre-undressing heartbeat timing indicating a heartbeat timing in the second time period, the post-undressing heartbeat timing indicating a heartbeat timing in the third time period;
a pulse wave acquirer that acquires a pre-undressing pulse wave timing and a post-undressing pulse wave timing, the pre-undressing pulse wave timing indicating a pulse wave timing in the second time period, the post-undressing pulse wave timing indicating a pulse wave timing in the third time period;
a blood pressure calculator that calculates a pre-undressing blood pressure of the user and a post-undressing blood pressure of the user, the pre-undressing blood pressure being calculated by using a time difference between the pre-undressing heartbeat timing and the pre-undressing pulse wave timing, the post-undressing blood pressure being calculated by using a time difference between the post-undressing heartbeat timing and the post-undressing pulse wave timing; and
an information presenter that presents the user with alert information based on a difference between the pre-undressing blood pressure and the post-undressing blood pressure.

2. The alert information presenting apparatus according to claim 1,
wherein the undressing condition determiner determines, as the first time period, a period of time during which the receive signal shows a movement speed equal to or greater than a first threshold,
wherein the undressing condition determiner determines, as the second time period, a period of time immediately preceding the first time period and during which the receive signal shows a movement speed less than the first threshold, and
wherein the undressing condition determiner determines, as the third time period, a period of time immediately following the first time period and during which the receive signal shows a movement speed less than the first threshold.

3. The alert information presenting apparatus according to claim 1,
wherein the heartbeat acquirer acquires, as the pre-undressing heartbeat timing, a time point corresponding to a peak of a time waveform in the second time period of the receive signal, and
wherein the heartbeat acquirer acquires, as the post-undressing heartbeat timing, a time point corresponding to a peak of a time waveform in the third time period of the receive signal.

4. The alert information presenting apparatus according to claim 1, further comprising
an image acquirer that acquires an image including a skin of the user, wherein the pulse wave acquirer acquires, as the pre-undressing pulse wave timing, a time point corresponding to a peak of a time waveform in the second time period of a signal that indicates time variation of a luminance of the skin of the user in the image, and
wherein the pulse wave acquirer acquires, as the post-undressing pulse wave timing, a time point corresponding to a peak of a time waveform in the third time period of the signal that indicates the time variation of the luminance of the skin of the user in the image.

5. The alert information presenting apparatus according to claim 1,
wherein the information presenter presents, if the difference between the pre-undressing blood pressure and the post-undressing blood pressure is equal to or greater than a second threshold, alert information that prompts the user to refrain from immersion in water.

6. The alert information presenting apparatus according to claim 1,
wherein the undressing condition determiner determines, as the first time period, a period of time in the receive signal during which an area corresponding to a right shoulder of the user and an area corresponding to a left shoulder of the user simultaneously show movement speeds equal to or greater than the first threshold.

7. The alert information presenting apparatus according to claim 1,
wherein the undressing condition determiner determines, as the first time period, a period of time in the receive signal during which an area corresponding to a central part of an upper body of the user shows a movement speed equal to or higher than the first threshold.

8. The alert information presenting apparatus according to claim 1, further comprising
a pulse wave sensor that detects a pulse wave of the user,
wherein the pulse wave acquirer acquires the pre-undressing pulse wave timing for the second time period and the post-undressing pulse wave timing for the third time period by using a signal indicative of the pulse wave detected by the pulse wave sensor.

9. The alert information presenting apparatus according to claim 6,
wherein the undressing condition determiner further includes an undressing-progression determiner to recognize that the user has stopped undressing when the area corresponding to the right shoulder of the user and the area corresponding to the left shoulder of the user simultaneously show movement speeds equal to or greater than the first threshold and then the movement speeds become zero before movement is made past areas corresponding to both the right shoulder and the left shoulder of the user.

10. The alert information presenting apparatus according to claim 1, further comprising
an eyeglass donning and doffing determiner,
wherein whether the user is wearing eyeglasses is determined by using the image acquirer, and when the user removes the eyeglasses, the signal acquirer and the pulse wave acquirer are activated, and an undressing condition is determined by using the undressing condition determiner.

11. The alert information presenting apparatus according to claim 1, further comprising
a dressing condition determiner,
wherein a dressing condition of the user after bathing is determined, blood pressures before and after the user dresses are measured by using the blood pressure calculator, entry of the user into a changing room after bathing is recognized by using the signal acquirer, and a blood pressure at a first time representing a time of the entry is measured by using the blood pressure calculator.

12. An alert information presenting method comprising:
(a) acquiring a receive signal including a radio wave reflected by a user;
(b) determining, by using a movement speed of the user determined by using the receive signal and indicating a speed of a movement performed by the user,
a first time period indicating a period of time during which the user is undressing,
a second time period immediately preceding the first time period and indicating a period of time before the user undresses, and
a third time period immediately following the first time period and indicating a period of time after the user undresses;
(c) acquiring, by using the receive signal, a pre-undressing heartbeat timing and a post-undressing heartbeat timing, the pre-undressing heartbeat timing indicating a heartbeat timing in the second time period, the post-undressing heartbeat timing indicating a heartbeat timing in the third time period;
(d) acquiring a pre-undressing pulse wave timing and a post-undressing pulse wave timing, the pre-undressing pulse wave timing indicating a pulse wave timing in the second time period, the post-undressing pulse wave timing indicating a pulse wave timing in the third time period;
(e) calculating a pre-undressing blood pressure of the user and a post-undressing blood pressure of the user, the pre-undressing blood pressure being calculated by using a time difference between the pre-undressing heartbeat timing and the pre-undressing pulse wave timing, the post-undressing blood pressure being calculated by using a time difference between the post-undressing heartbeat timing and the post-undressing pulse wave timing; and
(f) presenting the user with alert information based on a difference between the pre-undressing blood pressure and the post-undressing blood pressure.

13. The alert information presenting method according to claim 12,
wherein the step (b) comprises
determining, as the first time period, a period of time during which the receive signal shows a movement speed equal to or greater than a first threshold,
determining, as the second time period, a period of time immediately preceding the first time period and during which the receive signal shows a movement speed less than the first threshold, and
determining, as the third time period, a period of time immediately following the first time period and during which the receive signal shows a movement speed less than the first threshold.

14. The alert information presenting method according to claim 12,
wherein the step (c) comprises
acquiring, as the pre-undressing heartbeat timing, a time point corresponding to a peak of a time waveform in the second time period of the receive signal, and
acquiring, as the post-undressing heartbeat timing, a time point corresponding to a peak of a time waveform in the third time period of the receive signal.

15. The alert information presenting method according to claim 12, further comprising
acquiring an image including a skin of the user,
wherein the step (d) comprises
acquiring, as the pre-undressing pulse wave timing, a time point corresponding to a peak of a time waveform in the second time period of a signal that indicates time variation of a luminance of the skin of the user in the image, and
acquiring, as the post-undressing pulse wave timing, a time point corresponding to a peak of a time waveform in the third time period of the signal that indicates the time variation of the luminance of the skin of the user in the image.

16. The alert information presenting method according to claim 12,
wherein the step (f) comprises presenting, if the difference between the pre-undressing blood pressure and the post-undressing blood pressure is equal to or greater than a second threshold, alert information that prompts the user to refrain from immersion in water.

17. The alert information presenting method according to claim 12,
wherein the step (b) comprises determining, as the first time period, a period of time in the receive signal during which an area corresponding to a right shoulder of the user and an area corresponding to a left shoulder of the user simultaneously show movement speeds equal to or greater than the first threshold.

18. The alert information presenting method according to claim 12,
wherein the step (b) comprises determining, as the first time period, a period of time in the receive signal during which an area corresponding to a central part of an upper body of the user shows a movement speed equal to or higher than the first threshold.

19. The alert information presenting method according to claim 12, further comprising
detecting a pulse wave of the user by using a pulse wave sensor,
wherein the step (d) comprises acquiring the pre-undressing pulse wave timing for the second time period and the post-undressing pulse wave timing for the third time period by using a signal indicative of the pulse wave detected by the pulse wave sensor.

20. A blood pressure information outputting method comprising,
receiving, on detectors, radio waves reflected by a user during a time period;
acquiring pairs of information items during the time period, each of the pairs of information items including a luminance of a skin of the user and a time at which the luminance is acquired;
determining information, based on the received reflected radio waves, including movement speeds of portions of the user;
determining a first time period within the time period, the first time period including a fourth time period and a fifth time period, the fourth time period being a period of time during which each of movement speeds of first portions included in the portions is greater than a first speed, the fifth time period being a period of time during which each of movement speeds of second portions included in the portions is greater than a second speed, the first speed being greater than the second speed;
determining a second time period within the time period, the second time period immediately preceding the first time period, the second time period being a period of time during which each of the movement speeds of the portions is less than the second speed;
determining a third time period within the time period, the third time period immediately following the first time period, the third time period being a period of time during which each of the movement speeds of the portions is less than the second speed; and
outputting information indicating a change in blood pressure of the user, the information being based on a part of the reflected radio waves and a part of the pairs of information items, a time period of the part of the reflected radio waves being the second time period and the third time period, a time period of the part of the pairs of information items being the second time period and the third time.
